(19) United States
(12) Patent Application Publication
(10) Pub. No.: US 2021/0139889 A1
(43) Pub. Date: May 13, 2021

(54) Compositions and Methods for Multiplexed Genome Editing and Screening

(71) Applicant: YALE UNIVERSITY, New Haven, CT (US)

(72) Inventors: Sidi Chen, Milford, CT (US); Ryan Chow, San Jose, CA (US)

(21) Appl. No.: 16/624,775

(22) PCT Filed: Jun. 19, 2018
(86) PCT No.: PCT/US18/38242

§ 371 Date: Dec. 19, 2019

Related U.S. Application Data
(60) Provisional application No. 62/521,600, filed on Jun. 19, 2017.
(60) Provisional application No. 62/660,467, filed on Apr. 20, 2018.

(51) Int. Cl. C12N 15/10; C12N 15/85; C12N 9/22

(52) U.S. Cl. CPC C12N 15/1082; C12N 15/1024; C12N 15/85; C12N 2310/20; C12N 2800/80; C12N 2830/00; C12N 9/22

(57) ABSTRACT

The present invention includes compositions and methods for multiplexed genome editing and screening in vivo. In certain aspects, the invention includes an CCAS library for multiplexed genome-scale mutagenesis.

### CROSS-REFERENCE TO RELATED APPLICATION

The present application is entitled to priority under 35 U.S.C. § 119(e) to U.S. Provisional Patent Application No. 62/521,600, filed Jun. 19, 2017, and U.S. Provisional Patent Application No. 62/660,467, filed Apr. 20, 2018, which are both incorporated by reference in their entireties herein.

### STATEMENT REGARDING FEDERALLY SPONSORED RESEARCH OR DEVELOPMENT

This invention was made with government support under CA21974, CA209992, CA196530, and GM007205 awarded by National Institutes of Health. The government has certain rights in the invention.

### BACKGROUND OF THE INVENTION

Genetic interactions lay the foundation of virtually all biological systems. With rare exceptions, every gene interacts with one or more other genes, forming highly complex and dynamic networks. The nature of genetic interactions includes physical interactions, functional redundancy, enhancer, suppressor, and/or synthetic lethality. Such interactions are the cornerstones of biological processes such as embryonic development, homeostatic regulation, immune responses, nervous system function and behavior, and evolution. Perturbation or misregulation of genetic interactions in the germ line can lead to failures in development, physiological malfunction, autoimmunity, neurological disorders, and/or many forms of genetic diseases. Disruption of the genetic networks in somatic cells can lead to malignant cellular behaviors such as uncontrolled growth, driving the development of cancer.

The study of genetic interactions evolved over a century, originating in the era of classical genetics. In essence, how two genes interact can be studied by examining the phenotypes of double mutants as compared to single mutants. This concept of epistasis has guided the conceptualization and subsequent discovery of countless important pathways, and has become the gold standard for determining downstream and upstream regulation in genetic analysis. For instance, synthetic lethality has been investigated in animal development and cancer therapeutics. Classical approaches such as genome-wide association studies (GWAS) and quantitative trait loci (QTL) mapping have been extensively employed to study complex phenotypes that involve multiple genes. While high-throughput genetic perturbation approaches have been developed to map out the landscape of genetic interactions in yeast and in worms, large-scale double knockout studies in mammalian species are scarce, due to the exponentially scaling number of possible gene combinations and the technological challenges of generating and screening double knockouts.

There is thus a need in the art for compositions and methods for high-throughput multi-dimensional knockout screening. Such compositions and methods should be useful for multiplexed genome editing and screening. The present invention satisfies this need.

### SUMMARY OF THE INVENTION

As described herein, the present invention relates to compositions and methods for simultaneously or sequentially mutagenizing multiple target sequences in a cell.

One aspect of the invention includes a vector comprising a first long terminal repeat (LTR) sequence, an Embryonal Fyn-Associated Substrate (EFS) sequence, a Cpf1 sequence, a Nuclear Localization Signal (NLS) sequence, an antibiotic resistance sequence, and a second LTR sequence.

Another aspect of the invention includes a vector comprising a first LTR sequence, a promoter sequence, a direct repeat sequence of Cpf1, a first restriction site, a second restriction site, an EFS sequence, an antibiotic resistance sequence, a posttranscriptional regulatory element sequence, and a second LTR sequence.

Yet another aspect of the invention includes a crRNA array comprising a 5′ nucleotide sequence that is homologous to a first nucleotide sequence on a vector, a first crRNA sequence, a direct repeat sequence of Cpf1, a second crRNA sequence, a terminator sequence, and a 3′ sequence that is homologous to a second sequence on the vector.

In another aspect, the invention includes a vector comprising a first LTR sequence, a promoter sequence, a first direct repeat sequence of Cpf1, a first crRNA sequence, a second direct repeat sequence of Cpf1, a second crRNA sequence, a terminator sequence, an EFS sequence, a posttranscriptional regulatory sequence, and a second LTR sequence.

In yet another aspect, the invention includes a crRNA library comprising a plurality of crRNA arrays cloned into a plurality of vectors, wherein the crRNA arrays individually comprise a 5′ nucleotide sequence that is homologous to a first nucleotide sequence on a vector, a first crRNA sequence, a direct repeat sequence of Cpf1, a second crRNA sequence, a terminator sequence, and a 3′ sequence that is homologous to a second sequence on the vector.

In still another aspect, the invention includes a method for simultaneously mutagenizing multiple target sequences in a cell. The method comprises administering to the cell a crRNA library comprising a plurality of vectors comprising a plurality of crRNA arrays. Each crRNA array independently comprises a 5′ nucleotide sequence that is homologous to a first nucleotide sequence on the vector, a first crRNA sequence, a direct repeat sequence of Cpf1, a second crRNA sequence, a terminator sequence, and a 3′ sequence that is homologous to a second sequence on the vector. The first crRNA is complementary to a first target sequence and the second crRNA is complementary to a second target sequence.

Another aspect of the invention includes a method of identifying synergistic drivers of transformation and/or tumorigenesis in vivo. The method comprises administering a cell mutagenized by a crRNA library to an animal. The crRNA library comprises a plurality of vectors comprising a plurality of crRNA arrays. Each crRNA array independently comprises a 5′ nucleotide sequence that is homologous to a first nucleotide sequence on the vector, a first crRNA sequence, a direct repeat sequence of Cpf1, a second crRNA sequence, a terminator sequence and a 3′ sequence that is homologous to a second sequence on the vector. The first crRNA is complementary to a first target sequence and the second crRNA is complementary to a second target sequence. A nucleotide from a tumor from the animal is sequenced. The data from the sequencing are analyzed to identify the synergistic drivers of transformation and/or tumorigenesis.

Yet another aspect of the invention includes an in vivo method for identifying and mapping genetic interactions between a plurality of genes. The method comprises administering a cell mutagenized by a crRNA library to an animal. The crRNA library comprises a plurality of vectors comprising a plurality of crRNA arrays. The crRNA array comprises a 5′ nucleotide sequence that is homologous to a first nucleotide sequence on the vector, a first crRNA sequence, a direct repeat sequence of Cpf1, a second crRNA sequence, a terminator sequence, and a 3′ sequence that is homologous to a second sequence on the vector. The first crRNA is complementary to a first target sequence and the second crRNA is complementary to a second target sequence. A nucleotide from a tissue from the animal is sequence. The data from the sequencing are analyzed to identify and map the genetic interactions.

Another aspect of the invention includes a kit comprising a CCAS library comprising a plurality of vectors comprising a plurality of crRNA arrays, wherein the crRNA arrays comprise a nucleotide sequence selected from the group consisting of SEQ ID NOs: 4-9,708, and instructional material for use thereof.

Still another aspect of the invention includes a kit comprising a MCAP library comprising a plurality of vectors comprising a plurality of crRNA arrays, wherein the crRNA arrays comprise a nucleotide sequence selected from the group consisting of SEQ ID NOs: 9,762-21,695, and instructional material for use thereof.

In another aspect, the invention includes a vector comprising a first promoter, a Cpf1 sequence, a second promoter, a first Cpf1 direct repeat sequence, a lox66 sequence, a second Cpf1 direct repeat sequence, two inverted restriction sites, an inverted lox71 sequence, and a crRNA FlipArray. The crRNA FlipArray comprises a first crRNA sequence, 4-10 consecutive thymidines, a second inverted crRNA sequence, 4-10 consecutive adenines, and a third inverted direct repeat sequence.

In yet another aspect, the invention includes a gene editing system capable of inducible, sequential mutagenesis in a cell. The system comprises a vector and a Cre recombinase. The vector comprises a first promoter, a Cpf1 sequence, a second promoter, a first Cpf1 direct repeat sequence, a lox66 sequence, a second Cpf1 direct repeat sequence, two inverted restriction sites, an inverted lox71 sequence, and a crRNA FlipArray. The crRNA FlipArray comprises a first crRNA sequence, 4-10 consecutive thymidines, a second inverted crRNA sequence, 4-10 consecutive adenines, and a third inverted direct repeat sequence.

Another aspect of the invention includes a gene editing system capable of inducible, sequential mutagenesis in a cell. The system comprises a plurality of vectors and a Cre recombinase. The the vectors comprise a first promoter, a Cpf1 sequence, a second promoter, a first Cpf1 direct repeat sequence, a lox66 sequence, a second Cpf1 direct repeat sequence, two inverted restriction sites, an inverted lox71 sequence, and a crRNA FlipArray. The crRNA FlipArray comprises a first crRNA sequence, 4-10 consecutive thymidines, a second inverted crRNA sequence, 4-10 consecutive adenines, and a third inverted direct repeat sequence.

Yet another aspect of the invention includes a method of inducible, sequential mutagenesis in a cell. The method comprises administering to the cell a vector comprising a first promoter, a Cpf1 sequence, a second promoter, a first Cpf1 direct repeat sequence, a lox66 sequence, a second Cpf1 direct repeat sequence, two inverted restriction sites, an inverted lox71 sequence, and a crRNA FlipArray. The crRNA FlipArray comprises a first crRNA sequence, 4-10 consecutive thymidines, a second inverted crRNA sequence, 4-10 consecutive adenines, and a third inverted direct repeat sequence. The first crRNA is expressed. A Cre recombinase is administered to the cell. When the Cre recombinase is administered, the second crRNA is expressed, thus sequentially mutagenizing the cell.

Still another aspect of the invention includes a method of inducible, sequential mutagenesis in a cell. The method comprises administering to the cell a plurality of vectors. The plurality of vectors individually comprise a first promoter, a Cpf1 sequence, a second promoter, a first Cpf1 direct repeat sequence, a lox66 sequence, a second Cpf1 direct repeat sequence, two inverted restriction sites, an inverted lox71 sequence, and a crRNA FlipArray. The crRNA FlipArray comprises a first crRNA sequence, 4-10 consecutive thymidines, a second inverted crRNA sequence, 4-10 consecutive adenines, and a third inverted direct repeat sequence. The first crRNA is expressed. A Cre recombinase is administered to the cell. When the Cre recombinase is administered, the second crRNA is expressed, thus sequentially mutagenizing the cell.

Another aspect of the invention includes a method of inducible, sequential mutagenesis in a cell in an animal. The method comprises administering to the animal a plurality of vectors. The plurality of vectors individually comprise a first promoter, a Cpf1 sequence, a second promoter, a first Cpf1 direct repeat sequence, a lox66 sequence, a second Cpf1 direct repeat sequence, two inverted restriction sites, an inverted lox71 sequence, and a crRNA FlipArray. The crRNA FlipArray comprises a first crRNA sequence, 4-10 consecutive thymidines, a second inverted crRNA sequence, 4-10 consecutive adenines, and a third inverted direct repeat sequence. The first crRNA is expressed. The animal is administered a Cre recombinase. When the Cre recombinase is administered, the second crRNA is expressed thus sequentially mutagenizing the cell in the animal.

In various embodiments of the above aspects or any other aspect of the invention delineated herein, the vector further comprises a tag sequence. In one embodiment, the tag sequence is a a Flag2A sequence. In one embodiment, the first and/or second restriction site is a BsmBI restriction site. In one embodiment, the posttranscriptional regulatory element sequence comprises a Woodchuck Hepatitis Virus (WHP) Posttranscriptional Regulatory Element (WPRE) sequence. In one embodiment, the promoter sequence comprises a U6 promoter sequence. In one embodiment, the terminator sequence comprises a U6 terminator sequence.

In one embodiment, the first promoter is an EFS promoter. In one embodiment, the EFS promoter drives expression of Cpf1. In one embodiment, the second promoter is a U6 promoter. In one embodiment, the U6 promoter drives expression of the crRNA FlipArray. In one embodiment, the first promoter and the second promoter are in opposite orientations. In one embodiment, the vector further comprises an antibiotic resistance marker. In one embodiment, In one embodiment, the antibiotic resistance marker is a puromycin resistance sequence. In one embodiment, the restriction sites are BsmbI restriction sites. In one embodiment, the Cpf1 sequence is a Lachnospiraceae bacterium Cpf1 (LbCpf1) sequence. In one embodiment, any one of the first, second, or third, direct repeat sequences is from LbCpf1. In one embodiment, the first crRNA sequence comprises six consecutive thymidines. In one embodiment, the second inverted crRNA sequence comprises six consecutive adenines. In one embodiment, the first crRNA and/or the second crRNA target more than one sequence.

In one embodiment, the vector comprises the nucleic acid sequence of SEQ ID NO: 1. In one embodiment, the vector comprises the nucleic acid sequence of SEQ ID NO: 2. In one embodiment, the vector comprises SEQ ID NO: 21,697.

In one embodiment, the crRNA array comprises any one of the vectors of the present invention. In one embodiment, the crRNA library comprises any one of the vectors of the present invention.

In one embodiment, the first crRNA sequence is complementary to a gene selected from the group consisting of Pten and Nf1, and the second crRNA sequence is complementary to a gene selected from the group consisting of Pten and Nf1. In one embodiment, the first crRNA targets Nf1 and the second crRNA targets Pten. In one embodiment, the first crRNA and/or the second crRNA targets a panel of immunomodulatory factors comprising Cd274, Ido1, B2m, Fas1, Jak2, and Lgals9.

In one embodiment, the crRNA arrays comprise a nucleotide sequence selected from the group consisting of SEQ ID NOs: 4-9,708. In one embodiment, the crRNA arrays comprise a nucleotide sequence selected from the group consisting of SEQ ID NOs: 9,762-21,695. In one embodiment, the plurality of crRNA arrays comprise a nucleotide sequence selected from the group consisting of SEQ ID NOs. 4-9,708. In one embodiment, the plurality of crRNA arrays comprise a nucleotide sequence selected from the group consisting of SEQ ID NOs. 9,762-21,695. In one embodiment, the crRNA comprises at least one additional crRNA sequence that is complementary to at least one additional target sequence. In one embodiment, the first crRNA and/or the second crRNA targets more than one sequence.

In one embodiment, the crRNA library comprises a Cpf1 crRNA array screening (CCAS) library, wherein the crRNA arrays consist of SEQ ID NOs: 4-9,708. In one embodiment, the crRNA library comprises a Massively-Parallel crRNA Array Profiling (MCAP) library comprising a plurality of crRNA arrays targeting pairwise combinations of genes significantly mutated in human metastases. In one embodiment, the MCAP library comprises crRNA arrays consisting of SEQ ID NOs: 9,762-21,695.

In one embodiment, the cell is selected from the group consisting of a T cell, a CD8+ cell, a CD4+ cell, a dendritic cell, an endothelial cell, and a stem cell. In one embodiment, the cell is a human cell. In one embodiment, the animal is a mouse. In one embodiment, the animal is a human.

In one embodiment, the mutagenesis is selected from the group consisting of nucleotide insertion, nucleotide deletion, frameshift mutation, gene activation, gene repression, and epigenetic modification.

### BRIEF DESCRIPTION OF THE DRAWINGS

The following detailed description of specific embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIGS. 1A-1D are a series of plots and images illustrating enabling one-step double knockout screening with a Cpf1 crRNA array library. FIG. 1A shows schematic maps of the constructs for one-step double knockout screens by CRISPR-Cpf1. A pLenti-EFS-Cpf1-blast vector, which constitutively expresses a humanized form of Lachnospiraceae bacterium Cpf1 (LbCpf1) was generated; transduced cells can be selected by blasticidin. A pLenti-U6-DR-crRNA-puro vector, which contains the direct repeat (DR) sequence of Cpf1 and two BsmBI restriction sites for one-step cloning of crRNA arrays, was also generated; puromycin treatment enables the selection of cells that have been transduced. The structure of the crRNA array library for cloning into the base vector is also shown. Each crRNA array is comprised of a 5′ homology arm to the base vector, followed by the first crRNA, the direct repeat (DR) sequence for Cpf1, the second crRNA, a U6 terminator sequence, and a 3′ homology arm. FIG. 1B is a schematic of the cloning strategy for double knockout screens by CRISPR-Cpf1. Incorporating a crRNA array library into the base vector simply requires BsmBI linearization followed by Gibson assembly, thereby producing a lentiviral version of the library (pLenti-U6-DR-cr1(N20)-DR-cr2(N20)-puro). This one-step cloning procedure greatly simplifies library construction for high-dimension genetic screens. FIG. 1C is a schematic describing the design and synthesis of the Cpf1 double knockout (CCAS) library for identifying synergistic drivers of tumorigenesis. The top 50 tumor suppressors (TSGs) were first identified based on an unbiased pan-cancer analysis of 17 cancer types from the TCGA (PANCAN17-TSG50). 49 of these 50 TSGs had corresponding mouse orthologs (PANCAN17-mTSG). All possible Cpf1 spacer sequences within these genes were identified, and 2 were chosen for each gene. The selection of crRNAs was based on two scoring criteria: 1) high genome-wide mapping specificity and 2) a low number of consecutive thymidines, since long stretches of thymidines will terminate U6 transcription. With these 98 crRNAs and 3 additional non-targeting control (NTC) crRNAs, a library was designed containing 9,705 permutations of two crRNAs each (CCAS library). After pooled oligo synthesis, the PANCAN17-mTSG CCAS library was cloned into the base vector, and the plasmid crRNA array representation was subsequently read out by deep-sequencing the crRNA expression cassette. FIG. 1D is a density plot showing the distribution of CCAS crRNA array abundance in terms of log₂ reads per million (rpm). Of the 9,705 total crRNA arrays in the library, 9,408 were comprised of two gene-targeting crRNAs (double knockout, or DKO), while 294 contained one gene-targeting crRNA and one NTC crRNA (single knockout, or SKO). The remaining 3 crRNA arrays were controls, with two NTC crRNAs in the crRNA array (NTC-NTC, not shown). The library-wide abundance of both DKO and SKO crRNA arrays followed a log-normal distribution, demonstrating relatively even coverage of the CCAS plasmid library.

FIGS. 2A-2E are a series of plots and images illustrating a library-scale Cpf1 crRNA array screen in a mouse model of early tumorigenesis. FIG. 2A is a schematic of the experimental approach for Cpf1-mediated double knockout screens to identify synergistic drivers of tumorigenesis in a transplant model. Lentiviral pools were generated from the CCAS plasmid library, and subsequently infected Cpf1+IM cells to perform massively parallel gene-pair level mutagenesis. The mixed double mutant cell population (CCAS-treated cells), or vector-treated control cells were then injected subcutaneously into nude mice (n=10 and n=4, respectively). After 6.5 weeks, genomic DNA was extracted from the injection site and subjected to crRNA array sequencing. FIG. 2B shows tumor growth curves of CCAS-treated (red, n=10) and vector-treated cells (black, n=4) in vivo. As expected, vector treated cells were lowly tumorigenic, and the population of mixed double mutants (CCAS-treated cells) were highly tumorigenic. By 45 days post injection (dpi), tumors derived from CCAS-treated cells were significantly larger than those by vector-treated cells (* p<0.05, ** p<0.01, two-sided t-test). FIG. 2C shows histological sections of tumors derived from vector-treated and CCAS-treated cells, stained by hematoxylin and eosin. Two representative tumors are shown from each group. Images in each row were taken at the same magnification (top row, scale bar=500 μm; bottom row, scale bar=200 μm). CCAS-treated cells gave rise to much larger tumors than vector-treated cells. FIG. 2D is a dot-boxplot depicting the overall representation of the CCAS library, in terms of log₂ rpm abundance. The plasmid library, 4 pre-injection cell pools, and 10 tumor samples were sequenced. NTC-NTC controls, SKO crRNA arrays, and DKO crRNA arrays are shown. Whereas plasmid and cell samples exhibited lognormal representation of the CCAS library, tumor samples showed strong enrichment of specific SKO and DKO crRNA arrays. Notably, NTC-NTC crRNA arrays were consistently found at low abundance in all tumor samples. FIG. 2E is a scatterplot comparing average log 2 rpm abundance of all CCAS crRNA arrays in cells and in tumors. The linear regression line is shown, demonstrating the log-linear relationship of most crRNA arrays between tumors and cells (r²=0.166, coefficient=0.569, p<2.2 e-16 by F-test). There were numerous outliers (Bonferroni adjusted p<0.05), indicating that specific crRNA arrays had undergone positive selection in vivo. See FIGS. 10A-10B for the individual tumor comparisons, with outliers labeled.

FIGS. 3A-3E are a series of plots and images illustrating enrichment analysis of single knockout and double knockout crRNA arrays. FIG. 3A shows ranked crRNA array abundance plots of four representative tumor samples. In each tumor, there was a distinct set of DKO crRNA arrays that showed clear enrichment above the rest of the library, including the corresponding SKO crRNA arrays for each DKO pair. In Tumor 1, crCasp8.crApc was by far the most abundant crRNA array, dwarfing all other crRNA arrays including the corresponding SKO crRNA arrays crApc.NTC and crCasp8.NTC. Tumor 3 was dominated by crSetd2.crAcvr2a and crRnf43.crAtrx, Tumor 5 by crCic.crZc3h13 and crCbwd1.crNsd1, and Tumor 6 by crAtm.crRunx1 and crKmt2d.crH2-Q2. In all of these cases, the corresponding SKO crRNA arrays were far less abundant compared to the DKO crRNA arrays. FIG. 3B shows a volcano plot of DKO and SKO crRNA arrays compared to NTC-NTC controls. Log 2 fold change is calculated using average log 2 rpm abundance across all tumor samples, after averaging the 3 NTC-NTC controls to get one NTC-NTC score per sample. 655 crRNA arrays were found to be significantly enriched compared to NTC-NTC controls (Benjamini Hochberg-adjusted p<0.05). Of these, 620 were DKO crRNA arrays and 354 were SKO crRNA arrays. In total, the 655 enriched crRNA arrays corresponded to 498 gene combinations. A Venn diagram is also shown, detailing the number of genes involved in significant DKO and/or SKO crRNA arrays. All 49 genes in the PANCAN17-mTSG CCAS library were represented within at least one significant DKO crRNA array, while 24 genes were found to be significant as part of a SKO crRNA array. FIG. 3C is a bar plot of the top 10 genes ranked by the number of significant crRNA arrays associated with each gene. DKO crRNA array counts are shown in light grey, and SKO crRNA arrays in dark grey. Rnf43 and Kmt2c were the two most influential genes, associated with 58 and 51 independent crRNA arrays. FIG. 3D is a bar plot showing the number of significant DKO crRNA arrays associated with each gene pair in the CCAS library. 113 gene pairs were represented by at least 2 independent DKO crRNA arrays. Of note, the interaction of Atrx+Setd2 was supported by 5 independent crRNA arrays, while Atrx+Kmt2c, Arid1a+Map3k1, Kdm5c+Kmt2c, and Arid1a+Rnf43 were substantiated by 4 crRNA arrays. FIG. 3E is a violin plot showing the distribution of permutation correlations between crX.crY and crY.crX for the 4,704 DKO crRNA array combinations in the CCAS library (9,408 unique crRNA array permutations). In total, 80.1% (3,767/4,704) of all crRNA array combinations were significantly correlated when comparing the two permutations associated with each combination (Benjamini-Hochberg adjusted p<0.05, by t-distribution).

FIGS. 4A-4E are a series of plots and images illustrating high-throughput identification of synergistic gene pairs as co-drivers of transformation and tumorigenesis. FIG. 4A is a schematic describing the methodology for calculating a synergy coefficient (SynCo) for each DKO crRNA array in individual tumor samples. DKOₓ score is the log₂ rpm abundance of the DKO crRNA array (i.e., crX.crY) after subtracting average NTC-NTC abundance. SKOₓ and SKO_{y} scores are defined as the average log₂ rpm abundance of each SKO crRNA array (3 SKO crRNA arrays associated with each individual crRNA), after subtracting average NTC-NTC abundance. SynCo=DKO_{xy}−SKOₓ−SKO_{y}. By this definition, a SynCo score>>0 would indicate that a given DKO crRNA array is synergistic, as the DKO score would thus be greater than the sum of the individual SKO scores. FIG. 4B is a volcano plot of average SynCo across all tumor samples and associated −log₁₀ Benjamini-Hochberg adjusted p-value (two-sided one sample t-test, Ho: mean SynCo=0) for each DKO crRNA array in the library. Each point is scaled by size, in reference to the % of tumor samples with a SynCo≥7 for a particular crRNA array, and also color-coded according to the average log₂ rpm abundance across all tumor samples. To the right is a zoomed-in view of the top synergistic DKO crRNA arrays. Among the strongest driver pairs were crSetd2.crAcvr2a, crCbwd1.crNsd1, crRnf43.crAtrx, and crPten.crRasa1. FIG. 4C is a bar plot showing the number of significantly synergistic dual-crRNAs associated with each gene pair in the CCAS library (Benjamini-Hochberg adjusted p<0.05). 24 synergistic pairs were corroborated by multiple dual-crRNAs, including Brca1+Cbwd1 and Kdm6a+Trp53. FIG. 4D shows gene-level synergistic driver network based on the CCAS screen, focusing here on H2-Q2 and all first-degree connections between genes associated with H2-Q2. The complete network is shown in FIG. 12. Each node represents one gene, and each edge indicates a significant synergistic interaction (Benjamini-Hochberg adjusted p<0.05). Edge widths are scaled by SynCo score. H2-Q2 was significantly synergistic with a total of 19 other genes by this analysis, and its strongest synergistic partner was found to be Kmt2d (SynCo=8.877). FIG. 4E is a bubble chart depicting co-mutation analysis of synergistic drivers across 21 human cancer types. For each of the top 50 significant driver pairs identified through CCAS SynCo analysis, bubble dots indicate whether these gene pairs were significantly co-mutated in human cancers (where mutations are defined as nonsynonymous mutations or deep deletions). The color of each point corresponds to the average SynCo score (from mice), while the size of each point is scaled to the −log₁₀ p-value of co-mutation in each human cancer (hypergeometric test). Of all synergistic interactions identified by SynCo analysis, 132 gene combinations were significantly co-mutated in at least one cancer type (Benjamini-Hochberg adjusted p<0.05), with 46 pairs significantly co-mutated in two or more cancer types, indicating that the synergistic driver pairs identified through the mouse CCAS screen recapitulate genomic features of human cancers.

FIGS. 5A-5C are a series of plots and images illustrating a Cpf1 crRNA array library screen in a mouse model of metastasis. FIG. 5A is a schematic of the experimental approach for Cpf1 crRNA array library screen in a mouse model of metastasis to identify co-drivers of metastatic process in vivo. Lentiviral pools were generated from the CCAS plasmid library, and Cpf1⁺ KPD LCC cells subsequently infected to perform massively parallel gene-pair level mutagenesis. The mixed double mutant cell populations (CCAS-treated cells, 4×10⁶ cells per mouse, ˜400× coverage) were then injected subcutaneously into Nu/Nu mice (n=7) and Rag1−/− mice (n=4). After 8 weeks, genomic DNA was extracted from the primary tumors, four lung lobes, and other stereoscope-visible metastases, and then subjected to crRNA array sequencing. FIG. 5B is a dot-boxplot depicting the overall representation of the CCAS library across all metastasis screen samples, in terms of log₂ rpm abundance. The 3 pre-injection cell pools, as well as primary tumors and metastases from all 11 mice were sequenced. NTC-NTC controls, SKO crRNA arrays, and DKO crRNA arrays are shown. Whereas cell samples exhibited lognormal representation of the CCAS library, both primary tumors and metastases showed strong enrichment of specific SKO and DKO crRNA arrays. Notably, NTC-NTC crRNA arrays were consistently found at low abundance in all primary tumors and metastases samples. FIG. 5C shows intra-mouse Pearson correlation heatmaps of samples, showing high degree of similarity between primary tumors and metastases from the same host.

FIGS. 6A-6D are a series of plots and images illustrating enrichment analysis of crRNA arrays identified metastasis drivers and co-drivers. FIG. 6A is a violin plot showing the distribution of permutation correlations between crX.crY and crY.crX for the 4,704 DKO crRNA array combinations in the CCAS library (9,408 unique crRNA array permutations). 97.4% all crRNA array combinations were significantly correlated when comparing the two permutations associated with each combination (Benjamini-Hochberg adjusted p<0.05, by t-distribution). FIG. 6B is a volcano plot of DKO and SKO crRNA arrays compared to NTC-NTC controls in the metastasis screen. Log₂ fold change is calculated using average log₂ rpm abundance across all in vivo samples, after averaging the 3 NTC-NTC controls to get one NTC-NTC score per sample. 2933 crRNA arrays were found to be significantly enriched compared to NTC-NTC controls (Benjamini Hochberg-adjusted p<0.05), targeting 1006 gene pairs. Of these, 2813 were DKO crRNA arrays and 120 were SKO crRNA arrays. All 49 genes in the PANCAN17-mTSG CCAS library were represented within at least one significant DKO crRNA array. FIG. 6C is a bar plot of the top 15 genes ranked by the number of significant crRNA arrays associated with each gene. Arid1a, Cdh1, Kdm5c and Rb1 were the top genes associated with ≥200 independent crRNA arrays. FIG. 6D is a bar plot showing the number of significant DKO crRNA arrays associated with each gene pair in the CCAS library. Most gene pairs were represented by at least 2 independent DKO crRNA arrays. Of note, 8 gene pairs were represented by all eight crRNA arrays.

FIGS. 7A-7D are a series of plots and images illustrating modes and patterns of metastatic spread with co-drivers. Comparison of the crRNA array representations between metastases to primary tumors revealed modes of monoclonal spread (FIG. 7A) where dominant metastases in all lobes were derived from identical crRNA arrays, and polyclonal spread (FIG. 7B) where dominant metastases in all lobes were derived from several different crRNAs. FIG. 7A is an example of a monoclonal spread where all 4 lobes were dominated by a clone crNf2.crRnf43, that was also found at the primary tumor as a major clone (≥2% frequency). FIG. 7B is an example of a polyclonal spread where all 4 lobes were derived from multiple varying crRNAs. Lobe 1 was dominated by crNsd1.crNTC, which was one of major clones in the corresponding primary tumor; Lobe 2 was dominated by crH2-Q2.crCdh1, crNsd1.crAtm and crCasp8.crArid1a, which were also major clones in primary tumor. However, lobe 3 was dominated by crElf3.crFbxw7 and crRb1.crCasp8, which were not found as major clones in primary tumor; the case of lobe 4 echoes that of lobe 3 with a more complex metastatic clonal mixture, as most of its dominant clones (crBcor.crKdm5c, crAcvr2a.crNTC, crRb1.crCasp8, crCdkn2a.crApc, crApc.crKmt2b, crRasa1.crNf2, crElf3.crFbxw7 and crPten.crKdm5c) were not found as major clones in the primary tumor. FIG. 7C is a waterfall plot of enriched crRNA arrays in a metastases vs primary tumor analysis, identifying crRNA arrays that were dominant clones in metastases but not in the corresponding primary tumor. Top ranked metastasis-specific dominant crRNA arrays were found to be crCic.crKmt2b, crCdkn2a.crApc, crRasa1.crNf2, crApc.crKmt2b, crNf2.crPik3r1, crNf2.crRnf43, among 23 enriched crRNA arrays. FIG. 7D is a schematic describing several extended applications of multiplexed Cpf1 screens. The relative ease of library construction and subsequent readout with the approach described herein empowers the study of previously intractable biological problems, including combinatorial genome-wide knockout studies of synthetic lethality, as well as the discovery and characterization of epistatic networks in embryonic development and stem cell differentiation. Notably, this approach is rapidly scalable to triple knockout or higher-dimensional screens.

FIGS. 8A-8B are a series of images illustrating double knockout of Nf1 and Pten by a single crRNA array. FIG. 8A is a schematic depicting the experimental approach for testing the ability of a single crRNA array to induce mutagenesis at both Nf1 and Pten. Plasmids were designed containing a U6 promoter driving the expression of either a Pten crRNA (crPten) followed by an Nf1 crRNA (crNf1), or vice versa. Lentiviruses were subsequently generated and used to infect a tumor cell line that had been transduced with a Cpf1 expression vector (KPD.LbCpf1+). FIG. 8B shows 7 days after lentiviral infection, genomic DNA was harvested from puromycin-resistant cells for mutation analysis. Nextera library preparation and deep sequencing enabled quantitative high-resolution analysis of the mutations induced by Cpf1 activity. For each treatment condition, mutations were identified at the genomic loci targeted by crPten (left column) and by crNf1 (right column). Variant frequencies associated with each mutation are shown in the boxes to the right; for each condition, the top 5 most frequent variants are shown. The location of the protospacer adjacent motif and the crRNA are indicated at the top. Regardless of individual crRNA position within the crRNA array (top row, crPten-crNf1; bottom row, crNf1-crPten), indels were found at both Pten and Nf1 loci in KPD.LbCpf1+ cells treated with crPten-crNf1 or crNf1-crPten crRNA arrays.

FIGS. 9A-9E are a series of plots and images illustrating representation of CCAS crRNA array library in plasmid, cells, and tumors. FIG. 9A is a heatmap of pairwise Pearson correlation coefficients of crRNA array log₂ rpm abundance from CCAS plasmid library, CCAS transduced cells before transplantation (day 7 post infection), and late stage subcutaneous tumors (6.5 weeks post transplantation). Plasmid and cell samples were highly correlated with one another, while tumor samples were most correlated with other tumors. FIG. 9B is a bar plot depicting the percentage of all crRNA arrays in the CCAS library that were detected in each sample. All plasmid and cell samples contained 100% of CCAS crRNA arrays, while tumor samples exhibited significantly lower crRNA array library diversity (mean SEM=37.0% 10.5%; p=2.02 e-4 compared to plasmid and cells, t-test). FIG. 9C is a series of Q-Q plots comparing theoretical and sample quantiles of log₂ rpm crRNA array abundance in plasmid, cell, and tumor samples (cells and tumor samples averaged by group). In contrast with plasmid and cell samples, tumor samples did not appear linear on the Q-Q plot, indicating that the distribution of crRNA array abundance in plasmid and cell samples (but not tumor samples) approximated a normal distribution. FIGS. 9D-9E are a series of pie charts showing highly enriched crRNA arrays (>2% reads) across all 10 tumors; the area for each crRNA array corresponds to the percentage of reads within the tumor.

FIGS. 10A-10B are a series of plots and images illustrating outlier analysis of individual tumors compared to cells. FIG. 10A is a series of scatterplots comparing log₂ rpm abundance of crRNA arrays in individual tumors compared to cell samples (cell samples were averaged). In all tumors, crRNA arrays largely approximated a log-linear distribution, as indicated by the linear regression lines. However, there were numerous clear outliers (Bonferroni adjusted p<0.05), indicating that specific crRNA arrays had undergone positive selection in vivo. The associated regression r², coefficient, and p-value (by F-test) are noted on each plot. FIG. 10B is a barplot depicting the number of DKO and SKO outlier crRNA arrays identified within each individual tumor, as defined in FIG. 10A.

FIGS. 11A-11E are a series of plots and images illustrating crRNA array permutation has a minimal effect on enrichment. FIG. 11A is a schematic illustrating two permutations of the same crRNA array combination (crX-crY and crY-crX). To estimate possible position effects on the efficiency of Cpf1 mutagenesis, the Pearson correlation was calculated between each permutation pair in terms of log₂ rpm abundance. This value was defined as the permutation correlation. FIG. 11B is an empirical cumulative density plot of all permutation correlations across the 4,704 crRNA array combinations in the CCAS library. Greater than half of all crRNA array combinations had a correlation coefficient R≥0.97, indicating that the majority of crRNA array permutations were strongly correlated. FIG. 11C is a scatterplot comparing log₂ rpm abundance of crH2-Q2.1_crPten.240 and its permutation crPten.240_crH2-Q2.1 across all 10 tumor samples. The correlation coefficient and associated p-value of the correlation are noted in the top left (R=0.999, p=2.28 e-19). FIG. 11D is a scatterplot comparing log₂ rpm abundance of crCbwd1.84_crEpha2.5 and its permutation crEpha2.5_crCbwd.84 across all 10 tumor samples. The correlation coefficient and associated p-value of the correlation are noted in the top left (R=0.999, p=7.09 e-19). FIG. 11E shows marginal distribution meta-analysis of all 98 constituent single crRNAs in the CCAS library showing the average log₂ rpm abundance of all DKO crRNA arrays associated with each individual crRNA when present in position 1 or in position 2 of the crRNA array. The scatterplot shows the average log₂ rpm abundance for each single crRNA when in position 1 (x-axis) or position 2 (y-axis). Across all 98 single crRNAs, the average abundance for each single crRNA when in position 1 was significantly correlated with the average abundance when in position 2 (Pearson correlation coefficient (R)=0.397, p=5.25 e-5 by t-distribution), showing that individual crRNAs confer a similar selective advantage regardless of position in the crRNA array.

FIG. 12 is an image illustrating network analysis of synergistic driver pairs. The complete map of the gene-level synergistic driver network among all 49 genes in the CCAS library is shown. Each node represents one gene, and each edge indicates a statistically significant synergistic interaction between a given gene pair (Benjamini-Hochberg adjusted p-value<0.05, as in FIG. 4B). The strength of each synergistic interaction (SynCo score) is represented by edge width. Nodes are color-coded based on the degree of connectivity within the network.

FIG. 13 is a heatmap of pairwise Pearson correlation coefficients of crRNA arrays in the CCAS metastasis screen. Heatmap of pairwise Pearson correlation coefficients in log₂ rpm abundance from all 50 samples, including CCAS transduced cells before transplantation (day 7 post infection, n=3 biological replicates), primary tumors (n=11 tumors from 11 mice, 7 were Nu/Nu and 4 were Rag1−/−), and metastases (n=36 samples from 11 mice).

FIG. 14 is a heatmap illustrating the overall library representation landscape of all crRNA array abundance in the CCAS metastasis screen. Heatmap of all crRNA array abundance in log₂ rpm abundance from all 50 samples, including CCAS transduced cells before transplantation (day 7 post infection, n=3 biological replicates), primary tumors (n=11 tumors from 11 mice, 7 were Nu/Nu and 4 were Rag1−/−), and metastases (n=36 samples from 11 mice).

FIGS. 15A-15G are a series of pie charts of dominant clones in all primary tumor and metastases in the CCAS metastasis screen. Pie charts showing dominant crRNA arrays (>2% reads) in each sample, across all 11 primary tumors and 36 metastasis samples. The area for each crRNA array corresponds to the percentage of reads within the tumor.

FIG. 16 is an image illustrating a CCAS system for multiplexed genome editing in immune cells and brain endothelial cells. Arrows point to successful detection of genome editing products.

FIGS. 17A-17C illustrate the features of the pLenti-EFS-Cpf1-blast vector (SEQ ID NO: 1).

FIGS. 18A-18B illustrate the features of the pLenti-U6-DR-crRNA-puro vector (SEQ ID NO: 2).

FIGS. 19A-19B illustrate the features of the vector pSC020_pLKO_U6-Cpf1crRNA-EFS-Thy11CO-sPA (SEQ ID NO: 3).

FIGS. 20A-20F are a series of tables displaying a ranked list of putative TSGs from analysis of 17 cancer types from TCGA (PANCAN17-TSG50).

FIGS. 21A-21C illustrate Cpf1-Flip—Cre-inducible sequential mutagenesis by a single crRNA FlipArray. FIG. 21A shows schematics of vectors used in the study. The Cpf1-Flip construct contains an EFS promoter driving expression of Cpf1 and puromycin resistance, and a U6 expression cassette containing two inverted BsmbI restriction sites, flanked by a lox66 sequence and an inverted lox71 sequence. After BsmbI digestion, a crRNA FlipArray is cloned in. The FlipArray inverts upon Cre recombination, thereby switching the crRNA that is expressed. FIG. 21B is a schematic of an experimental design. Cells were first infected with lentivirus containing EFS-Cpf1-puro; U6-FlipArray. After 7 days, cells were then infected with lentivirus containing EFS-Cre to induce inversion of the FlipArray. Prior to Cre recombination, only crNf1 is expressed; following Cre recombination, crPten becomes expressed. FIG. 21C shows sequences of the FlipArray construct before and after Cre recombination. Boxes denote mutants from wildtype loxP. Prior to Cre, single mutant lox66 and lox71 sites are present. After Cre recombination, a wildtype loxP site and a double mutant lox72 site are generated.

FIGS. 22A-22K illustrate inducible sequential mutagenesis in murine cells through Cpf1-Flip. FIG. 22A is a schematic for PCR-based detection of Cre-mediated inversion of the crRNA FlipArray (Nf1 and Pten). FIG. 22B shows results from PCR-based detection of non-inverted and inverted FlipArrays at DO (n=3) and D10 (n=3) following Cre, along with input control. FIG. 22C shows quantification of gel intensities in FIG. 22B, normalized to input and expressed as a percentage of total FlipArray abundance. FIG. 22D shows detection and quantification of Cre-mediated inversion of the crRNA FlipArray at the RNA transcript level using RT-PCR (n=2 infection replicates). The expression of the inverted FlipArray was assessed at multiple timepoints following EFS-Cre infection using sequence-specific primers for the inverted FlipArray transcript as normalized to the Cpf1 mRNA level. The induction of inverted crRNA expression steadily increased through 5d after Cre. FIG. 22E shows representative Illumina targeted amplicon sequencing of the crNf1 target site in uninfected controls. No significant variants were detected. FIG. 22F shows representative Illumina targeted amplicon sequencing of the crPten target site in uninfected controls. No significant variants were detected. FIG. 22G shows representative Illumina targeted amplicon sequencing of the crNf1 target site 7 days after infection with lentivirus containing EFS-Cpf1-puro; U6-NPF-FlipArray. The top 5 most frequent variants are shown, with the associated variant frequencies in the box to the right. FIG. 22H shows representative Illumina targeted amplicon sequencing of the crPten target site 7 days after infection with lentivirus containing EFS-Cpf1-puro; U6-NPF-FlipArray. No significant variants were detected. FIG. 22I shows representative Illuminatargeted amplicon sequencing of the crNf1 target site 17 days after infection with lentivirus containing EFS-Cpf1-puro; U6-NPF-FlipArray and 10 days following EFS-Cre infection. The top 5 most frequent variants are shown, with the associated variant frequencies in the box to the right. FIG. 22J shows representative Illumina targeted amplicon sequencing of the crPten target site 17 days after infection with lentivirus containing EFS-Cpf1-puro; U6-NPF-FlipArray and 10 days following EFS-Cre infection. The top 5 most frequent variants are shown, with the associated variant frequencies in the box to the right. FIG. 22K is a dot plot detailing the total variant frequenciesat the crNf1 and crPten target sites in uninfected cells, 7 days after FlipArray transduction (−Cre), and 17 days after FlipArray transduction (+Cre). Error bars are mean s.e.m (n=2 cell replicates for uninfected group, n=3 for other conditions).

FIGS. 23A-23K illustrate inducible sequential mutagenesis in human cells through Cpf1-Flip. FIG. 23A is a schematic of a FlipArray targeting human DNMT1 and VEGFA. In the absence of Cre, crDNMT1 is expressed. Cre administration leads to the inversion of the FlipArray, leading to the expression of crVEGFA. FIG. 23B shows results from PCR-based detection of non-inverted and inverted FlipArrays at DO (n=2) and D14 (n=3) following Cre, along with the input control. FIG. 23C shows quantification of gel intensities in FIG. 23B, normalized to input and expressed as a percentage of total FlipArray abundance. FIG. 23D shows representative Illumina targeted amplicon sequencing of the crDNMT1 target site in uninfected controls. No significant variants were detected. FIG. 23E shows representative Illumina targeted amplicon sequencing of the crVEGFA target site in uninfected controls. No significant variants were detected. FIG. 23F shows representative Illumina targeted amplicon sequencing of the crDNMT1 target site 7 days after infection with lentivirus containing EFS-Cpf1-puro; U6-DVF-FlipArray. The top 5 most frequent variants are shown, with the associated variant frequencies in the box to the right. FIG. 23G shows representative Illumina targeted amplicon sequencing of the crVEGFA target site 7 days after infection with lentivirus containing EFS-Cpf1-puro; U6-DVF-FlipArray. No significant variants were detected. FIG. 23H shows representative Illumina targeted amplicon sequencing of the crDNMT1 target site 21 days after infection with lentivirus containing EFS-Cpf1-puro; U6-DVF-FlipArray and 14 days following EFS-Cre infection. The top 5 most frequent variants are shown, with the associated variant frequencies in the box to the right. FIG. 23I shows representative Illumina targeted amplicon sequencing of the crVEGFA target site 21 days after infection with lentivirus containing EFS-Cpf1-puro; U6-DVF-FlipArray and 14 days following EFS-Cre infection. The associated variant frequencies are shown in the box to the right. FIGS. 23J-23K are dot plots detailing the total variant frequencies at the crDNMT1 and crVEGFA target sites in uninfected cells, 7 days after FlipArray transduction (−Cre), and 21 days after FlipArray transduction (+Cre). Error bars are mean s.e.m (n=2 cell replicates for uninfected and D7 conditions, n=6 for D21 timepoint).

FIGS. 24A-24C illustrate pooled sequential mutagenesis to model acquired resistance to immunotherapy. FIG. 24A is a schematic of the experimental approach for pooled sequential mutagenesis using Cpf1-Flip. Following restriction digest, a library of FlipArrays is cloned into the base vector. In each FlipArray, the first crRNA targets a tumor suppressor (Nf1), while the second crRNA targets a panel of putative immunomodulatory factors. Cre-mediated inversion induces expression of the second crRNA. FIG. 24B is a dot plot detailing the total variant frequencies at the crNf1 target site in uninfected cells, 14 days after FlipArray transduction (−Cre), and 28 days after FlipArray transduction (+Cre). Error bars are mean s.e.m (n=3 cell replicates for all conditions). FIG. 24C is a dot plot detailing the total variant frequencies at the second crRNA target sites (Fas1, Ido1, Jak2, Lgals9, B2m, and Cd274) in uninfected cells, 14 days after FlipArray transduction (−Cre), and 28 days after FlipArray transduction (+Cre). Error bars are mean s.e.m (n=3 cell replicates for all conditions).

FIGS. 25A-25B illustrate applications and variations of Cpf1-Flip. FIG. 25A is a schematic of several variations of Cpf1-Flip, using modified Cpf1 effector proteins. Sequential gene activation, gene repression, and epigenetic modification can all be readily performed using Cpf1-Flip. FIG. 25B illustrates Cpf1-Flip applied to model the evolution of cancer in a direct in vivo system. Since Cpf1-Flip operates in a stepwise manner, it is possible to temporally separate the initial mutagenesis event (in this case targeting a tumor suppressor gene, or TSG). After tumorigenesis, induction of FlipArray inversion activates the second set of crRNAs, allowing for parallel interrogation of clonal dynamics in vivo.

FIGS. 26A-26C illustrate evaluation of in vivo library diversity in the absence of mutagenesis. FIG. 26A shows the experimental design used to evaluate the suitability of the in vivo transplant model for high-throughput genetic interrogation. To model neutral selection in the absence of mutagenesis, a lentiviral library containing random 8 mer barcodes was introduced into KPD cells, for a theoretical total of 48=65,536 unique barcodes. 4×10⁶ cells were then injected into mice to mimic normal experimental conditions. After 12 days, genomic DNA was extracted from the nodules for barcode sequencing and assessment of library diversity. FIG. 26B is a bar plot detailing the percentage of all possible 8 mers that were recovered in each sample (cell pool, n=1; nu/nu mice, n=2; Rag^{-/-} mice, n=4). FIG. 26C is a scatter-box plot of the abundances of all possible 8 mers in cell pools, nu/nu mice, and Rag^{-/-} mice.

FIGS. 27A-27E illustrate interrogation of metastasis driver combinations by massively-parallel Cpf1-crRNA array profiling (MCAP). FIG. 27 is a schematic describing the design and synthesis of a library for massively-parallel Cpf1-crRNA array profiling (MCAP) of metastasis driver combinations. The top 23 tumor suppressors (TSGs) were identified from a human metastasis genomics cohort (MET-500), as well as the top 3 hits from a prior single-gene mouse metastasis screen (total n=26 genes). 4 crRNAs were chosen for each gene. Along with 52 NTC-NTC control crRNA arrays, a library was designed containing 1,326 NTC-NTC arrays, 5,408 single knockout (SKO) arrays targeting 26 single genes, and 5200 double knockout (DKO) arrays targeting 325 gene pairs for a total of 11,934 crRNA arrays (MCAP-MET library). FIG. 27B shows an experimental design for combinatorial interrogation of metastasis drivers in vivo. After generation of the MCAP-MET library, 4×10⁶ Cpf1+ KPD cells were transduced and then injected into nu/nu mice. 6 weeks after injection, primary tumors and lung lobes were harvested for genomic DNA extraction and crRNA array sequencing. FIG. 27C is a density plot showing the distribution of MCAP-MET library abundance in terms of log₂ reads per million (rpm). All crRNA arrays were detected in the plasmid library, following a log-normal distribution of abundances. FIG. 27D is a density plot of the number of unique barcodes associated with each crRNA array. A total of 774,296 unique barcoded-crRNA arrays (BC-arrays) were detected in the MCAP-MET plasmid library. FIG. 27E is a scatter plot of the normalized MCAP-MET library abundance in plasmid and averaged cell pools. Data are shown in terms of log₂ reads per million (rpm). The linear regression line for the entire MCAP-MET library is overlaid, demonstrating high concordance between plasmid library and cell pools. Shading on the regression line denotes the 95% confidence interval (CI).

FIG. 28 illustrates barcode-level analysis of the MCAP-MET library. Empirical CDF of the abundance of all detected barcoded-crRNA arrays in the MCAP-MET library (left), and a violin plot of the abundances (right).

FIGS. 29A-29B illustrate representation of MCAP-MET crRNA array library in plasmid, cells, primary tumors, and lung metastases. FIG. 29A is a heat map of pairwise Spearman correlation coefficients of crRNA array log₂ rpm abundance from MCAP-MET plasmid library, MCAP transduced cells before transplantation (day 7 or day 14 post infection), primary tumors, and lung metastases. Plasmid and cell samples were highly correlated with one another. FIG. 29B is a box-dot plot of crRNA array log₂ rpm abundance from MCAP-MET profiling experiment of all samples, including plasmid library, MCAP transduced cells before transplantation (day 7 or day 14 post infection), primary tumors, and lung metastases.

FIGS. 30A-30L illustrate clonal compositions and crRNA array enrichment analysis. FIG. 30A is a bar plot of the number of clones present at ≥0.001% frequency (1 in 10,000) in cell pools (light gray), primary tumors (*) and lung metastases. Sample annotations are noted below. FIG. 30B is a Violin plot of the number of clones present at ≥0.001% frequency in cell pools, primary tumors, and lung metastases. Cells vs. primary tumors (Wilcoxon rank sum test, p=0.0002), cells vs. lung metastases (p=0.0001), and primary tumors vs. lung metastases (p=0.0162). FIG. 30C is a dot plot of the relative frequencies of clones at ≥0.001% frequency across cell pools, primary tumors, and lung metastases. Relative frequencies are expressed as percentages of total reads in each sample. Points are colored by cell sample/mouse ID. FIG. 30D shows empirical CDF of all clones at ≥0.001% frequency in cell pools, primary tumors (*) and lung metastases (**), expressed as percentages of total reads in each sample. The clone size distributions in primary tumors and lung metastases were significantly different (Kolmogorov-Smirnov test, p<2.2*10⁻¹⁶). FIG. 30E is a Venn diagram of gene pairs that were enriched in ≥50% of primary tumors or lung metastases. FIG. 30F is a histogram detailing the percentage of independent crRNA arrays that were enriched in primary tumors for each single gene (left) or gene pair (right). FIG. 30G is a table of the top genes/gene pairs in terms of the percentage of independent crRNA arrays that were enriched in primary tumors. Colors correspond to the histograms in FIG. 30F. FIG. 30H is a histogram detailing the percentage of independent crRNA arrays that were enriched in lung metastases for each single gene (left) or gene pair (right). FIG. 30I is a Table of the top genes/gene pairs in terms of the percentage of independent crRNA arrays that were enriched in lung metastases. Colors correspond to the histograms in FIG. 30H. FIGS. 30J-30L are enrichment bar plots of multiple independent crRNA arrays targeting Nf2_Rb1 (FIG. 30J), Nf2_Pten (FIG. 30K), and Nf2_Trim72 (FIG. 30L) in lung metastases.

FIGS. 31A-31F illustrate analysis of large clones in primary tumors and lung metastases. FIG. 31A is a bar plot of the number of clones present at ≥0.01% frequency in primary tumors (*) and lung metastases. Mouse IDs are annotated below. Noted that cell samples do not have clones passing this frequency cutoff due to the high diversity in the population. FIG. 31B is a Violin plot of the number of clones present at ≥0.01% frequency in primary tumors and lung metastases. Collectively, primary tumors had significantly more clones at ≥0.01% frequency than lung metastases (Wilcoxon rank sum test, p<0.0023). FIG. 31C is a dot plot of the relative frequencies of clones at ≥0.01% frequency across primary tumors and lung metastases. Relative frequencies are expressed as percentages of total reads in each sample. FIG. 31D shows empirical CDF of all clones at ≥0.01% frequency in primary tumors (*) and lung metastases, expressed as percentages of total reads in each sample. The clone size distributions in primary tumors and lung metastases were significantly different (Kolmogorov-Smirnov test, p=0.0412).

FIG. 31E is a Violin plot of Shannon diversity indices in primary tumors and lung metastases for clones at ≥0.01% frequency. Primary tumors were significantly more diverse with regard to clone frequency distribution (Wilcoxon rank sum test, p=0.0183). FIG. 31F is a Violin plot of Shannon diversity indices in cell pools, primary tumors, and lung metastases for clones at ≥0.001% frequency. Cells vs. primary tumors (Wilcoxon rank sum test, p=0.0002), cells vs. lung metastases (p=3.28*10-), and primary tumors vs. lung metastases (p=0.0212).

FIGS. 32A-32F illustrate identification of mutation combinations with enhanced metastatic potential. FIGS. 32A, 32C, 32E are scatter plots of MCAP-MET crRNA array abundance in cell pools vs. primary tumors (FIG. 32A), cell pools vs. lung metastases (FIG. 32C), and primary tumors vs. lung metastases (FIG. 32E). Data are shown in terms of average log₂ reads per million (rpm) across the indicated sample type. To illustrate the null distribution, the linear regression line of NTC-NTC control arrays is overlain. Shading on the regression line denotes the 95% CI. FIGS. 32B, 32D, 32F are scatter plots of MCAP-MET single gene and gene pair abundance in cell pools vs. primary tumors (FIG. 32B), cell pools vs. lung metastases (FIG. 32D), and primary tumors vs. lung metastases (FIG. 32F). Data are shown in terms of average log₂ rpm across the indicated sample type, after first averaging the constituent crRNA arrays for each gene/gene pair. The linear regression was calculated over the entire library, with the 95% CI shaded in. Single genes and gene pairs that were found to be significant outliers are outlined and enlarged, with s.e.m. error bars.

FIGS. 33A-33I illustrate identification of synergistic mutation combinations. FIG. 33A is a schematic of the analytical workflow to identify synergistic mutation combinations. crRNA array abundances were averaged to the corresponding gene/gene pair, then compared across samples. To identify synergistic gene pairs, a synergy coefficient score (SynCo) was also calculated. For a given gene pair NM, the SynCo is defined as DKO_{NM}−SKO_{N}−SKO_{M} using median values across the sample cohort. A positive SynCo value indicates the selective advantage of the gene pair is greater than that of the two individual genes combined. FIG. 33B is a scatter plot of the −log₁₀ p-values for each gene pair (Wilcox rank sum test), compared to the constituent single genes. Synergistic gene pairs are labeled. FIG. 33C is a scatter plot of the median differential abundance for each gene pair compared to the constituent single genes. Synergistic gene pairs are labeled with the. FIGS. 33D-33I are boxplots detailing the log₂ rpm abundances of the indicated genotypes, with associated Wilcoxon rank sum p-values and SynCo scores noted. Statistics are in reference to the DKO genotype. Nf2/Trim72 (FIG. 33D), Chd1/Nf2 (FIG. 33E), Chd1/Kmt2d (FIG. 33F), Jak1/Kmt2c (FIG. 33G), Kmt2d/Pten (FIG. 33H), and Nf1Pten (FIG. 33I).

FIG. 34 illustrates relative selective advantages of gene pair vs. single gene knockouts. Heat map of the change in log₂ rpm abundance in lung metastases for each single gene knockout, relative to the indicated second knockout. A positive value means that the second knockout (rows) granted a relative selective advantage to the reference knockout (columns), while a negative value means the second knockout was relatively disadvantageous compared to the single knockout.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, specific materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20% or 10%, more preferably 5%, even more preferably 1%, and still more preferably 0.1% from the specified value, as such variations are appropriate to perform the disclosed methods.

As used herein the term “amount” refers to the abundance or quantity of a constituent in a mixture.

As used herein, the term “bp” refers to base pair.

The term “complementary” refers to the degree of anti-parallel alignment between two nucleic acid strands. Complete complementarity requires that each nucleotide be across from its opposite. No complementarity requires that each nucleotide is not across from its opposite. The degree of complementarity determines the stability of the sequences to be together or anneal/hybridize. Furthermore various DNA repair functions as well as regulatory functions are based on base pair complementarity.

The term “CRISPR/Cas” or “clustered regularly interspaced short palindromic repeats” or “CRISPR” refers to DNA loci containing short repetitions of base sequences followed by short segments of spacer DNA from previous exposures to a virus or plasmid. Bacteria and archaea have evolved adaptive immune defenses termed CRISPR/CRISPR-associated (Cas) systems that use short RNA to direct degradation of foreign nucleic acids. In bacteria, the CRISPR system provides acquired immunity against invading foreign DNA via RNA-guided DNA cleavage. “crRNA” or “CRISPR targeting RNA” is the transcribed region of the unique “spacer” sequences found in CRISPRs. The cRNAs confer target specificity to the endonuclease, e.g. Cpf1.

The term “cleavage” refers to the breakage of covalent bonds, such as in the backbone of a nucleic acid molecule or the hydrolysis of peptide bonds. Cleavage can be initiated by a variety of methods, including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. DNA cleavage can result in the production of either blunt ends or staggered ends. In certain embodiments, fusion polypeptides can be used for targeting cleaved double-stranded DNA.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “knockdown” as used herein refers to a decrease in gene expression of one or more genes. The term “knockout” as used herein refers to the ablation of gene expression of one or more genes.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

A “mutation” as used herein is a change in a DNA sequence resulting in an alteration from a given reference sequence (which may be, for example, an earlier collected DNA sample from the same subject). The mutation can comprise deletion and/or insertion and/or duplication and/or substitution of at least one deoxyribonucleic acid base such as a purine (adenine and/or thymine) and/or a pyrimidine (guanine and/or cytosine). Mutations may or may not produce discernible changes in the observable characteristics (phenotype) of an organism (subject).

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T”.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides.

Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means. Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

A “sample” or “biological sample” as used herein means a biological material from a subject, including but is not limited to organ, tissue, exosome, blood, plasma, saliva, urine and other body fluid. A sample can be any source of material obtained from a subject.

As used herein, the terms “sequencing” or “nucleotide sequencing” refer to determining the order of nucleotides (base sequences) in a nucleic acid sample, e.g. DNA or RNA. Many techniques are available such as Sanger sequencing and high-throughput sequencing technologies (also known as next-generation sequencing technologies) such as Illumina's HiSeq and MiSeq platforms or the GS FLX platform offered by Roche Applied Science.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

A “target site” or “target sequence” refers to a genomic nucleic acid sequence that defines a portion of a nucleic acid to which a binding molecule may specifically bind under conditions sufficient for binding to occur.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one that has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention provides, in one aspect, compositions and methods for simultaneously mutagenizing multiple target sequences in a cell. In certain aspects, the invention provides compositions and methods for sequentially mutagenizing multiple target sequences in a cell. In other aspects, the invention provides methods for identifying synergistic drivers of transformation and/or tumorigenesis and/or metastasis. In other aspects, the invention provides in vivo methods for identifying and mapping genetic interactions.

### Compositions

Certain aspects of the invention include lentiviral vectors for use in genome editing. In one aspect, the invention includes a vector comprising a first long terminal repeat (LTR) sequence, an Embryonal Fyn-Associated Substrate (EFS) sequence, a Cpf1 sequence, a Nuclear Localization Signal (NLS) sequence, a Flag2A sequence, an antibiotic resistance sequence, and a second LTR sequence (pLenti-EFS-Cpf1-blast vector, LentiCpf1 for short). The Cpf1 enzyme can be derived from any genera of microbes including but not limited to Parcubacteria, Lachnospiraceae, Butyrivibrio, Peregrinibacteria, Acidaminococcus, Porphyromonas, Lachnospiraceae, Porphromonas, Prevotella, Moraxela, Smithella, Leptospira, Lachnospiraceae, Francisella, Candidatus, and Eubacterium. In certain embodiments, Cpf1 is derived from a species from the Lachnospiraceae genus (LbCpf1). In some embodiments, the Cpf1 sequence comprises a humanized form of a Lachnospiraceae bacterium Cpf1 (LbCpf1). In one embodiment, the antibiotic resistance sequence is a blasticidin resistance sequence. In one embodiment, the vector comprises SEQ ID NO: 1 (FIGS. 17A-17C).

```
pLenti-EFS-Cpf1-blast vector (SEQ ID NO: 1):
        1 gtcgacggat cgggagatct cccgatcccc tatggtgcac tctcagtaca atctgctctg
      61 atgccgcata gttaagccag tatctgctcc ctgcttgtgt gttggaggtc gctgagtagt
    121 gcgcgagcaa aatttaagct acaacaaggc aaggcttgac cgacaattgc atgaagaatc
    181 tgcttagggt taggcgtttt gcgctgcttc gcgatgtacg ggccagatat acgcgttgac
    241 attgattatt gactagttat taatagtaat caattacggg gtcattagtt catagcccat
    301 atatggagtt ccgcgttaca taacttacgg taaatggccc gcctggctga ccgcccaacg
    361 acccccgccc attgacgtca ataatgacgt atgttcccat agtaacgcca atagggactt
    421 tccattgacg tcaatgggtg gagtatttac ggtaaactgc ccacttggca gtacatcaag
    481 tgtatcatat gccaagtacg ccccctattg acgtcaatga cggtaaatgg cccgcctggc
    541 attatgccca gtacatgacc ttatgggact ttcctacttg gcagtacatc tacgtattag
    601 tcatcgctat taccatggtg atgcggtttt ggcagtacat caatgggcgt ggatagcggt
    661 ttgactcacg gggatttcca agtctccacc ccattgacgt caatgggagt ttgttttggc
    721 accaaaatca acgggacttt ccaaaatgtc gtaacaactc cgccccattg acgcaaatgg
    781 gcggtaggcg tgtacggtgg gaggtctata taagcagcgc gttttgcctg tactgggtct
    841 ctctggttag accagatctg agcctgggag ctctctggct aactagggaa cccactgctt
    901 aagcctcaat aaagcttgcc ttgagtgctt caagtagtgt gtgcccgtct gttgtgtgac
    961 tctggtaact agagatccct cagacccttt tagtcagtgt ggaaaatctc tagcagtggc
  1021 gcccgaacag ggacttgaaa gcgaaaggga aaccagagga gctctctcga cgcaggactc
  1081 ggcttgctga agcgcgcacg gcaagaggcg aggggcggcg actggtgagt acgccaaaaa
  1141 ttttgactag cggaggctag aaggagagag atgggtgcga gagcgtcagt attaagcggg
  1201 ggagaattag atcgcgatgg gaaaaaattc ggttaaggcc agggggaaag aaaaaatata
  1261 aattaaaaca tatagtatgg gcaagcaggg agctagaacg attcgcagtt aatcctggcc
  1321 tgttagaaac atcagaaggc tgtagacaaa tactgggaca gctacaacca tcccttcaga
  1381 caggatcaga agaacttaga tcattatata atacagtagc aaccctctat tgtgtgcatc
  1441 aaaggataga gataaaagac accaaggaag ctttagacaa gatagaggaa gagcaaaaca
  1501 aaagtaagac caccgcacag caagcggccg ctgatcttca gacctggagg aggagatatg
  1561 agggacaatt ggagaagtga attatataaa tataaagtag taaaaattga accattagga
  1621 gtagcaccca ccaaggcaaa gagaagagtg gtgcagagag aaaaaagagc agtgggaata
  1681 ggagctttgt tccttgggtt cttgggagca gcaggaagca ctatgggcgc agcgtcaatg
  1741 acgctgacgg tacaggccag acaattattg tctggtatag tgcagcagca gaacaatttg
  1801 ctgagggcta ttgaggcgca acagcatctg ttgcaactca cagtctgggg catcaagcag
  1861 ctccaggcaa gaatcctggc tgtggaaaga tacctaaagg atcaacagct cctggggatt
  1921 tggggttgct ctggaaaact catttgcacc actgctgtgc cttggaatgc tagttggagt
  1981 aataaatctc tggaacagat ttggaatcac acgacctgga tggagtggga cagagaaatt
  2041 aacaattaca caagcttaat acactcctta attgaagaat cgcaaaacca gcaagaaaag
  2101 aatgaacaag aattattgga attagataaa tgggcaagtt tgtggaattg gtttaacata
  2161 acaaattggc tgtggtatat aaaattattc ataatgatag taggaggctt ggtaggttta
  2221 agaatagttt ttgctgtact ttctatagtg aatagagtta ggcagggata ttcaccatta
  2281 tcgtttcaga cccacctccc aaccccgagg ggacccgaca ggcccgaagg aatagaagaa
  2341 gaaggtggag agagagacag agacagatcc attcgattag tgaacggatc ggcactgcgt
  2401 gcgccaattc tgcagacaaa tggcagtatt catccacaat tttaaaagaa aaggggggat
  2461 tggggggtac agtgcagggg aaagaatagt agacataata gcaacagaca tacaaactaa
  2521 agaattacaa aaacaaatta caaaaattca aaattttcgg gtttattaca gggacagcag
  2581 agatccagtt tggttaatta aTCGAGTGGC TCCGGTGCCC GTCAGTGGGC AGAGCGCACA
  2641 TCGCCCACAG TCCCCGAGAA GTTGGGGGGA GGGGTCGGCA ATTGAACCGG TGCCTAGAGA
  2701 AGGTGGCGCG GGGTAAACTG GGAAAGTGAT GTCGTGTACT GGCTCCGCCT TTTTCCCGAG
  2761 GGTGGGGGAG AACCGTATAT AAGTGCAGTA GTCGCCGTGA ACGTTCTTTT TCGCAACGGG
  2821 TTTGCCGCCA GAACACAGGT GTCGTGACGC GGGATCCATG AGCAAGCTGG AGAAGTTTAC
  2881 AAACTGCTAC TCCCTGTCTA AGACCCTGAG GTTCAAGGCC ATCCCTGTGG GCAAGACCCA
  2941 GGAGAACATC GACAATAAGC GGCTGCTGGT GGAGGACGAG AAGAGAGCCG AGGATTATAA
  3001 GGGCGTGAAG AAGCTGCTGG ATCGCTACTA TCTGTCTTTT ATCAACGACG TGCTGCACAG
  3061 CATCAAGCTG AAGAATCTGA ACAATTACAT CAGCCTGTTC CGGAAGAAAA CCAGAACCGA
  3121 GAAGGAGAAT AAGGAGCTGG AGAACCTGGA GATCAATCTG CGGAAGGAGA TCGCCAAGGC
  3181 CTTCAAGGGC AACGAGGGCT ACAAGTCCCT GTTTAAGAAG GATATCATCG AGACAATCCT
  3241 GCCAGAGTTC CTGGACGATA AGGACGAGAT CGCCCTGGTG AACAGCTTCA ATGGCTTTAC
  3301 CACAGCCTTC ACCGGCTTCT TTGATAACAG AGAGAATATG TTTTCCGAGG AGGCCAAGAG
  3361 CACATCCATC GCCTTCAGGT GTATCAACGA GAATCTGACC CGCTACATCT CTAATATGGA
  3421 CATCTTCGAG AAGGTGGACG CCATCTTTGA TAAGCACGAG GTGCAGGAGA TCAAGGAGAA
  3481 GATCCTGAAC AGCGACTATG ATGTGGAGGA TTTCTTTGAG GGCGAGTTCT TTAACTTTGT
  3541 GCTGACACAG GAGGGCATCG ACGTGTATAA CGCCATCATC GGCGGCTTCG TGACCGAGAG
  3601 CGGCGAGAAG ATCAAGGGCC TGAACGAGTA CATCAACCTG TATAATCAGA AAACCAAGCA
  3661 GAAGCTGCCT AAGTTTAAGC CACTGTATAA GCAGGTGCTG AGCGATCGGG AGTCTCTGAG
  3721 CTTCTACGGC GAGGGCTATA CATCCGATGA GGAGGTGCTG GAGGTGTTTA GAAACACCCT
  3781 GAACAAGAAC AGCGAGATCT TCAGCTCCAT CAAGAAGCTG GAGAAGCTGT TCAAGAATTT
  3841 TGACGAGTAC TCTAGCGCCG GCATCTTTGT GAAGAACGGC CCCGCCATCA GCACAATCTC
  3901 CAAGGATATC TTCGGCGAGT GGAACGTGAT CCGGGACAAG TGGAATGCCG AGTATGACGA
  3961 TATCCACCTG AAGAAGAAGG CCGTGGTGAC CGAGAAGTAC GAGGACGATC GGAGAAAGTC
  4021 CTTCAAGAAG ATCGGCTCCT TTTCTCTGGA GCAGCTGCAG GAGTACGCCG ACGCCGATCT
  4081 GTCTGTGGTG GAGAAGCTGA AGGAGATCAT CATCCAGAAG GTGGATGAGA TCTACAAGGT
  4141 GTATGGCTCC TCTGAGAAGC TGTTCGACGC CGATTTTGTG CTGGAGAAGA GCCTGAAGAA
  4201 GAACGACGCC GTGGTGGCCA TCATGAAGGA CCTGCTGGAT TCTGTGAAGA GCTTCGAGAA
  4261 TTACATCAAG GCCTTCTTTG GCGAGGGCAA GGAGACAAAC AGGGACGAGT CCTTCTATGG
  4321 CGATTTTGTG CTGGCCTACG ACATCCTGCT GAAGGTGGAC CACATCTACG ATGCCATCCG
  4381 CAATTATGTG ACCCAGAAGC CCTACTCTAA GGATAAGTTC AAGCTGTATT TTCAGAACCC
  4441 TCAGTTCATG GGCGGCTGGG ACAAGGATAA GGAGACAGAC TATCGGGCCA CCATCCTGAG
  4501 ATACGGCTCC AAGTACTATC TGGCCATCAT GGATAAGAAG TACGCCAAGT GCCTGCAGAA
  4561 GATCGACAAG GACGATGTGA ACGGCAATTA CGAGAAGATC AACTATAAGC TGCTGCCCGG
  4621 CCCTAATAAG ATGCTGCCAA AGGTGTTCTT TTCTAAGAAG TGGATGGCCT ACTATAACCC
  4681 CAGCGAGGAC ATCCAGAAGA TCTACAAGAA TGGCACATTC AAGAAGGGCG ATATGTTTAA
  4741 CCTGAATGAC TGTCACAAGC TGATCGACTT CTTTAAGGAT AGCATCTCCC GGTATCCAAA
  4801 GTGGTCCAAT GCCTACGATT TCAACTTTTC TGAGACAGAG AAGTATAAGG ACATCGCCGG
  4861 CTTTTACAGA GAGGTGGAGG AGCAGGGCTA TAAGGTGAGC TTCGAGTCTG CCAGCAAGAA
  4921 GGAGGTGGAT AAGCTGGTGG AGGAGGGCAA GCTGTATATG TTCCAGATCT ATAACAAGGA
  4981 CTTTTCCGAT AAGTCTCACG GCACACCCAA TCTGCACACC ATGTACTTCA AGCTGCTGTT
  5041 TGACGAGAAC AATCACGGAC AGATCAGGCT GAGCGGAGGA GCAGAGCTGT TCATGAGGCG
  5101 CGCCTCCCTG AAGAAGGAGG AGCTGGTGGT GCACCCAGCC AACTCCCCTA TCGCCAACAA
  5161 GAATCCAGAT AATCCCAAGA AAACCACAAC CCTGTCCTAC GACGTGTATA AGGATAAGAG
  5221 GTTTTCTGAG GACCAGTACG AGCTGCACAT CCCAATCGCC ATCAATAAGT GCCCCAAGAA
  5281 CATCTTCAAG ATCAATACAG AGGTGCGCGT GCTGCTGAAG CACGACGATA ACCCCTATGT
  5341 GATCGGCATC GATAGGGGCG AGCGCAATCT GCTGTATATC GTGGTGGTGG ACGGCAAGGG
  5401 CAACATCGTG GAGCAGTATT CCCTGAACGA GATCATCAAC AACTTCAACG GCATCAGGAT
  5461 CAAGACAGAT TACCACTCTC TGCTGGACAA GAAGGAGAAG GAGAGGTTCG AGGCCCGCCA
  5521 GAACTGGACC TCCATCGAGA ATATCAAGGA GCTGAAGGCC GGCTATATCT CTCAGGTGGT
  5581 GCACAAGATC TGCGAGCTGG TGGAGAAGTA CGATGCCGTG ATCGCCCTGG AGGACCTGAA
  5641 CTCTGGCTTT AAGAATAGCC GCGTGAAGGT GGAGAAGCAG GTGTATCAGA AGTTCGAGAA
  5701 GATGCTGATC GATAAGCTGA ACTACATGGT GGACAAGAAG TCTAATCCTT GTGCAACAGG
  5761 CGGCGCCCTG AAGGGCTATC AGATCACCAA TAAGTTCGAG AGCTTTAAGT CCATGTCTAC
  5821 CCAGAACGGC TTCATCTTTT ACATCCCTGC CTGGCTGACA TCCAAGATCG ATCCATCTAC
  5881 CGGCTTTGTG AACCTGCTGA AAACCAAGTA TACCAGCATC GCCGATTCCA AGAAGTTCAT
  5941 CAGCTCCTTT GACAGGATCA TGTACGTGCC CGAGGAGGAT CTGTTCGAGT TTGCCCTGGA
  6001 CTATAAGAAC TTCTCTCGCA CAGACGCCGA TTACATCAAG AAGTGGAAGC TGTACTCCTA
  6061 CGGCAACCGG ATCAGAATCT TCCGGAATCC TAAGAAGAAC AACGTGTTCG ACTGGGAGGA
  6121 GGTGTGCCTG ACCAGCGCCT ATAAGGAGCT GTTCAACAAG TACGGCATCA ATTATCAGCA
  6181 GGGCGATATC AGAGCCCTGC TGTGCGAGCA GTCCGACAAG GCCTTCTACT CTAGCTTTAT
  6241 GGCCCTGATG AGCCTGATGC TGCAGATGCG GAACAGCATC ACAGGCCGCA CCGACGTGGA
  6301 TTTTCTGATC AGCCCTGTGA AGAACTCCGA CGGCATCTTC TACGATAGCC GGAACTATGA
  6361 GGCCCAGGAG AATGCCATCC TGCCAAAGAA CGCCGACGCC AATGGCGCCT ATAACATCGC
  6421 CAGAAAGGTG CTGTGGGCCA TCGGCCAGTT CAAGAAGGCC GAGGACGAGA AGCTGGATAA
  6481 GGTGAAGATC GCCATCTCTA ACAAGGAGTG GCTGGAGTAC GCCCAGACCA GCGTGAAGCA
  6541 CAAAAGGCCG GCGGCCACGA AAAAGGCCGG CCAGGCAAAA AAGAAAAAGG ATTACAAAGA
  6601 CGATGACGAT AAGGGCAGCG GCGCCACCAA CTTCAGCCTG CTGAAGCAGG CCGGCGACGT
  6661 GGAGGAGAAC CCCGGCCCCa tggccaagcc tttgtctcaa gaagaatcca ccctcattga
  6721 aagagcaacg gctacaatca acagcatccc catctctgaa gactacagcg tcgccagcgc
  6781 agctctctct agcgacggcc gcatcttcac tggtgtcaat gtatatcatt ttactggggg
  6841 accttgtgca gaactcgtgg tgctgggcac tgctgctgct gcggcagctg gcaacctgac
  6901 ttgtatcgtc gcgatcggaa atgagaacag gggcatcttg agcccctgcg gacggtgccg
  6961 acaggtgctt ctcgatctgc atcctgggat caaagccata gtgaaggaca gtgatggaca
  7021 gccgacggca gttgggattc gtgaattgct gccctctggt tatgtgtggg agggctaaga
  7081 attcgatatc aagcttatcg ataatcaacc tctggattac aaaatttgtg aaagattgac
  7141 tggtattctt aactatgttg ctccttttac gctatgtgga tacgctgctt taatgccttt
  7201 gtatcatgct attgcttccc gtatggcttt cattttctcc tccttgtata aatcctggtt
  7261 gctgtctctt tatgaggagt tgtggcccgt tgtcaggcaa cgtggcgtgg tgtgcactgt
  7321 gtttgctgac gcaaccccca ctggttgggg cattgccacc acctgtcagc tcctttccgg
  7381 gactttcgct ttccccctcc ctattgccac ggcggaactc atcgccgcct gccttgcccg
  7441 ctgctggaca ggggctcggc tgttgggcac tgacaattcc gtggtgttgt cggggaaatc
  7501 atcgtccttt ccttggctgc tcgcctgtgt tgccacctgg attctgcgcg ggacgtcctt
  7561 ctgctacgtc ccttcggccc tcaatccagc ggaccttcct tcccgcggcc tgctgccggc
  7621 tctgcggcct cttccgcgtc ttcgccttcg ccctcagacg agtcggatct ccctttgggc
  7681 cgcctccccg catcgatacc gtcgacctcg agacctagaa aaacatggag caatcacaag
  7741 tagcaataca gcagctacca atgctgattg tgcctggcta gaagcacaag aggaggagga
  7801 ggtgggtttt ccagtcacac ctcaggtacc tttaagacca atgacttaca aggcagctgt
  7861 agatcttagc cactttttaa aagaaaaggg gggactggaa gggctaattc actcccaacg
  7921 aagacaagat atccttgatc tgtggatcta ccacacacaa ggctacttcc ctgattggca
  7981 gaactacaca ccagggccag ggatcagata tccactgacc tttggatggt gctacaagct
  8041 agtaccagtt gagcaagaga aggtagaaga agccaatgaa ggagagaaca cccgcttgtt
  8101 acaccctgtg agcctgcatg ggatggatga cccggagaga gaagtattag agtggaggtt
  8161 tgacagccgc ctagcatttc atcacatggc ccgagagctg catccggact gtactgggtc
  8221 tctctggtta gaccagatct gagcctggga gctctctggc taactaggga acccactgct
  8281 taagcctcaa taaagcttgc cttgagtgct tcaagtagtg tgtgcccgtc tgttgtgtga
  8341 ctctggtaac tagagatccc tcagaccctt ttagtcagtg tggaaaatct ctagcagggc
  8401 ccgtttaaac ccgctgatca gcctcgactg tgccttctag ttgccagcca tctgttgttt
  8461 gcccctcccc cgtgccttcc ttgaccctgg aaggtgccac tcccactgtc ctttcctaat
  8521 aaaatgagga aattgcatcg cattgtctga gtaggtgtca ttctattctg gggggtgggg
  8581 tggggcagga cagcaagggg gaggattggg aagacaatag caggcatgct ggggatgcgg
  8641 tgggctctat ggcttctgag gcggaaagaa ccagctgggg ctctaggggg tatccccacg
  8701 cgccctgtag cggcgcatta agcgcggcgg gtgtggtggt tacgcgcagc gtgaccgcta
  8761 cacttgccag cgccctagcg cccgctcctt tcgctttctt cccttccttt ctcgccacgt
  8821 tcgccggctt tccccgtcaa gctctaaatc gggggctccc tttagggttc cgatttagtg
  8881 ctttacggca cctcgacccc aaaaaacttg attagggtga tggttcacgt agtgggccat
  8941 cgccctgata gacggttttt cgccctttga cgttggagtc cacgttcttt aatagtggac
  9001 tcttgttcca aactggaaca acactcaacc ctatctcggt ctattctttt gatttataag
  9061 ggattttgcc gatttcggcc tattggttaa aaaatgagct gatttaacaa aaatttaacg
  9121 cgaattaatt ctgtggaatg tgtgtcagtt agggtgtgga aagtccccag gctccccagc
  9181 aggcagaagt atgcaaagca tgcatctcaa ttagtcagca accaggtgtg gaaagtcccc
  9241 aggctcccca gcaggcagaa gtatgcaaag catgcatctc aattagtcag caaccatagt
  9301 cccgccccta actccgccca tcccgcccct aactccgccc agttccgccc attctccgcc
  9361 ccatggctga ctaatttttt ttatttatgc agaggccgag gccgcctctg cctctgagct
  9421 attccagaag tagtgaggag gcttttttgg aggcctaggc ttttgcaaaa agctcccggg
  9481 agcttgtata tccattttcg gatctgatca gcacgtgttg acaattaatc atcggcatag
  9541 tatatcggca tagtataata cgacaaggtg aggaactaaa ccatggccaa gttgaccagt
  9601 gccgttccgg tgctcaccgc gcgcgacgtc gccggagcgg tcgagttctg gaccgaccgg
  9661 ctcgggttct cccgggactt cgtggaggac gacttcgccg gtgtggtccg ggacgacgtg
  9721 accctgttca tcagcgcggt ccaggaccag gtggtgccgg acaacaccct ggcctgggtg
  9781 tgggtgcgcg gcctggacga gctgtacgcc gagtggtcgg aggtcgtgtc cacgaacttc
  9841 cgggacgcct ccgggccggc catgaccgag atcggcgagc agccgtgggg gcgggagttc
  9901 gccctgcgcg acccggccgg caactgcgtg cacttcgtgg ccgaggagca ggactgacac
  9961 gtgctacgag atttcgattc caccgccgcc ttctatgaaa ggttgggctt cggaatcgtt
10021 ttccgggacg ccggctggat gatcctccag cgcggggatc tcatgctgga gttcttcgcc
10081 caccccaact tgtttattgc agcttataat ggttacaaat aaagcaatag catcacaaat
10141 ttcacaaata aagcattttt ttcactgcat tctagttgtg gtttgtccaa actcatcaat
10201 gtatcttatc atgtctgtat accgtcgacc tctagctaga gcttggcgta atcatggtca
10261 tagctgtttc ctgtgtgaaa ttgttatccg ctcacaattc cacacaacat acgagccgga
10321 agcataaagt gtaaagcctg gggtgcctaa tgagtgagct aactcacatt aattgcgttg
10381 cgctcactgc ccgctttcca gtcgggaaac ctgtcgtgcc agctgcatta atgaatcggc
10441 caacgcgcgg ggagaggcgg tttgcgtatt gggcgctctt ccgcttcctc gctcactgac
10501 tcgctgcgct cggtcgttcg gctgcggcga gcggtatcag ctcactcaaa ggcggtaata
10561 cggttatcca cagaatcagg ggataacgca ggaaagaaca tgtgagcaaa aggccagcaa
10621 aaggccagga accgtaaaaa ggccgcgttg ctggcgtttt tccataggct ccgcccccct
10681 gacgagcatc acaaaaatcg acgctcaagt cagaggtggc gaaacccgac aggactataa
10741 agataccagg cgtttccccc tggaagctcc ctcgtgcgct ctcctgttcc gaccctgccg
10801 cttaccggat acctgtccgc ctttctccct tcgggaagcg tggcgctttc tcatagctca
10861 cgctgtaggt atctcagttc ggtgtaggtc gttcgctcca agctgggctg tgtgcacgaa
10921 ccccccgttc agcccgaccg ctgcgcctta tccggtaact atcgtcttga gtccaacccg
10981 gtaagacacg acttatcgcc actggcagca gccactggta acaggattag cagagcgagg
11041 tatgtaggcg gtgctacaga gttcttgaag tggtggccta actacggcta cactagaaga
11101 acagtatttg gtatctgcgc tctgctgaag ccagttacct tcggaaaaag agttggtagc
11161 tcttgatccg gcaaacaaac caccgctggt agcggtggtt tttttgtttg caagcagcag
11221 attacgcgca gaaaaaaagg atctcaagaa gatcctttga tcttttctac ggggtctgac
11281 gctcagtgga acgaaaactc acgttaaggg attttggtca tgagattatc aaaaaggatc
11341 ttcacctaga tccttttaaa ttaaaaatga agttttaaat caatctaaag tatatatgag
11401 taaacttggt ctgacagtta ccaatgctta atcagtgagg cacctatctc agcgatctgt
11461 ctatttcgtt catccatagt tgcctgactc cccgtcgtgt agataactac gatacgggag
11521 ggcttaccat ctggccccag tgctgcaatg ataccgcgag acccacgctc accggctcca
11581 gatttatcag caataaacca gccagccgga agggccgagc gcagaagtgg tcctgcaact
11641 ttatccgcct ccatccagtc tattaattgt tgccgggaag ctagagtaag tagttcgcca
11701 gttaatagtt tgcgcaacgt tgttgccatt gctacaggca tcgtggtgtc acgctcgtcg
11761 tttggtatgg cttcattcag ctccggttcc caacgatcaa ggcgagttac atgatccccc
11821 atgttgtgca aaaaagcggt tagctccttc ggtcctccga tcgttgtcag aagtaagttg
11881 gccgcagtgt tatcactcat ggttatggca gcactgcata attctcttac tgtcatgcca
11941 tccgtaagat gcttttctgt gactggtgag tactcaacca agtcattctg agaatagtgt
12001 atgcggcgac cgagttgctc ttgcccggcg tcaatacggg ataataccgc gccacatagc
12061 agaactttaa aagtgctcat cattggaaaa cgttcttcgg ggcgaaaact ctcaaggatc
12121 ttaccgctgt tgagatccag ttcgatgtaa cccactcgtg cacccaactg atcttcagca
12181 tcttttactt tcaccagcgt ttctgggtga gcaaaaacag gaaggcaaaa tgccgcaaaa
12241 aagggaataa gggcgacacg gaaatgttga atactcatac tcttcctttt tcaatattat
12301 tgaagcattt atcagggtta ttgtctcatg agcggataca tatttgaatg tatttagaaa
12361 aataaacaaa taggggttcc gcgcacattt ccccgaaaag tgccacctga c
```

In another aspect, the invention includes a vector comprising a first long terminal repeat (LTR) sequence, a U6 sequence, a direct repeat sequence of Cpf1, a first restriction site, a second restriction site, an EFS sequence, an antibiotic resistance sequence, a Woodchuck Hepatitis Virus 50 (WHP) Posttranscriptional Regulatory Element (WPRE) sequence, and a second LTR sequence (pLenti-U6-DR-crRNA-puro vector, Lenti-U6-crRNA for short). In certain embodiments, the first and/or second restriction site is a BsmBI restriction site. In one embodiment, the antibiotic resistance sequence is a puromycin resistance sequence. In one aspect, the vector comprises SEQ ID NO: 2 (FIGS. 18A-18B). In another aspect, the invention includes a vector optimized for primary cells (pSCO20_pLKO_U6-Cpf1crRNA-EFS-Thy11CO-sPA) (SEQ ID NO: 3) (FIGS. 19A-19EB).

```
pLenti-U6-DR-crRNA-puro vector (SEQ ID NO: 2):
      1 ttaatgtagt cttatgcaat actcttgtag tcttgcaaca tggtaacgat gagttagcaa
    61 catgccttac aaggagagaa aaagcaccgt gcatgccgat tggtggaagt aaggtggtac
  121 gatcgtgcct tattaggaag gcaacagacg ggtctgacat ggattggacg aaccactgaa
  181 ttgccgcatt gcagagatat tgtatttaag tgcctagctc gatacataaa cgggtctctc
  241 tggttagacc agatctgagc ctgggagctc tctggctaac tagggaaccc actgcttaag
  301 cctcaataaa gcttgccttg agtgcttcaa gtagtgtgtg cccgtctgtt gtgtgactct
  361 ggtaactaga gatccctcag acccttttag tcagtgtgga aaatctctag cagtggcgcc
  421 cgaacaggga cttgaaagcg aaagggaaac cagaggagct ctctcgacgc aggactcggc
  481 ttgctgaagc gcgcacggca agaggcgagg ggcggcgact ggtgagtacg ccaaaaattt
  541 tgactagcgg aggctagaag gagagagatg ggtgcgagag cgtcagtatt aagcggggga
  601 gaattagatc gcgatgggaa aaaattcggt taaggccagg gggaaagaaa aaatataaat
  661 taaaacatat agtatgggca agcagggagc tagaacgatt cgcagttaat cctggcctgt
  721 tagaaacatc agaaggctgt agacaaatac tgggacagct acaaccatcc cttcagacag
  781 gatcagaaga acttagatca ttatataata cagtagcaac cctctattgt gtgcatcaaa
  841 ggatagagat aaaagacacc aaggaagctt tagacaagat agaggaagag caaaacaaaa
  901 gtaagaccac cgcacagcaa gcggccgctg atcttcagac ctggaggagg agatatgagg
  961 gacaattgga gaagtgaatt atataaatat aaagtagtaa aaattgaacc attaggagta
1021 gcacccacca aggcaaagag aagagtggtg cagagagaaa aaagagcagt gggaatagga
1081 gctttgttcc ttgggttctt gggagcagca ggaagcacta tgggcgcagc gtcaatgacg
1141 ctgacggtac aggccagaca attattgtct ggtatagtgc agcagcagaa caatttgctg
1201 agggctattg aggcgcaaca gcatctgttg caactcacag tctggggcat caagcagctc
1261 caggcaagaa tcctggctgt ggaaagatac ctaaaggatc aacagctcct ggggatttgg
1321 ggttgctctg gaaaactcat ttgcaccact gctgtgcctt ggaatgctag ttggagtaat
1381 aaatctctgg aacagatttg gaatcacacg acctggatgg agtgggacag agaaattaac
1441 aattacacaa gcttaataca ctccttaatt gaagaatcgc aaaaccagca agaaaagaat
1501 gaacaagaat tattggaatt agataaatgg gcaagtttgt ggaattggtt taacataaca
1561 aattggctgt ggtatataaa attattcata atgatagtag gaggcttggt aggtttaaga
1621 atagtttttg ctgtactttc tatagtgaat agagttaggc agggatattc accattatcg
1681 tttcagaccc acctcccaac cccgagggga cccagagagg gcctatttcc catgattcct
1741 tcatatttgc atatacgata caaggctgtt agagagataa ttagaattaa tttgactgta
1801 aacacaaaga tattagtaca aaatacgtga cgtagaaagt aataatttct tgggtagttt
1861 gcagttttaa aattatgttt taaaatggac tatcatatgc ttaccgtaac ttgaaagtat
1921 ttcgatttct tggctttata tatcttGTGG AAAGGACGAA ACACCgTAAT TTCTACTAAG
1981 TGTAGATGAG ACGgaCGTCT Caagcttggc gtGGATCCGA TATCaactag atcttgagac
2041 aaatggcagt attcatccac aattttaaaa gaaaaggggg gattgggggg tacagtgcag
2101 gggaaagaat agtagacata atagcaacag acatacaaac taaagaatta caaaaacaaa
2161 ttacaaaaat tcaaaatttt cgggtttatt acagggacag cagagatcca ctttggcgcc
2221 ggctcgaggg ggcccgggga attcgctagc taggtcttga aaggagtggg aattggctcc
2281 ggtgcccgtc agtgggcaga gcgcacatcg cccacagtcc ccgagaagtt ggggggaggg
2341 gtcggcaatt gatccggtgc ctagagaagg tggcgcgggg taaactggga aagtgatgtc
2401 gtgtactggc tccgcctttt tcccgagggt gggggagaac cgtatataag tgcagtagtc
2461 gccgtgaacg ttctttttcg caacgggttt gccgccagaa cacaggaccg gttctagacg
2521 tacggccacc atgaccgagt acaagcccac ggtgcgcctc gccacccgcg acgacgtccc
2581 cagggccgta cgcaccctcg ccgccgcgtt cgccgactac cccgccacgc gccacaccgt
2641 cgatccggac cgccacatcg agcgggtcac cgagctgcaa gaactcttcc tcacgcgcgt
2701 cgggctcgac atcggcaagg tgtgggtcgc ggacgacggc gccgccgtgg cggtctggac
2761 cacgccggag agcgtcgaag cgggggcggt gttcgccgag atcggcccgc gcatggccga
2821 gttgagcggt tcccggctgg ccgcgcagca acagatggaa ggcctcctgg cgccgcaccg
2881 gcccaaggag cccgcgtggt tcctggccac cgtcggagtc tcgcccgacc accagggcaa
2941 gggtctgggc agcgccgtcg tgctccccgg agtggaggcg gccgagcgcg ccggggtgcc
3001 cgccttcctg gagacctccg cgccccgcaa cctccccttc tacgagcggc tcggcttcac
3061 cgtcaccgcc gacgtcgagg tgcccgaagg accgcgcacc tggtgcatga cccgcaagcc
3121 cggtgcctga acgcgttaag tcgacaatca acctctggat tacaaaattt gtgaaagatt
3181 gactggtatt cttaactatg ttgctccttt tacgctatgt ggatacgctg ctttaatgcc
3241 tttgtatcat gctattgctt cccgtatggc tttcattttc tcctccttgt ataaatcctg
3301 gttgctgtct ctttatgagg agttgtggcc cgttgtcagg caacgtggcg tggtgtgcac
3361 tgtgtttgct gacgcaaccc ccactggttg gggcattgcc accacctgtc agctcctttc
3421 cgggactttc gctttccccc tccctattgc cacggcggaa ctcatcgccg cctgccttgc
3481 ccgctgctgg acaggggctc ggctgttggg cactgacaat tccgtggtgt tgtcggggaa
3541 atcatcgtcc tttccttggc tgctcgcctg tgttgccacc tggattctgc gcgggacgtc
3601 cttctgctac gtcccttcgg ccctcaatcc agcggacctt ccttcccgcg gcctgctgcc
3661 ggctctgcgg cctcttccgc gtcttcgcct tcgccctcag acgagtcgga tctccctttg
3721 ggccgcctcc ccgcgtcgac tttaagacca atgacttaca aggcagctgt agatcttagc
3781 cactttttaa aagaaaaggg gggactggaa gggctaattc actcccaacg aagacaagat
3841 ctgctttttg cttgtactgg gtctctctgg ttagaccaga tctgagcctg ggagctctct
3901 ggctaactag ggaacccact gcttaagcct caataaagct tgccttgagt gcttcaagta
3961 gtgtgtgccc gtctgttgtg tgactctggt aactagagat ccctcagacc cttttagtca
4021 gtgtggaaaa tctctagcag tacgtatagt agttcatgtc atcttattat tcagtattta
4081 taacttgcaa agaaatgaat atcagagagt gagaggaact tgtttattgc agcttataat
4141 ggttacaaat aaagcaatag catcacaaat ttcacaaata aagcattttt ttcactgcat
4201 tctagttgtg gtttgtccaa actcatcaat gtatcttatc atgtctggct ctagctatcc
4261 cgcccctaac tccgcccatc ccgcccctaa ctccgcccag ttccgcccat tctccgcccc
4321 atggctgact aatttttttt atttatgcag aggccgaggc cgcctcggcc tctgagctat
4381 tccagaagta gtgaggaggc ttttttggag gcctagggac gtacccaatt cgccctatag
4441 tgagtcgtat tacgcgcgct cactggccgt cgttttacaa cgtcgtgact gggaaaaccc
4501 tggcgttacc caacttaatc gccttgcagc acatccccct ttcgccagct ggcgtaatag
4561 cgaagaggcc cgcaccgatc gcccttccca acagttgcgc agcctgaatg gcgaatggga
4621 cgcgccctgt agcggcgcat taagcgcggc gggtgtggtg gttacgcgca gcgtgaccgc
4681 tacacttgcc agcgccctag cgcccgctcc tttcgctttc ttcccttcct ttctcgccac
4741 gttcgccggc tttccccgtc aagctctaaa tcgggggctc cctttagggt tccgatttag
4801 tgctttacgg cacctcgacc ccaaaaaact tgattagggt gatggttcac gtagtgggcc
4861 atcgccctga tagacggttt ttcgcccttt gacgttggag tccacgttct ttaatagtgg
4921 actcttgttc caaactggaa caacactcaa ccctatctcg gtctattctt ttgatttata
4981 agggattttg ccgatttcgg cctattggtt aaaaaatgag ctgatttaac aaaaatttaa
5041 cgcgaatttt aacaaaatat taacgcttac aatttaggtg gcacttttcg gggaaatgtg
5101 cgcggaaccc ctatttgttt atttttctaa atacattcaa atatgtatcc gctcatgaga
5161 caataaccct gataaatgct tcaataatat tgaaaaagga agagtatgag tattcaacat
5221 ttccgtgtcg cccttattcc cttttttgcg gcattttgcc ttcctgtttt tgctcaccca
5281 gaaacgctgg tgaaagtaaa agatgctgaa gatcagttgg gtgcacgagt gggttacatc
5341 gaactggatc tcaacagcgg taagatcctt gagagttttc gccccgaaga acgttttcca
5401 atgatgagca cttttaaagt tctgctatgt ggcgcggtat tatcccgtat tgacgccggg
5461 caagagcaac tcggtcgccg catacactat tctcagaatg acttggttga gtactcacca
5521 gtcacagaaa agcatcttac ggatggcatg acagtaagag aattatgcag tgctgccata
5581 accatgagtg ataacactgc ggccaactta cttctgacaa cgatcggagg accgaaggag
5641 ctaaccgctt ttttgcacaa catgggggat catgtaactc gccttgatcg ttgggaaccg
5701 gagctgaatg aagccatacc aaacgacgag cgtgacacca cgatgcctgt agcaatggca
5761 acaacgttgc gcaaactatt aactggcgaa ctacttactc tagcttcccg gcaacaatta
5821 atagactgga tggaggcgga taaagttgca ggaccacttc tgcgctcggc ccttccggct
5881 ggctggttta ttgctgataa atctggagcc ggtgagcgtg ggtctcgcgg tatcattgca
5941 gcactggggc cagatggtaa gccctcccgt atcgtagtta tctacacgac ggggagtcag
6001 gcaactatgg atgaacgaaa tagacagatc gctgagatag gtgcctcact gattaagcat
6061 tggtaactgt cagaccaagt ttactcatat atactttaga ttgatttaaa acttcatttt
6121 taatttaaaa ggatctaggt gaagatcctt tttgataatc tcatgaccaa aatcccttaa
6181 cgtgagtttt cgttccactg agcgtcagac cccgtagaaa agatcaaagg atcttcttga
6241 gatccttttt ttctgcgcgt aatctgctgc ttgcaaacaa aaaaaccacc gctaccagcg
6301 gtggtttgtt tgccggatca agagctacca actctttttc cgaaggtaac tggcttcagc
6361 agagcgcaga taccaaatac tgttcttcta gtgtagccgt agttaggcca ccacttcaag
6421 aactctgtag caccgcctac atacctcgct ctgctaatcc tgttaccagt ggctgctgcc
6481 agtggcgata agtcgtgtct taccgggttg gactcaagac gatagttacc ggataaggcg
6541 cagcggtcgg gctgaacggg gggttcgtgc acacagccca gcttggagcg aacgacctac
6601 accgaactga gatacctaca gcgtgagcta tgagaaagcg ccacgcttcc cgaagggaga
6661 aaggcggaca ggtatccggt aagcggcagg gtcggaacag gagagcgcac gagggagctt
6721 ccagggggaa acgcctggta tctttatagt cctgtcgggt ttcgccacct ctgacttgag
6781 cgtcgatttt tgtgatgctc gtcagggggg cggagcctat ggaaaaacgc cagcaacgcg
6841 gcctttttac ggttcctggc cttttgctgg ccttttgctc acatgttctt tcctgcgtta
6901 tcccctgatt ctgtggataa ccgtattacc gcctttgagt gagctgatac cgctcgccgc
6961 agccgaacga ccgagcgcag cgagtcagtg agcgaggaag cggaagagcg cccaatacgc
7021 aaaccgcctc tccccgcgcg ttggccgatt cattaatgca gctggcacga caggtttccc
7081 gactggaaag cgggcagtga gcgcaacgca attaatgtga gttagctcac tcattaggca
7141 ccccaggctt tacactttat gcttccggct cgtatgttgt gtggaattgt gagcggataa
7201 caatttcaca caggaaacag ctatgaccat gattacgcca agcgcgcaat taaccctcac
7261 taaagggaac aaaagctgga gctgcaagc
pSC020_pLKO_U6-Cpf1crRNA-EFS-Thy11CO-sPA vector (SEQ ID NO: 3)
      1 ttaatgtagt cttatgcaat actcttgtag tcttgcaaca tggtaacgat gagttagcaa
    61 catgccttac aaggagagaa aaagcaccgt gcatgccgat tggtggaagt aaggtggtac
  121 gatcgtgcct tattaggaag gcaacagacg ggtctgacat ggattggacg aaccactgaa
  181 ttgccgcatt gcagagatat tgtatttaag tgcctagctc gatacataaa cgggtctctc
  241 tggttagacc agatctgagc ctgggagctc tctggctaac tagggaaccc actgcttaag
  301 cctcaataaa gcttgccttg agtgcttcaa gtagtgtgtg cccgtctgtt gtgtgactct
  361 ggtaactaga gatccctcag acccttttag tcagtgtgga aaatctctag cagtggcgcc
  421 cgaacaggga cttgaaagcg aaagggaaac cagaggagct ctctcgacgc aggactcggc
  481 ttgctgaagc gcgcacggca agaggcgagg ggcggcgact ggtgagtacg ccaaaaattt
  541 tgactagcgg aggctagaag gagagagatg ggtgcgagag cgtcagtatt aagcggggga
  601 gaattagatc gcgatgggaa aaaattcggt taaggccagg gggaaagaaa aaatataaat
  661 taaaacatat agtatgggca agcagggagc tagaacgatt cgcagttaat cctggcctgt
  721 tagaaacatc agaaggctgt agacaaatac tgggacagct acaaccatcc cttcagacag
  781 gatcagaaga acttagatca ttatataata cagtagcaac cctctattgt gtgcatcaaa
  841 ggatagagat aaaagacacc aaggaagctt tagacaagat agaggaagag caaaacaaaa
  901 gtaagaccac cgcacagcaa gcggccgctg atcttcagac ctggaggagg agatatgagg
  961 gacaattgga gaagtgaatt atataaatat aaagtagtaa aaattgaacc attaggagta
1021 gcacccacca aggcaaagag aagagtggtg cagagagaaa aaagagcagt gggaatagga
1081 gctttgttcc ttgggttctt gggagcagca ggaagcacta tgggcgcagc gtcaatgacg
1141 ctgacggtac aggccagaca attattgtct ggtatagtgc agcagcagaa caatttgctg
1201 agggctattg aggcgcaaca gcatctgttg caactcacag tctggggcat caagcagctc
1261 caggcaagaa tcctggctgt ggaaagatac ctaaaggatc aacagctcct ggggatttgg
1321 ggttgctctg gaaaactcat ttgcaccact gctgtgcctt ggaatgctag ttggagtaat
1381 aaatctctgg aacagatttg gaatcacacg acctggatgg agtgggacag agaaattaac
1441 aattacacaa gcttaataca ctccttaatt gaagaatcgc aaaaccagca agaaaagaat
1501 gaacaagaat tattggaatt agataaatgg gcaagtttgt ggaattggtt taacataaca
1561 aattggctgt ggtatataaa attattcata atgatagtag gaggcttggt aggtttaaga
1621 atagtttttg ctgtactttc tatagtgaat agagttaggc agggatattc accattatcg
1681 tttcagaccc acctcccaac cccgagggga cccagagagg gcctatttcc catgattcct
1741 tcatatttgc atatacgata caaggctgtt agagagataa ttagaattaa tttgactgta
1801 aacacaaaga tattagtaca aaatacgtga cgtagaaagt aataatttct tgggtagttt
1861 gcagttttaa aattatgttt taaaatggac tatcatatgc ttaccgtaac ttgaaagtat
1921 ttcgatttct tggctttata tatcttGTGG AAAGGACGAA ACACCgTAAT TTCTACTAAG
1981 TGTAGATGAG ACGgaCGTCT Caagcttggc gtGGATCCGA TATCaactag atcttgagac
2041 aaatggcagt attcatccac aattttaaaa gaaaaggggg gattgggggg tacagtgcag
2101 gggaaagaat agtagacata atagcaacag acatacaaac taaagaatta caaaaacaaa
2161 ttacaaaaat tcaaaatttt cgggtttatt acagggacag cagagatcca ctttggcgcc
2221 ggctcgaggg ggcccgggga attcgctagc taggtcttga aaggagtggg aattggctcc
2281 ggtgcccgtc agtgggcaga gcgcacatcg cccacagtcc ccgagaagtt ggggggaggg
2341 gtcggcaatt gatccggtgc ctagagaagg tggcgcgggg taaactggga aagtgatgtc
2401 gtgtactggc tccgcctttt tcccgagggt gggggagaac cgtatataag tgcagtagtc
2461 gccgtgaacg ttctttttcg caacgggttt gccgccagaa cacaggaccg gttctagacg
2521 tacggccacc ATGAACCCAG CCATCAGCGT CGCTCTCCTG CTCTCAGTCT TGCAGGTGTC
2581 CCGAGGGCAG AAGGTGACCA GCCTGACAGC CTGCCTGGTG AACCAAAACC TTCGCCTGGA
2641 CTGCCGCCAT GAGAATAACA CCAAGGATAA CTCCATCCAG CATGAGTTCA GCCTGACCCG
2701 AGAGAAGAGG AAGCACGTGC TCTCAGGCAC CCTTGGGATA CCCGAGCACA CGTACCGCTC
2761 CCGCGTCACC CTCTCCAACC AGCCCTATAT CAAGGTCCTT ACCCTAGCCA ACTTCACCAC
2821 CAAGGATGAG GGCGACTACT TTTGTGAGCT TCGCGTAAGT GGCGCGAATC CCATGAGCTC
2881 CAATAAAAGT ATCAGTGTGT ATAGAGACAA GCTGGTCAAG TGTGGCGGCA TAAGCCTGCT
2941 GGTTCAGAAC ACATCCTGGA TGCTGCTGCT GCTGCTTTCC CTCTCCCTCC TCCAAGCCCT
3001 GGACTTCATT TCTCTGTGAa gcgctAATAA AAGATCTTTA TTTTCATTAG ATCTGTGTGT
3061 TGGTTTTTTG TGTGacgtgc ggtcgacttt aagaccaatg acttacaagg cagctgtaga
3121 tcttagccac tttttaaaag aaaagggggg actggaaggg ctaattcact cccaacgaag
3181 acaagatctg ctttttgctt gtactgggtc tctctggtta gaccagatct gagcctggga
3241 gctctctggc taactaggga acccactgct taagcctcaa taaagcttgc cttgagtgct
3301 tcaagtagtg tgtgcccgtc tgttgtgtga ctctggtaac tagagatccc tcagaccctt
3361 ttagtcagtg tggaaaatct ctagcagtac gtatagtagt tcatgtcatc ttattattca
3421 gtatttataa cttgcaaaga aatgaatatc agagagtgag aggaacttgt ttattgcagc
3481 ttataatggt tacaaataaa gcaatagcat cacaaatttc acaaataaag catttttttc
3541 actgcattct agttgtggtt tgtccaaact catcaatgta tcttatcatg tctggctcta
3601 gctatcccgc ccctaactcc gcccatcccg cccctaactc cgcccagttc cgcccattct
3661 ccgccccatg gctgactaat tttttttatt tatgcagagg ccgaggccgc ctcggcctct
3721 gagctattcc agaagtagtg aggaggcttt tttggaggcc tagggacgta cccaattcgc
3781 cctatagtga gtcgtattac gcgcgctcac tggccgtcgt tttacaacgt cgtgactggg
3841 aaaaccctgg cgttacccaa cttaatcgcc ttgcagcaca tccccctttc gccagctggc
3901 gtaatagcga agaggcccgc accgatcgcc cttcccaaca gttgcgcagc ctgaatggcg
3961 aatgggacgc gccctgtagc ggcgcattaa gcgcggcggg tgtggtggtt acgcgcagcg
4021 tgaccgctac acttgccagc gccctagcgc ccgctccttt cgctttcttc ccttcctttc
4081 tcgccacgtt cgccggcttt ccccgtcaag ctctaaatcg ggggctccct ttagggttcc
4141 gatttagtgc tttacggcac ctcgacccca aaaaacttga ttagggtgat ggttcacgta
4201 gtgggccatc gccctgatag acggtttttc gccctttgac gttggagtcc acgttcttta
4261 atagtggact cttgttccaa actggaacaa cactcaaccc tatctcggtc tattcttttg
4321 atttataagg gattttgccg atttcggcct attggttaaa aaatgagctg atttaacaaa
4381 aatttaacgc gaattttaac aaaatattaa cgcttacaat ttaggtggca cttttcgggg
4441 aaatgtgcgc ggaaccccta tttgtttatt tttctaaata cattcaaata tgtatccgct
4501 catgagacaa taaccctgat aaatgcttca ataatattga aaaaggaaga gtatgagtat
4561 tcaacatttc cgtgtcgccc ttattccctt ttttgcggca ttttgccttc ctgtttttgc
4621 tcacccagaa acgctggtga aagtaaaaga tgctgaagat cagttgggtg cacgagtggg
4681 ttacatcgaa ctggatctca acagcggtaa gatccttgag agttttcgcc ccgaagaacg
4741 ttttccaatg atgagcactt ttaaagttct gctatgtggc gcggtattat cccgtattga
4801 cgccgggcaa gagcaactcg gtcgccgcat acactattct cagaatgact tggttgagta
4861 ctcaccagtc acagaaaagc atcttacgga tggcatgaca gtaagagaat tatgcagtgc
4921 tgccataacc atgagtgata acactgcggc caacttactt ctgacaacga tcggaggacc
4981 gaaggagcta accgcttttt tgcacaacat gggggatcat gtaactcgcc ttgatcgttg
5041 ggaaccggag ctgaatgaag ccataccaaa cgacgagcgt gacaccacga tgcctgtagc
5101 aatggcaaca acgttgcgca aactattaac tggcgaacta cttactctag cttcccggca
5161 acaattaata gactggatgg aggcggataa agttgcagga ccacttctgc gctcggccct
5221 tccggctggc tggtttattg ctgataaatc tggagccggt gagcgtgggt ctcgcggtat
5281 cattgcagca ctggggccag atggtaagcc ctcccgtatc gtagttatct acacgacggg
5341 gagtcaggca actatggatg aacgaaatag acagatcgct gagataggtg cctcactgat
5401 taagcattgg taactgtcag accaagttta ctcatatata ctttagattg atttaaaact
5461 tcatttttaa tttaaaagga tctaggtgaa gatccttttt gataatctca tgaccaaaat
5521 cccttaacgt gagttttcgt tccactgagc gtcagacccc gtagaaaaga tcaaaggatc
5581 ttcttgagat cctttttttc tgcgcgtaat ctgctgcttg caaacaaaaa aaccaccgct
5641 accagcggtg gtttgtttgc cggatcaaga gctaccaact ctttttccga aggtaactgg
5701 cttcagcaga gcgcagatac caaatactgt tcttctagtg tagccgtagt taggccacca
5761 cttcaagaac tctgtagcac cgcctacata cctcgctctg ctaatcctgt taccagtggc
5821 tgctgccagt ggcgataagt cgtgtcttac cgggttggac tcaagacgat agttaccgga
5881 taaggcgcag cggtcgggct gaacgggggg ttcgtgcaca cagcccagct tggagcgaac
5941 gacctacacc gaactgagat acctacagcg tgagctatga gaaagcgcca cgcttcccga
6001 agggagaaag gcggacaggt atccggtaag cggcagggtc ggaacaggag agcgcacgag
6061 ggagcttcca gggggaaacg cctggtatct ttatagtcct gtcgggtttc gccacctctg
6121 acttgagcgt cgatttttgt gatgctcgtc aggggggcgg agcctatgga aaaacgccag
6181 caacgcggcc tttttacggt tcctggcctt ttgctggcct tttgctcaca tgttctttcc
6241 tgcgttatcc cctgattctg tggataaccg tattaccgcc tttgagtgag ctgataccgc
6301 tcgccgcagc cgaacgaccg agcgcagcga gtcagtgagc gaggaagcgg aagagcgccc
6361 aatacgcaaa ccgcctctcc ccgcgcgttg gccgattcat taatgcagct ggcacgacag
6421 gtttcccgac tggaaagcgg gcagtgagcg caacgcaatt aatgtgagtt agctcactca
6481 ttaggcaccc caggctttac actttatgct tccggctcgt atgttgtgtg gaattgtgag
6541 cggataacaa tttcacacag gaaacagcta tgaccatgat tacgccaagc gcgcaattaa
6601 ccctcactaa agggaacaaa agctggagct gcaagc
```

In another aspect, the invention includes a crRNA array comprising a 5′ nucleotide sequence that is homologous to a first nucleotide sequence on a vector, a first crRNA sequence, a direct repeat sequence of Cpf1, a second crRNA sequence, a terminator sequence, and a 3′ sequence that is homologous to a second sequence on the vector. In one embodiment, the terminator sequence is a U6 terminator sequence. The vector can include any vector known in the art or described herein. In certain embodiments the vector comprises the pLenti-U6-DR-crRNA-puro vector. The crRNA sequences can be designed to target any gene of interest or nucleotide sequence of interest.

In yet another aspect, the invention includes a double knockout crRNA expression vector (pLenti-U6-DR-cr1-DR-cr2-puro). The vector comprises a first LTR sequence, a promoter sequence, a first direct repeat sequence of Cpf1, a first crRNA sequence, a second direct repeat sequence of Cpf1, a second crRNA sequence, a terminator sequence, an EFS sequence, a WPRE sequence, and a second LTR sequence. In one embodiment, the promoter sequence is a U6 promoter sequence. In one embodiment, the terminator sequence is a U6 terminator sequence.

The crRNA sequences can target any gene or nucleotide sequence of interest. In certain embodiments, the first crRNA sequence is complementary to a gene selected from the group consisting of Pten and Nf1, and the second crRNA sequence is complementary to a gene selected from the group consisting of Pten and Nf1. The first and second crRNAs can target the same gene/sequence or different genes/sequences. The vector can further comprise additional crRNA sequences totaling up to 3, 4, 5, 6, 7, 8, 9, 10, 11, 12, 13, 14, 15, 16, 17, 18, 19 or 20 crRNAs in one vector.

In one aspect, the invention includes a Cpf1 crRNA array screening (CCAS) library. In another aspect, the invention includes a Massively-Parallel crRNA Array Profiling (MCAP) library. In certain embodiments, the library comprises a plurality of the crRNA arrays of the invention cloned into a plurality of the vectors of the invention. In certain embodiments, the MCAP library comprises a plurality of crRNA arrays targeting pairwise combinations of genes significantly mutated in human metastases. In certain embodiments, the crRNA arrays in the library comprise at least one nucleotide sequence selected from the group consisting of SEQ ID NOs. 4-9,708. In certain embodiments, the crRNA arrays in the library consist of the nucleotide sequences of SEQ ID NOs. 4-9,708. In certain embodiments, the crRNA arrays in the library comprise at least one nucleotide sequence selected from the group consisting of SEQ ID NOs. 9,762-21,695. In certain embodiments, the crRNA arrays in the library consist of the nucleotide sequences of SEQ ID NOs. 9,762-21,695.

The invention also provides, in one aspect, a kit comprising a CCAS library comprising a plurality of vectors comprising a plurality of crRNA arrays, wherein the crRNA arrays comprise a nucleotide sequence selected from the group consisting of SEQ ID NOs. 4-9,708. In another aspect, the invention includes a kit comprising a MCAP library comprising a plurality of vectors comprising a plurality of crRNA arrays, wherein the crRNA arrays comprise a nucleotide sequence selected from the group consisting of SEQ ID NOs. 9,762-21,695. Also included in the kits are instructional materials for use thereof. Instructional material can include directions for using the components of the kit as well as instructions and guidance for interpreting the results. In one aspect, the kit comprises at least one additional crRNA sequence that is complementary to at least one additional target sequence. For example, the kit is capable of multiplexing 3 or more crRNAs in each array in order to study triple knockouts and even higher-dimension (i.e., quadruple or higher) genetic interactions.

Methods Described herein are multiplexed Cpf1 screens that provide a powerful tool for studying genetic interactions with unparalleled simplicity and specificity. The Cpf1 crRNA array screening (CCAS) and MCAP (Massively-parallel crRNA array profiling) technologies enable rapid identification of all combinations of double inhibition of two targets simultaneously. The methods described herein can be broadly applied to many cell types of interest, including but not limited to cancer cells. As shown in the present study (FIGS. 1A-20F and 26A-34), CCAS and MCAP can be used in mammalian cells for high-throughput, high-dimensional screening. A set of highly quantitative algorithms was developed, and this was used to generate unbiased profiles of genetic interactions in tumor suppression and metastasis, which were dismantled upon Cpf1-mediated double-mutagenesis. Particularly, in a more complex biological process such as the multi-step metastatic process, the screen was capable of detecting robust signatures of selection and revealing modes and patterns of clonal expansion of complex pools of double mutants in vivo. Technology-wise, establishment of Cpf1 crRNA array libraries, readout and mapping platform, as well as customized computational pipelines, enables more comprehensive combinatorial screens through a single crRNA array. This technology is readily extendable to multiplexing 3 or more crRNAs in each array in order to study triple knockouts and even higher-dimension genetic interactions. Triple-, quadruple- or higher dimensional screens are easily feasible with Cpf1 crRNA array screening system, which were exponentially challenging for methods depending on Cas9. The extremely simplified library construction enables direct double knockout at greatly reduced cost and effort. Particularly in an in vivo setting, simplicity directly empowers feasibility.

The methods can also encompass additional applications in immune cells for immunotherapy screening and enhancement. Editing of primary immune cells (such as Dendritic cells (DCs)) was demonstrated herein (FIG. 16). This allows direct application of CCAS technology to screen for combinatorial factors that modulate immunotherapy and engineering immune cells with desired or improved functions.

Applications in primary cells for improving regenerative medicine are also encompassed by this approach. Editing of freshly isolated primary cells (such as Endothelial cells (ECs)) was demonstrated herein (FIG. 16). This allows direct application of the CCAS technology to screen for combinatorial factors that modulate regenerative medicine.

In one aspect, the invention includes a method for simultaneously mutagenizing multiple target sequences in a cell. The method comprises administering to the cell a CCAS library. The CCAS library comprises a plurality of vectors comprising a plurality of crRNA arrays. The crRNA arrays comprise a 5′ nucleotide sequence that is homologous to a first nucleotide sequence on the vector, a first crRNA sequence, a direct repeat sequence of Cpf1, a second crRNA sequence, a terminator sequence, and a 3′ sequence that is homologous to a second sequence on the vector, and wherein the first crRNA is complementary to a first target sequence and the second crRNA is complementary to a second target sequence. In certain embodiments, the plurality of crRNA arrays in the CCAS library comprises a nucleotide sequence selected from the group consisting of SEQ ID NOs. 4-9,708. The method can also include additional crRNA sequences complementary to additional target sequences. For example, additional crRNA sequences totaling up to 3, 4, 5, 6, 7, 8, 9, 10, 11, 12, 13, 14, 15, 16, 17, 18, 19 or 20 crRNAs can be included in the methods as described herein.

In another aspect, the invention includes a method for simultaneously mutagenizing multiple target sequences in a cell comprising administering to the cell a MCAP library. The MCAP library comprises a plurality of vectors comprising a plurality of crRNA arrays. The crRNA arrays comprise a 5′ nucleotide sequence that is homologous to a first nucleotide sequence on the vector, a first crRNA sequence, a direct repeat sequence of Cpf1, a second crRNA sequence, a terminator sequence, and a 3′ sequence that is homologous to a second sequence on the vector, and wherein the first crRNA is complementary to a first target sequence and the second crRNA is complementary to a second target sequence. In certain embodiments, the plurality of crRNA arrays in the MCAP library comprises a nucleotide sequence selected from the group consisting of SEQ ID NOs. 9,762-21,695.

By ‘target sequence’ is meant any nucleic acid sequence or gene of interest targeted to be mutated by the methods described herein.

Any type of cell can be mutagenized by the methods described herein, including but not limited to cancer cells, immune cells, cell lines, hybridomas, primary cells, T cells, dendritic cells (DCs), endothelial cells, brain endothelial cells, macrophages, monocytes, CD8+ cells, CD4+ cells, T regulatory (Treg) cells, B cells, Natural Killer cells (NKs), and stem cells.

Another aspect of the invention includes a method of identifying synergistic drivers of transformation and/or tumorigenesis and/or metastasis in vivo. The method comprises administering to an animal cells mutagenized by a CCAS library. The CCAS library comprises a plurality of vectors comprising a plurality of crRNA arrays. Each crRNA array comprises a 5′ nucleotide sequence that is homologous to a first nucleotide sequence on the vector, a first crRNA sequence, a direct repeat sequence of Cpf1, a second crRNA sequence, a terminator sequence, and a 3′ sequence that is homologous to a second sequence on the vector. The first crRNA is complementary to a first target sequence and the second crRNA is complementary to a second target sequence. A nucleotide from a tumor from the animal are sequenced, and the data are analyzed to identify the synergistic drivers of transformation and/or tumorigenesis.

Still another aspect of the invention includes a method of identifying synergistic drivers of transformation and/or tumorigenesis and/or metastasis in vivo comprising administering cells mutagenized by a MCAP library to an animal. The MCAP library comprises a plurality of vectors comprising a plurality of crRNA arrays. Each crRNA array comprises a 5′ nucleotide sequence that is homologous to a first nucleotide sequence on the vector, a first crRNA sequence, a direct repeat sequence of Cpf1, a second crRNA sequence, a terminator sequence, and a 3′ sequence that is homologous to a second sequence on the vector. In certain embodiments, the MCAP library comprises a plurality of crRNA arrays targeting pairwise combinations of genes significantly mutated in human metastases. The first crRNA is complementary to a first target sequence and the second crRNA is complementary to a second target sequence. A nucleotide from a tumor from the animal are sequenced, and the data are analyzed to identify the synergistic drivers of transformation and/or tumorigenesis.

Yet another aspect of the invention includes an in vivo method for identifying and mapping genetic interactions. The method comprises administering cells mutagenized by a CCAS library to an animal. The CCAS library comprises a plurality of vectors comprising a plurality of crRNA arrays. Each crRNA array comprises a 5′ nucleotide sequence that is homologous to a first nucleotide sequence on the vector, a first crRNA sequence, a direct repeat sequence of Cpf1, a second crRNA sequence, a U6 terminator sequence, and a 3′ sequence that is homologous to a second sequence on the vector. The first crRNA is complementary to a first target sequence, and the second crRNA is complementary to a second target sequence. A nucleotide from a tumor and/or tissue and/or cell of the animal are sequenced, and the data are analyzed to identify and map the genetic interactions.

Another aspect of the invention includes an in vivo method for identifying and mapping genetic interactions. The method comprises administering to an animal cells mutagenized by a MCAP library. The MCAP library comprises a plurality of vectors comprising a plurality of crRNA arrays. Each crRNA array comprises a 5′ nucleotide sequence that is homologous to a first nucleotide sequence on the vector, a first crRNA sequence, a direct repeat sequence of Cpf1, a second crRNA sequence, a terminator sequence, and a 3′ sequence that is homologous to a second sequence on the vector. The first crRNA is complementary to a first target sequence, and the second crRNA is complementary to a second target sequence. A nucleotide (DNA or RNA) from a tumor and/or tissue and/or cell of the animal are sequenced, and the data are analyzed to identify and map the genetic interactions.

In certain embodiments of the methods, the plurality of crRNA arrays comprises SEQ ID NOs. 4-9,708. In certain embodiments of the methods, the plurality of crRNA arrays comprises SEQ ID NOs. 9,762-21,695. In certain embodiments, the methods further comprise wherein the crRNA comprises additional crRNA sequences that are complementary to additional target sequences. The methods of the invention are capable of multiplexing 3 or more crRNAs in each array in order to study triple knockouts and even higher-dimension genetic interactions.

Nucleotide sequencing or “sequencing”, as it is commonly known in the art, can be performed by standard methods commonly known to one of ordinary skill in the art. In certain embodiments of the invention, sequencing is performed by targeted capture sequencing.

Targeted captured sequencing can be performed as described herein, or by methods commonly performed by one of ordinary skill in the art. In certain embodiments of the invention, sequencing is performed via next-generation sequencing. Next-generation sequencing (NGS), also known as high-throughput sequencing, is used herein to describe a number of different modern sequencing technologies that allow to sequence DNA and RNA much more quickly and cheaply than the previously used Sanger sequencing (Metzker, 2010, Nature Reviews Genetics 11.1: 31-46). It is based on micro- and nanotechnologies to reduce the size of sample, the reagent costs, and to enable massively parallel sequencing reactions. It can be highly multiplexed, which allows simultaneous sequencing and analysis of millions of samples. NGS includes first, second, third as well as subsequent Next Generations Sequencing technologies. Data generated from NGS can be analyzed via a broad range of computational tools and statistical methods including but not limited to those described herein. Sequencing can also be performed at the single cell level, e.g. single cell sequencing. Sequencing can be performed on DNA as well as RNA (e.g. RNASeq). The wide variety of analysis can be appreciated and performed by those skilled in the art.

Mutagenizing a cell can include introducing mutations throughout the genome of the cell. The mutations introduced can be any combination of insertions or deletions, including but not limited to a single base insertion, a single base deletion, a frameshift, a rearrangement, and an insertion or deletion of 2, 3, 4, 5, 6, 7, 8, 9, 10, 15, 20, 25, 30, 40, 50, 60, 70, 80, 90, 100, 150, 200, 250, 300, any and all numbers in between, bases. The mutation can occur in a gene or in a non-coding region.

In certain embodiments of the invention, the animal is a mouse. Other animals that can be used include but are not limited to rats, rabbits, dogs, cats, horses, pigs, cows and birds. In certain embodiments, the animal is a human. The sgRNA library can be administered to an animal by any means standard in the art. For example the vectors can be injected into the animal. The injections can be intravenous, subcutaneous, intraperitoneal, or directly into a tissue or organ. In certain embodiments, the sgRNA library is adoptively transferred to the animal.

Cpf1-Flip In certain aspects, the invention includes compositions and methods for sequential mutagnesis in a cell using the Cpf1-Flip system.

In a large variety of biological and pathological processes, genetic mutations or alterations are often acquired in a sequential manner. In evolution and speciation, the genomes of organisms acquire mutations constantly and are subjected to natural selection. In genetically complex disorders such as cancer, multi-step mutagenesis is often a major obstacle for effective treatments. Cancers evolve through an ongoing process of mutation-selection balance, where initial mutations are selected for, or against, in vivo, followed by subsequent acquisition of additional mutations as the tumor grows. Since the initial set of oncogenic “driver” mutations is generally what starts and sustains tumor growth, targeted molecular therapies are often chosen to specifically attack such oncogenic dependencies. However, the selection pressures of treatment favor secondary mutations that confer drug resistance, leading to relapse. Thus, the process of cancer evolution by sequential mutagenesis stymies these therapies via continuous diversification and adaptation to the tumor microenvironment, eventually exhausting available treatment options. Even with the advent of cancer immunotherapy, where checkpoint blockade is increasingly being utilized in the clinic, the acquisition of secondary mutations that abolish T cell receptor (TCR)—antigen—major histocompatibility complex (MHC) recognition can still lead to immune escape and ultimately negate the effect of immunotherapy. Thus, the ability to perform sequential and precise mutagenesis is critical for studying biological processes with multi-stage genetic events such as development and evolution, as well as the pathogenesis of complex diseases such as cancer.

From a genetic engineering perspective, stepwise mutagenesis or perturbation is a powerful technique for precise genetic manipulation of cells and live organisms. Multiple methods have been employed to achieve this end. In the pre-recombinant DNA era, stepwise perturbation was often done by multiple rounds of random mutagenesis using chemical or physical carcinogens followed by artificial selection. The subsequent discovery and application of recombinase systems such as Cre-loxP, Flp-FRT and cpC31-att enabled inducible genetic events. In these systems, the DNA recombinase (i.e. Cre) specifically recognizes its target DNA sequence motif (i.e. loxP) and catalyzes recombination between two such target sites. Depending on the configuration of the target sites, targeted recombinases can be utilized for DNA excision, translocation, and/or inversion. However, the floxed genomic loci underlying Cre-based systems must be pre-engineered on a gene-by-gene basis. This process of generating new floxed alleles for each unique application is time and labor intensive, further limiting the feasibility of multiplexed Cre recombination.

More recently, precisely targeted and customizable mutagenesis was simplified by the discovery of RNA-guided endonucleases (RGNs) Cas9 and Cpf1. RGNs can induce double strand DNA breaks, subsequently generating insertions and deletions at the target site. This process is precisely targeted based on the sequences of CRISPR RNAs (crRNAs), which complex with RGNs to enable and guide their nuclease functions. Unlike with Cre recombination, CRISPR crRNAs can be easily transferred to target cells through transfection or viral vectors, thus obviating the need to pre-engineer the host genome for each target gene. In contrast to Cas9, the most widely utilized RGN to date, Cpf1 is a single component RGN that does not depend on trans-activating RNA and can autonomously process CRISPR-RNA (crRNA) arrays. These features have made Cpf1 particularly attractive for multiplexed mutagenesis. In addition to several studies in mammalian systems, Cpf1-mediated mutagenesis and transcriptional repression have now been successfully applied in plants. Furthermore, chemical modifications on Cpf1 mRNA and crRNAs have been identified that can improve cutting efficiency. Cpf1 can also process crRNAs from mRNAs expressed by a Pol II promoter, further enabling flexible transcriptional control.

Sequential mutagenesis using Cas9 has been demonstrated in ex vivo organoid cultures. However, this approach required sequentially introducing each sgRNA in culture, one at a time, limiting its broader applicability. In particular, the sequential introduction of different sgRNAs would be impractical for library-scale screening or any in vivo experimental designs. Prior to this disclosure, conditional sequential mutagenesis using RGNs has not yet been demonstrated.

Herein, a flexible sequential mutagenesis system was created through inducible inversion of a single crRNA array (Cpf1-Flip) and its simplicity demonstrated in stepwise multiplexed gene editing in mammalian cells for modeling sequential genetic events, such as in cancer. Cpf1-Flip was further applied to model the acquisition of resistance mutations to immunotherapy in a pooled mutagenesis setting, demonstrating the feasibility of Cpf1-Flip for conducting sequential genetic studies. This system can be utilized for multi-step mutagenesis of any genes in the genome for interrogating complex genetic events with temporal control.

In certain aspects, the invention includes a crRNA Flip Array. In one embodiment, the crRNA FlipArray comprises a first crRNA sequence, 4-10 consecutive thymidines, a second inverted crRNA sequence, 4-10 consecutive adenines, and a third inverted direct repeat sequence. In one embodiment, the first crRNA sequence comprises six consecutive thymidines. In one embodiment, the second inverted crRNA sequence comprises six consecutive adenines. The crRNA Flip Array can be included in any vector known to one of ordinary skill in the art.

In one embodiment, the invention includes a vector comprising a first promoter, a Cpf1 sequence, a second promoter, a first Cpf1 direct repeat sequence, a lox66 sequence, a second Cpf1 direct repeat sequence, two inverted restriction sites, an inverted lox71 sequence, and a crRNA FlipArray, wherein the crRNA FlipArray comprises a first crRNA sequence, 4-10 consecutive thymidines, a second inverted crRNA sequence, 4-10 consecutive adenines, and a third inverted direct repeat sequence.

In certain embodiments, the vector comprises SEQ ID NO: 21,697. In one embodiment, the first promoter is an EFS promoter. In one embodiment, the EFS promoter drives expression of Cpf1. In one embodiment, the second promoter is a U6 promoter. In one embodiment, the U6 promoter drives expression of the crRNA FlipArray. In one embodiment, the first promoter and the second promoter are in opposite orientations. In one embodiment, the vector further comprises an antibiotic resistance marker. In one embodiment, the antibiotic resistance marker is a puromycin resistance sequence. In one embodiment, the restriction sites are BsmbI restriction sites. In one embodiment, the Cpf1 sequence is a Lachnospiraceae bacterium Cpf1 (LbCpf1) sequence. In one embodiment, any one of the first, second, or third, direct repeat sequences is from LbCpf1.

In one aspect, the invention includes a gene editing system capable of inducible, sequential mutagenesis in a cell. The system comprising a vector and a Cre recombinase, wherein the vector comprising a first promoter, a Cpf1 sequence, a second promoter, a first Cpf1 direct repeat sequence, a lox66 sequence, a second Cpf1 direct repeat sequence, two inverted restriction sites, an inverted lox71 sequence, and a crRNA FlipArray, wherein the crRNA FlipArray comprises a first crRNA sequence, 4-10 consecutive thymidines, a second inverted crRNA sequence, 4-10 consecutive adenines, and a third inverted direct repeat sequence.

Another aspect of the invention includes a gene editing system capable of inducible, sequential mutagenesis in a cell comprising a plurality of vectors and a Cre recombinase. The vectors comprising a first promoter, a Cpf1 sequence, a second promoter, a first Cpf1 direct repeat sequence, a lox66 sequence, a second Cpf1 direct repeat sequence, two inverted restriction sites, an inverted lox71 sequence, and a crRNA FlipArray, wherein the crRNA FlipArray comprises a first crRNA sequence, 4-10 consecutive thymidines, a second inverted crRNA sequence, 4-10 consecutive adenines, and a third inverted direct repeat sequence.

In any of the gene editing systems of the present invention, the first crRNA and/or the second crRNA can target more than one sequence.

In another aspect, the invention includes a method of inducible, sequential mutagenesis in a cell. The method comprises administering to the cell a vector comprising a first promoter, a Cpf1 sequence, a second promoter, a first Cpf1 direct repeat sequence, a lox66 sequence, a second Cpf1 direct repeat sequence, two inverted restriction sites, an inverted lox71 sequence, and a crRNA FlipArray, wherein the crRNA FlipArray comprises a first crRNA sequence, 4-10 consecutive thymidines, a second inverted crRNA sequence, 4-10 consecutive adenines, and a third inverted direct repeat sequence. The first crRNA is expressed, then a Cre recombinase is administered to the cell. When the Cre recombinase is administered, the second crRNA is expressed, thus sequentially mutagenizing the cell.

Another aspect of the invention includes a method of inducible, sequential mutagenesis in a cell comprising administering to the cell a plurality of vectors. The vectors individually comprise a first promoter, a Cpf1 sequence, a second promoter, a first Cpf1 direct repeat sequence, a lox66 sequence, a second Cpf1 direct repeat sequence, two inverted restriction sites, an inverted lox71 sequence, and a crRNA FlipArray, wherein the crRNA FlipArray comprises a first crRNA sequence, 4-10 consecutive thymidines, a second inverted crRNA sequence, 4-10 consecutive adenines, and a third inverted direct repeat sequence. The first crRNA is expressed and a Cre recombinase is administered to the cell. When the Cre recombinase is administered, the second crRNA is expressed, thus sequentially mutagenizing the cell.

Yet another aspect of the invention includes a method of inducible, sequential mutagenesis in a cell in an animal. The method comprises administering to the animal a plurality of vectors. The vectors individually comprise a first promoter, a Cpf1 sequence, a second promoter, a first Cpf1 direct repeat sequence, a lox66 sequence, a second Cpf1 direct repeat sequence, two inverted restriction sites, an inverted lox71 sequence, and a crRNA FlipArray, wherein the crRNA FlipArray comprises a first crRNA sequence, 4-10 consecutive thymidines, a second inverted crRNA sequence, 4-10 consecutive adenines, and a third inverted direct repeat sequence. The first crRNA is expressed and a Cre recombinase is administered to the animal.

When the Cre recombinase is administered, the second crRNA is expressed, thus sequentially mutagenizing the cell in the animal.

In one embodiment of the method, the cell is a human cell. In one embodiment, the animal is a mouse. In one embodiment, the animal is a human. In one embodiment, mutagenesis is selected from the group consisting of nucleotide insertion, nucleotide deletion, frameshift mutation, gene activation, gene repression, and epigenetic modification. In one embodiment, the first crRNA and/or the second crRNA target more than one sequence. In one embodiment, the first crRNA targets Nf1 and the second crRNA targets Pten. In one embodiment, the first crRNA targets Pten and the second crRNA targets Nf1. In one embodiment, the first crRNA and/or the second crRNA targets a panel of immunomodulatory factors comprising Cd274, Ido1, B2m, Fas1, Jak2, and Lgals9

### CRISPR/Cpf1

As described herein, the discovery and characterization of the type V CRISPR system, Cpf1 (CRISPR from Prevotella and Francisella) has enabled rapid genome editing of multiple loci in the same cell. Cpf1 is a single component RNA-guided nuclease that can mediate target cleavage with a single crRNA. Compared to Cas9, Cpf1 does not require a tracrRNA, which greatly simplifies multiplexed genome editing of two or more loci simultaneously by using a string of crRNAs targeting different genes, as described herein. Thus, Cpf1 is an ideal system for high-throughput higher dimensional screens in mammalian species, with substantial advantages in library design and readout when compared to Cas9-based approaches. Herein, a Cpf1 crRNA array library that targets a set of the most significantly mutated cancer genes was designed. An unbiased screen was performed on two different mouse models, one studying early-stage tumorigenesis and the second studying cancer metastasis, identifying many unpredicted gene pairs. Thus, Cpf1 screening is a powerful approach to systematically quantify genetic interactions and identify new synergistic combinations. Unlike with Cas9-based strategies, due to the simple expansion of crRNA arrays, this approach can be readily extended to perform triple-, quadruple- or higher dimensional screens in vivo.

The Cpf1 enzyme can be derived from any genera of microbes, including but not limited to, Parcubacteria, Lachnospiraceae, Butyrivibrio, Peregrinibacteria, Acidaminococcus, Porphyromonas, Lachnospiraceae, Porphromonas, Prevotella, Moraxela, Smithella, Leptospira, Lachnospiraceae, Francisella, Candidatus, and Eubacterium. In certain embodiments, Cpf1 is derived from a species from the Acidaminococcus genus (AsCpf1). In other embodiments, Cpf1 is derived from a species from the Lachnospiraceae genus (LbCpf1). In yet other embodiments, the Cpf1 is a humanized form of LbCpf1.

In the context of formation of a CRISPR complex, “target sequence” refers to a sequence to which a crRNA sequence is designed to have some complementarity, where hybridization between a target sequence and a crRNA sequence promotes the formation of a CRISPR complex.

Full complementarity is not necessarily required, provided there is sufficient complementarity to cause hybridization and promote formation of a CRISPR complex. A target sequence may comprise any polynucleotide, such as DNA or RNA polynucleotides.

In certain embodiments, one or more vectors driving expression of one or more elements of a CRISPR system are introduced into a cell, such that expression of the elements of the CRISPR system direct formation of a CRISPR complex at one or more target sites. For example, a Cpf1 enzyme, and a crRNA could each be operably linked to separate regulatory elements on separate vectors. Alternatively, two or more of the elements expressed from the same or different regulatory elements may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction.

In certain embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity, and nucleic acid binding activity. Additional domains that can form part of a fusion protein comprising a CRISPR enzyme are described in U.S. Patent Appl. Publ. No. US20110059502, which is incorporated herein by reference. In certain embodiments, a tagged CRISPR enzyme is used to identify the location of a target sequence.

Conventional viral and non-viral based gene transfer methods can be used to introduce nucleic acids in mammalian and non-mammalian cells or target tissues. Such methods can be used to administer nucleic acids encoding components of a CRISPR system to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell (Anderson, 1992, Science 256:808-813; and Yu, et al., 1994, Gene Therapy 1:13-26).

In one non-limiting embodiment, a vector drives the expression of the CRISPR system. The art is replete with suitable vectors that are useful in the present invention. The vectors to be used are suitable for replication and, optionally, integration in eukaryotic cells. Typical vectors contain transcription and translation terminators, initiation sequences, and promoters useful for regulation of the expression of the desired nucleic acid sequence. The vectors of the present invention may also be used for nucleic acid standard gene delivery protocols. Methods for gene delivery are known in the art (U.S. Pat. Nos. 5,399,346, 5,580,859 & 5,589,466, incorporated by reference herein in their entireties).

Further, the vector can be provided to a cell in the form of a viral vector. Viral vector technology is well known in the art and is described, for example, in Sambrook et al. (4^{th} Edition, Molecular Cloning: A Laboratory Manual, Cold Spring Harbor Laboratory, New York, 2012), and in other virology and molecular biology manuals. Viruses, which are useful as vectors include, but are not limited to, retroviruses, adenoviruses, adeno-associated viruses, herpes viruses, Sindbis virus, gammaretrovirus, and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

Introduction of Nucleic Acids Methods of introducing nucleic acids into a cell include physical, biological and chemical methods. Physical methods for introducing a polynucleotide, such as RNA, into a host cell include calcium phosphate precipitation, lipofection, particle bombardment, microinjection, electroporation, and the like. RNA can be introduced into target cells using commercially available methods including electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany). RNA can also be introduced into cells using cationic liposome mediated transfection using lipofection, using polymer encapsulation, using peptide mediated transfection, or using biolistic particle delivery systems such as “gene guns” (see, for example, Nishikawa, et al., Hum Gene Ther., 12(8):861-70 (2001).

Biological methods for introducing a polynucleotide of interest into a host cell include use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Regardless of the method used to introduce exogenous nucleic acids into a host cell or otherwise expose a cell to the inhibitor of the present invention, in order to confirm the presence of the nucleic acids in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

It should be understood that the methods and compositions that would be useful in the present invention are not limited to the particular formulations set forth in the examples. The following examples are put forth so as to provide those of ordinary skill in the art with a complete disclosure and description, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, Molecular Cloning: A Laboratory Manual”, fourth edition (Sambrook et al. (2012) Molecular Cloning, Cold Spring Harbor Laboratory); “Oligonucleotide Synthesis” (Gait, M. J. (1984). Oligonucleotide synthesis. IRL press); “Culture of Animal Cells” (Freshney, R. (2010). Culture of animal cells. Cell Proliferation, 15(2.3), 1); “Methods in Enzymology” “Weir's Handbook of Experimental Immunology” (Wiley-Blackwell; 5 edition (Jan. 15, 1996); “Gene Transfer Vectors for Mammalian Cells” (Miller and Carlos, (1987) Cold Spring Harbor Laboratory, New York); “Short Protocols in Molecular Biology” (Ausubel et al., Current Protocols; 5 edition (Nov. 5, 2002)); “Polymerase Chain Reaction: Principles, Applications and Troubleshooting”, (Babar, M., VDM Verlag Dr. Müller (Aug. 17, 2011)); “Current Protocols in Immunology” (Coligan, John Wiley & Sons, Inc. Nov. 1, 2002).

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

The materials and methods employed in Experimental Examples 1-7 are now described.

Design, synthesis and cloning of the CCAS library: Significantly mutated genes (SMGs) were identified by analysis of pan-cancer mutation data of 17 cancer types from The Cancer Genome Atlas downloaded via Synapse (www dot synapse.org/#!Synapse:syni729383) and from the Broad Institute GDAC (gdac dot broadinstitute dot org/). The top 50 putative tumor suppressors (TSGs) were chosen in an unbiased manner using a multistep approach that prioritizes genes, which are significantly mutated in multiple cancer types and possess mutational signatures consistent with non-oncogenes. (1) A list of all significantly mutated genes in each of the 17 cancer types were first compiled by collecting all MutSig2CV results from GDAC and using a cutoff of q<0.1. (2) To remove putative oncogenes from the significantly mutated gene sets in each cancer type, the ratio of null to silent mutations for each SMG in that cancer was calculated, and this ratio was multiplied by the square root of the number of null mutations. (3) Ratio scores for each gene were then summed across cancer types. (4) Finally, to heavily weight genes that are SMGs in multiple cancer types, the summed ratio scores were multiplied by the number of unique cancer types in which a gene was considered an SMG. The resulting gene set was defined as PANCAN17-TSG50.

Of the top 50 putative TSGs identified by this approach, 49 were found to have clear mouse orthologs (defined as PANCAN17-mTSG). The complete exon sequences of these 49 genes were then analyzed to extract all possible Cpf1 spacers (i.e., all 20 mers beginning with the Cpf1 PAM, 5′-TTTN-3′). Each of these 20 mers was then reverse complemented and mapped to the entire mm10 reference genome by Bowtie 1.1.2, with settings bowtie -n 2-l 18 -p 8 -a -y --best -e 90. After filtering out all alignments that contained mismatches in the final 3 basepairs (corresponding to the Cpf1 PAM) and disregarding any mismatches in the fourth to last basepair, the number of genome-wide alignments for each crRNA were quantified using all 0, 1, and 2 mismatch (mm) alignments. A total mismatch score (MM score) was calculated for each crRNA using the following ad hoc formula: MM score=0 mm*1000+1 mm*50+2 mm*1. An “on-target” (OT) score was also approximated by counting the number of consecutive thymidines in each crRNA, and then using the formula: OT score=100/(max_consecutive_Thymidines)². All the crRNAs corresponding to each target gene were sorted by low MM score and high OT score. Finally, the top 2 crRNAs for each gene were chosen. In the event of ties, crRNAs targeting constitutive exons and/or the first exon were prioritized. 3 NTC crRNAs were randomly generated.

To generate the 9,408 DKO crRNA arrays in the library, all possible permutations of the 98 gene-targeting crRNAs were computed, with the stipulation that crRNAs targeting the same gene would not be included in the same crRNA array. For SKO crRNA arrays, each gene-targeting crRNA was placed in the first position of the crRNA array and the 3 NTCs were toggled through the second position (98*3=294 crRNA arrays). Finally, 3 NTC-NTC crRNA arrays were generated from various combinations of the 3 NTC single crRNAs.

Cell lines: A non-small cell lung cancer (NSCLC) cell line (KPD cell line) was used for initial testing of crRNA array constructs. An immortalized, but non-transformed hepatocyte cell line (clone IM) was transduced with LentiCpf1 to generate Cpf1-positive cells (IM.C9-Cpf1). All cell lines were grown under standard conditions using DMEM containing 10% FBS, 1% Pen/strep in a 5% CO₂ incubator.

Nextera analysis of indels generated by Cpf1: CrRNA arrays (crPten.crNf1 and crNf1.crPten) were cloned into Lenti-U6-crRNA vector, and virus was generated for transduction of KPD cell line.

(SEQ ID NO: 9,709)
crPten = TGCATACGCTATAGCTGCTT
(SEQ ID NO: 9,710)
crNfl = TAAGCATAATGATGATGCCA

Seven days after transduction and puromycin selection, genomic DNA was harvested from the cells in culture. The surrounding genomic regions flanking the target sites of crPten and crNf1 were first amplified by PCR using the following primers (5′-3′): Pten_fwd=ACTCACCAGTGTTTAACATGCAGGC (SEQ ID NO: 9,711), Pten_rev=GGCAAGGTAGGTACGCATTTGCT (SEQ ID NO: 9,712); Nf1_fwd=AGCAGCTGTCCTGGCTGTTC (SEQ ID NO: 9,713), Nf1_rev=CGTGCACCTCCCTTGTCAGG (SEQ ID NO: 9,714). Nextera XT library preparation was then performed according to manufacturer protocol. Reads were mapped to the mm10 mouse genome using BWA (Li and Durbin. Bioinforma. Oxf Engl. 25, 1754-1760 (2009)), with the settings bwa mem -t 8 -w 200. Indel variants were first processed with Samtools (Li, H. et al. Bioinformatics 25, 2078-2079 (2009)) with the settings samtools mpileup -B -q 10 -d 10000000000000, then piped into VarScan v2.3.9 (Koboldt, et al. Genome Res. 22, 568-576 (2012)) with the settings pileup2indel --min-coverage 1 --min-reads2 1 --min-var-freq 0.00001.

Lentiviral library production: The LentiCpf1, Lenti-U6-crRNA vector and Lenti-CCAS library plasmids were used to make vector or library-containing lentiviruses. Briefly, envelope plasmid pMD2.G, packaging plasmid psPAX2, and LentiCpf1, Lenti-U6-crRNA or Lenti-CCAS-library plasmid were added at ratios of 1:1:2.5, and then polyethyleneimine (PEI) was added and mixed well by vortexing. The solution was standing at room temperature for 10-20 min, and then the mixture was dropwisely added into 80-90% confluent HEK293FT cells and mixed well by gently agitating the plates. Six hours post-transfection, fresh DMEM supplemented with 10% FBS and 1% Pen/Strep was added to replace the transfection media. Virus-containing supernatant was collected at 48 h and 72 h post-transfection, and was centrifuged at 1500 g for 10 min to remove the cell debris; aliquoted and stored at −80° C. Virus was titrated by infecting IM-Cpf1 cells at a number of different concentrations, followed by the addition of 2 μg/mL puromycin at 24 h post-infection to select the transduced cells. The virus titers were determined by calculating the ratios of surviving cells 48 or 72 h post infection and the cell count at infection.

CCAS in a mouse model of transformation and early tumorigenesis: Cells were transduced and library transduction was performed with four infection replicates at high coverage and low MOI. Briefly, according to the viral titers, CCAS library lentiviruses were added into a total of >1×10⁸ IM.C9-Cpf1 cells at calculated MOI of <=0.2 and incubated 24 h before replacing the viruses-containing media with 3 g/mL puromycin containing fresh media to select the virus-transduced cells. Approximately 2×10⁷ cells confer a -2,000× library coverage. Vector and CCAS library-transduced cells were culture under the pressure of 3 μg/mL puromycin for 7 days before injection or cryopreservation.

Vector and CCAS library-transduced IM.C9-Cpf1 cells were injected subcutaneously into the right and left flanks of Nu/Nu mice at 4×10⁶ cells per flank (˜400× coverage per transplant). Tumors were measured every week by caliper and their sizes were estimated as spheres. Statistical significance was assessed by paired t-test.

Mouse tumor dissection and histology: Mice were sacrificed by carbon dioxide asphyxiation followed by cervical dislocation. Tumors and other organs were manually dissected, and then fixed in 10% formalin for 24-96 hours, and transferred into 70% Ethanol for long-term storage. The tissues were embedded in paraffin, sectioned at 5 μm and stained with hematoxylin and eosin (H&E) for pathological analysis. For tumor size quantification, H&E slides were scanned using an Aperio digital slidescanner (Leica). For molecular biological analysis, tissues were flash frozen with liquid nitrogen, and ground in 5 mL Frosted polyethylene vial set (2240-PEF) in a 2010 GenoGrinder machine (SPEXSamplePrep). Homogenized tissues were used for DNA/RNA/protein extractions.

CCAS in a mouse model of metastasis: For Cpf1 crRNA array library screen in a mouse model of metastasis, lentiviral pools were generated from the CCAS plasmid library, and transduced ≥1×10⁸ Cpf1+ KPD cells with three independent infection replicates at calculated MOI of ≤0.2 and incubated 24 h before replacing the viruses-containing media with 3 g/mL puromycin containing fresh media to select the virus-transduced cells. Approximately 2×10⁷ cells confer a 2,000× library coverage. CCAS library-transduced cells were culture under the pressure of 3 μg/mL puromycin for 7 days before injection or cryopreservation.

CCAS-treated cells were then injected at 4×10⁶ cells per mouse (˜400× coverage) subcutaneously into Nu/Nu mice (n=7) and Rag1−/− mice (n=4). Metastases were allowed to form in vivo for 8 weeks after injection. Primary tumors, four lung lobes, and other stereoscope-visible metastases, were then dissected and then subjected to genomic DNA extraction and crRNA array sequencing.

Genomic DNA extraction: 200-800 mg of frozen ground tissue were re-suspended in 6 mL of NK Lysis Buffer (50 mM Tris, 50 mM EDTA, 1% SDS, pH 8.0) supplemented with 30 μL of 20 mg/mL Proteinase K (Qiagen) in 15 mL conical tubes, and incubated at 55° C. bath for 2 h up to overnight. After all the tissues have been lysed, 30 μL of 10 mg/mL RNAse A (Qiagen) was added, mixed well and incubated at 37° C. for 30 min. Samples were chilled on ice and then 2 mL of pre-chilled 7.5 M ammonium acetate (Sigma) was added to precipitate proteins. The samples were inverted and vortexed for 15-30 s and then centrifuged at ≥4,000 g for 10 min. The supernatant was carefully decanted into a new 15 mL conical tube, followed by the addition of 6 mL 100% isopropanol (at a ratio of 0.7), inverted 30-50 times and centrifuged at ≥4,000 g for 10 minutes. Genomic DNA should be visible as a small white pellet. After discarding the supernatant, 6 mL of freshly prepared 70% ethanol was added, mixed well, and then centrifuged at ≥4,000 g for 10 min. The supernatant was discarded by pouring; and remaining residues was removed using a pipette. After air-drying for 10-30 min, DNA was re-suspended by adding 200-500 μL of Nuclease-Free H₂O. The genomic DNA concentration was measured using a Nanodrop (Thermo Scientific), and normalized to 1000 ng/L for the following readout PCR.

Cpf1 CrRNA array library readout: The crRNA array library readout was performed using a 2-step PCR approach. Briefly, in the 1st round PCR, enough genomic DNA was used as template to guarantee coverage of the library abundance and representation. For example, assuming 6.6 pg of gDNA per cell, 20-48 μg of gDNA (≥75×) was used per sample. For the 1st PCR, the sgRNA-included region was amplified using primers specific to the double-knockout CCAS vector using Phusion Flash High Fidelity Master Mix (ThermoFisher) with thermocycling parameters: 98° C. for 1 min, 15 cycles of (98° C. for is, 60° C. for 5 s, 72° C. for 15s), and 72° C. for 1 min. Fwd: AATGGACTATCATATGCTTACCGTAACTTGAAAGTATTTCG (SEQ ID NO: 9,715); Rev: CTTTAGTTTGTATGTCTGTTGCTATTATGTCTACTATTCTTTCCC (SEQ ID NO: 9,716)

In the 2nd PCR, 1st round PCR products for each biological repeats were pooled, then 1-2 μL well-mixed 1st PCR products were used as the template for amplification using sample-tracking barcode primers with thermocycling conditions as 98° C. for 1 min, 15 cycles of (98° C. for is, 60° C. for 5 s, 72° C. for 15s), and 72° C. for 1 min. The 2^{nd} PCR products were quantified in 2% E-gel EX (Life Technologies) using E-Gel® Low Range Quantitative DNA Ladder (ThermoFisher), then the same amount of each barcoded samples were combined. The pooled PCR products were purified using QIAquick PCR Purification Kit and further QIAquick Gel Extraction Kit from 2% E-gel EX. The purified pooled library was quantified in a gel-based method. Diluted libraries with 5-20% PhiX were sequenced with Hiseq 2500 or HiSeq 4000 systems (Illumina) with 150 bp paired-end read length.

Cpf1 double knockout Illumina data pre-processing: Raw single-end fastq read files were filtered and demultiplexed using Cutadapt (Martin, EMBnet.journal 17, 10-12 (2011)). To remove extra sequences downstream (i.e. 3′ end) of the dual-RNA spacer sequences, including the U6 terminator, the following settings were used: cutadapt --discard-untrimmed -e 0.1 -a TTTTTTAAGCTTGGCGTGGATCCGATATCA (SEQ ID NO: 9,717). As the forward PCR primers used to readout crRNA array representation were designed to have a variety of barcodes to facilitate multiplexed sequencing, these filtered reads were then demultiplexed with the following settings: cutadapt -g file:fbc.fasta --no-trim, where fbc.fasta contained the 12 possible barcode sequences within the forward primers. Finally, to remove extraneous sequences upstream (i.e. 5′ end) of the crRNA array spacers, including the first DR, the following settings were used: cutadapt --discard-untrimmed -e 0.1 -g AAAGGACGAAACACCgTAATTTCT ACTAAGTGTAGAT (SEQ ID NO: 9,718). Through this procedure, the raw fastq read files were pared down to the sequences of the first crRNA, the second DR, and finally the second crRNA (cr1-DR-cr2). The filtered fastq reads were then mapped to the CCAS reference index.

To do so, a bowtie index of the CCAS library was first generated using the bowtie-build command in Bowtie 1.1.2 (Langmead, et al. (2009), Genome Biol. 10, R25). Using these bowtie indexes, the filtered fastq read files were mapped using the following settings: bowtie -v 2 -k 1 -m 1 --best. These settings ensured only single-match reads would be retained for downstream analysis.

Analysis of CCAS library representation: Using the resultant mapping output, the number of reads that had mapped to each crRNA array within the library were quantitated. The number of reads in each sample were normalized by converting raw crRNA array counts to reads per million (rpm). The rpm values were then subject to log₂ transformation for certain analyses. To generate correlation heatmaps, the NMF R package was used. To generate sgRNA representation barplots, a detection threshold of log₂ rpm≥1 was set, and the number of unique crRNA arrays present in each sample was counted.

Analysis of enriched DKO and SKO crRNA arrays: To directly compare the abundance in tumor samples vs. cells, linear regression was performed and significant outliers identified using the outlierTest function from the car R package. Significant outlier crRNA arrays in individual tumors vs. cells were defined as having a Bonferroni adjusted p<0.05, based on analysis of the studentized regression residuals.

To identify crRNA arrays significantly enriched above NTC-NTC controls, two-sided t-tests were similarly performed on the log₂ rpm abundance of each crRNA array compared to the average of all NTC-NTC crRNA arrays. Significantly enriched crRNA arrays were defined as having a Benjamini-Hochberg adjusted p<0.05. Each significantly enriched crRNA array was then deconstructed into its two constituent crRNAs, and finally down to the two target genes. This 3-tiered dataset was used to determine how many genes were involved in an enriched crRNA array (either SKO or DKO). Finally, all of the significant crRNA arrays associated with each gene were compiled, and the number of DKO or SKO crRNA arrays counted.

Position effect analysis of crRNA permutations: Marginal distribution analysis was performed by considering each of the 98 single crRNAs when found in position 1 or position 2 of the crRNA array. Specifically, the average log₂ rpm abundance was calculated for each single crRNA, and these average scores were compared between position 1 and position 2. For direct permutation correlation analysis, the 9,408 DKO crRNA arrays were condensed down into 4,704 crRNA array combinations (i.e., crX.crY and crY.crX are two permutations of the same combination). The correlation between the two corresponding permutations was then calculated the across all 10 tumor samples (defined as permutation correlation), and the statistical significance assessed by t-distribution. Violin plots, empirical density plots, and scatterplots were generated using these permutation correlation coefficients.

Synergy analysis of gene pairs: The synergy coefficient (SynCo) for each DKO crRNA array was defined with the following formula:

SynCo=DKO_{xy}−SKOₓ−SKO_{y}

The DKOₓy score is the log₂ rpm abundance of the DKO crRNA array (i.e., crX.crY) after subtracting average NTC-NTC abundance, while SKOₓ and SKO_{y} scores are defined as the average log₂ rpm abundance of each SKO crRNA array (3 SKO crRNA arrays associated with each individual crRNA), each after subtracting average NTC-NTC abundance. By this definition, a SynCo score>>0 would indicate that a given DKO crRNA array is synergistic, as the DKO score would thus be greater than the sum of the individual SKO scores. The SynCo of each DKO crRNA array was calculated within each tumor sample and it was assessed whether the SynCo score of a given crRNA array across all 10 tumors was statistically significantly different from 0 by a two-sided one-sample t-test. A significance threshold of Benjamini-Hochberg adjusted p<0.05 was set, and all significant DKO crRNA arrays with an average SynCo>0 were considered to be synergistic.

Network analysis: Using the synergistic crRNA arrays identified through SynCo analysis, library-wide networks were constructed using individual genes as nodes and SynCo scores as edge weights. The pairwise connections were visualized through Cytoscape 3.4.0 (Shannon et al., Genome Res. 13, 2498-2504 (2003)). Edge width was scaled according to SynCo score. For the global network, node color was additionally scaled according to the degree of network connectivity.

Analysis of co-mutation patterns in human pan-cancer datasets: For the synergistic driver pairs identified by the CCAS screen, co-mutation analyses were performed on 21 different solid tumor types, all of which were from TCGA except for small cell lung cancer. The somatic mutation and copy number status of each cohort were obtained from cBioPortal (Cerami et al., Cancer Discov. 2, 401-404 (2012) (only somatic mutations were available for lung small cell cancer) and classified all tumors as a mutant or non-mutant for the genes represented in the CCAS library. “Mutant” was defined as the presence of nonsynonymous mutations and/or deep deletions in a given gene. After classifying every patient in terms of mutant status, co-mutation (co-occurrence) analysis was performed by calculating the co-occurrence rate for each gene pair. The co-occurrence rate was defined as the intersection (the number of double mutant samples) divided by the union (the number of all single and double mutant samples). Statistical significance was tested by a hypergeometric test, with a significance threshold of Benjamini-Hochberg adjusted p<0.05.

Analysis of metastasis enrichment over primary tumor and metastatic clonal spread: Comparison of the crRNA array representations was made between metastases to primary tumors. A crRNA array was called metastasis-enriched if it was a dominant clone in a lung lobe or extra-pulmonary metastasis (≥2% total reads) but not a dominant clone in the corresponding primary tumor of the same mouse. Waterfall plot was made for all crRNA arrays enriched in a metastases vs primary tumor, ranked by numbers of mice where an crRNA was called enriched.

Monoclonal spread was defined where dominant metastases in all lobes were derived from identical crRNA arrays, and polyclonal spread was defined where dominant metastases in all lobes were derived from multiple varying crRNAs.

Blinding statement: Investigators were blinded for sequencing data analysis, but not blinded for tumor engraftment, organ dissection and histology analysis.

The results of the experiments from Examples 1-7 are now described.

### Example 1: Enabling One-Step Double Knockout Screening with a Cpf1 crRNA Array Library

To establish a lentiviral system for CRISPR/Cpf1-mediated genetic screening, a human-codon-optimized LbCpf1 expression vector (pLenti-EFS-Cpf1-blast, LentiCpf1 for short) and a crRNA expression vector (pLenti-U6-DR-crRNA-puro, Lenti-U6-crRNA for short) were generated (FIG. 1A). In order to facilitate direct and targeted double knockout studies using a single crRNA array, oligos were designed with a 5′ homology arm to the base vector, followed by a crRNA, the direct repeat (DR) sequence for Cpf1, a second crRNA, a U6 terminator, and finally a 3′ homology arm (cr1-DR-cr2). As the oligos each contained two crRNAs, these constructs were termed crRNA arrays. Linearization of the Lenti-U6-crRNA vector enabled one-step cloning of the crRNA array into the vector by Gibson assembly, producing the double knockout crRNA array expression vector (pLenti-U6-DR-cr1-DR-cr2-puro) (FIG. 1B). The constructs were tested for their ability to induce double knockouts in a murine cancer cell line (KPD) in vitro. After infection with LentiCpf1, the cells were transduced with lentiviruses carrying a crRNA array targeting Pten and Nf1 (FIG. 8A). To confirm whether Cpf1 can mediate mutagenesis regardless of the position of each crRNA within the array, two permutations of the Pten and Nf1 crRNA array were generated (crPten.crNf1 and crNf1.crPten, all with 20 nt spacers) (FIG. 8A). Both crPten.crNf1 and crNf1.crPten crRNA arrays generated indels at both loci in Cpf1+ KPD cells (FIG. 8B). These data confirmed that a single crRNA array can be used in conjunction with CRISPR-Cpf1 to mediate simultaneous knockout of two genes in mammalian cells.

To investigate whether Cpf1 multiplex gene targeting could be utilized for multidimensional genetic interaction screens, a library for Cpf1 crRNA array screening was developed (CCAS library). Considering the resolution of library complexity under in vivo cellular dynamics, a focused CCAS library was designed of the top 50 significantly mutated genes (SMGs) that are not oncogenes, with the vast majority being established or putative tumor suppressor genes (TSGs) identified through analysis of 17 different cancer types from The Cancer Genome Atlas (TCGA). The resultant gene set was termed PANCAN7-TSG50. (FIG. 1C). 49 of the PANCAN17-TSG50 genes had corresponding mouse orthologs (PANCAN17-mTSG), and were thus included in the CCAS library. All possible Cpf1 spacer sequences were identified within PANCAN17-mTSG and subsequently 2 crRNAs were chosen for each gene. The selection of crRNAs was based on two scoring criteria: (1) high genome-wide mapping specificity and (2) a low number of consecutive thymidines, since long stretches of thymidines will terminate U6 transcription.

Compiling these 98 gene-targeting crRNAs and 3 additional non-targeting control (NTC) crRNAs, crRNA array library was designed containing 9,705 permutations of two crRNAs each (FIGS. 20A-20F). Of the 9,705 total crRNA arrays in the library (SEQ ID NOs: 4-9,708), 9,408 were comprised of two gene-targeting crRNAs (double knockout, or DKO), while 294 contained one gene-targeting crRNA and one NTC crRNA (single knockout, or SKO). The remaining 3 crRNA arrays were dedicated controls, with two different NTC crRNAs in the crRNA array (NTC-NTC). After pooled oligo synthesis, the PANCAN17-mTSG CCAS library was cloned into the base vector, and the plasmid crRNA array representation subsequently readout by deep-sequencing the crRNA expression cassette. All 9,705/9,705 (100%) of the designed crRNA arrays were successfully cloned (FIG. 1D, FIG. 9B). Analysis of each crRNA array within the CCAS library revealed that the relative abundances of both DKO and SKO crRNA arrays approximated a log-normal distribution, demonstrating even coverage of the CCAS library (FIG. 1D). Lentiviral pools from the CCAS plasmid library were generated for subsequent high-throughput double-mutagenesis and genetic interaction screens.

### Example 2: Library-Scale Cpf1 crRNA Array Screen in a Mouse Model of Early Tumorigenesis

To perform an in vivo Cpf1 screen, a mouse model of malignant transformation and early stage tumorigenesis was utilized. An immortalized murine cell line was transduced with low tumorigenicity (clone IM) with LentiCpf1 and then with the CCAS lentiviral pool. The library transduction was performed with four infection replicates at high coverage (˜2,000× coverage for each replicate) and low multiplicity of infection (MOI, ≤0.2) to ensure the vast majority of cells would only carry one provirus integrant (FIG. 2A). After 7 days of puromycin selection, only CCAS-virus infected cells survived, comprising a mixture of various double mutants (termed CCAS-treated cells hereafter). In parallel, another group of Cpf1+ cells were infected with lentiviruses carrying the empty vector. The virus-treated cell populations were then subcutaneously injected into nude mice (CCAS, n=10 mice; vector, n=4 mice). By 45 days post-injection (dpi), CCAS-treated cells had given rise to significantly larger tumors than vector-treated cells (p=0.0223, by two-sided t-test) (FIG. 2B). This trend continued through the duration of the experiment (46.5 dpi, p=0.0017). A select fraction of tumors derived from CCAS-treated cells were harvested and sectioned for histological analysis, together with the small nodules derived from vector-treated cells (FIG. 2C).

To unveil the genetic interactions that had driven rapid tumor growth upon Cpf1-mediated mutagenesis, crRNA array sequencing was performed on genomic DNA from CCAS tumors (n=10) and pre-injection cell pools (n=4). Whereas plasmid and cell samples were highly correlated with one another, tumor samples were more correlated with other tumors (FIG. 9A). All plasmid and cell samples contained 100% of CCAS crRNA arrays, while tumor samples exhibited significantly lower crRNA array library diversity (mean SEM=37.0% 10.5%; p 2.02 e-4 compared to plasmid and cells, t-test) (FIG. 9B). Furthermore, while plasmid and cell samples exhibited robust lognormal representation of the CCAS library (FIG. 9C), tumor samples showed strong enrichment of specific SKO and DKO crRNA arrays (FIG. 9C, FIG. 2D). Of note, the 3 NTC-NTC controls in the CCAS library were consistently found at abundances similar to one another within each of the plasmid and cell samples. All NTC-NTCs were found at low abundance across all tumor samples (average log₂ rpm abundance=0.224±0.108), suggesting non-mutagenized cells do not have a selective advantage in tumorigenesis and that additional genetic perturbation is needed to drive rapid tumor growth in vivo. As a global comparison of all crRNA arrays, while the mean abundance in tumor samples correlated with the mean abundance in cell samples in a log-linear manner (regression r²=0.166, coefficient=0.569, p<2.2 e-16 by F-test), a population of crRNA arrays were outliers (Outlier test, Bonferroni adjusted p<0.05), indicating that specific crRNA arrays had undergone positive selection in vivo (FIG. 2E). This trend was consistent across the individual tumors (p<2.2 e-16 by F-test for all individual tumors, average number of outliers compared to cells=102.1±3.671 crRNA arrays) (FIGS. 10A-10B). Taken together, these data suggest that a subgroup of crRNA arrays was enriched in tumors, indicating that a select number of mutant clones had significantly expanded in vivo.

### Example 3: Enrichment Analysis of Single Knockout and Double Knockout crRNA Arrays

To further investigate the specific genetic interactions that had driven early stage tumorigenesis in CCAS-treated cells, the distribution of raw crRNA array abundance within each sample was examined. Within each tumor, specific crRNA arrays were observed that were heavily enriched by several orders of magnitude, suggesting that these mutant clones had undergone potent positive selection (FIG. 3A, FIG. 9D). For example, in Tumor 1, crCasp8.crApc was by far the most abundant crRNA array, dwarfing all other crRNA arrays including the corresponding SKO crRNA arrays crApc.NTC and crCasp8.NTC (FIG. 3A).

Interestingly, this finding that several DKO crRNA arrays were more heavily enriched than their SKO counterparts was corroborated across tumors. For instance, Tumor 3 was dominated by crSetd2.crAcvr2a and crRnf43.crAtrx, Tumor 5 by crCic.crZc3h13 and crCbwd1.crNsd1, and Tumor 6 by crAtm.crRunx1 and crKmt2d.crH2-Q2 (FIG. 3A, FIG. 9D). In all of these cases, the corresponding SKO crRNA arrays were far less abundant compared to the DKO crRNA arrays.

Taken together, these data point to the dominance of a handful of individual clones within each tumor sample, and further suggest that certain double-mutant clones had out-competed the corresponding single-mutant clones.

In order to uncover the genetic interactions underlying the positive selection in vivo, the next set of experiments set out to quantitatively identify all significantly enriched crRNA arrays across all 10 tumors. The abundance of each DKO and SKO crRNA array was compared to the average of all NTC-NTC crRNA arrays. 655 crRNA arrays targeting 498 gene combinations were found to be significantly enriched compared to NTC-NTC controls (Benjamini-Hochberg adjusted p<0.05) (FIG. 3B). Of these, 620 were DKO crRNA arrays and 35 were SKO crRNA arrays. The 655 significantly enriched crRNA arrays were decomposed to their constituent single crRNAs, and the target genes associated with each single crRNA were identified. All 49 genes in the PANCAN17-mTSG CCAS library were represented within at least one significant DKO crRNA array, and 24 genes were additionally found to be significant as part of a SKO crRNA array (FIG. 3B). To identify the genes most frequently targeted among the set of 655 significant crRNA arrays, the number of significant crRNA arrays associated with each gene were counted. Rnf43 and Kmt2c were the two genes with the largest number of significant crRNA arrays (FIG. 3C). Interestingly, of the top 10 genes in this analysis, 6 are epigenetic modifiers (Kmt2c, Atrx, Kdm5c, Setd2, Kdm6a, and Arid1a), revealing the direct phenotypic consequence of their loss-of-function in tumor suppressor gene networks.

Specific genetic interactions that comprise this network were then investigated. The number of significant DKO crRNA arrays associated with each gene pair were quantified (FIG. 3D). 113 gene pairs were represented by at least 2 independent DKO crRNA arrays. Strikingly, the interaction of Atrx+Setd2 was supported by 5 independent crRNA arrays, while Atrx+Kmt2c, Arid1a+Map3k1, Kdm5c+Kmt2c, and Arid1a+Rnf43 were substantiated by 4 crRNA arrays. In aggregate, these analyses generated an unbiased profile of genetic interactions in tumor suppression dismantled upon Cpf1-mediated double-mutagenesis.

To investigate possible positional effects for each individual crRNA in the CCAS library, the two permutations of each crRNA array combination were directly compared (FIG. 11A). For each of the 4,704 DKO crRNA array combinations (condensed from 9,408 DKO crRNA array permutations), the Pearson correlation of crRNA array abundance was calculated (i.e. comparing crX.crY to crY.crX) across all tumor samples (subsequently referred to as permutation correlation). Examining the distribution of permutation correlations, a strong skew towards high correlation coefficients was observed (median permutation correlation >0.97) (FIG. 3E), indicating that for most crRNA array combinations, the positioning of constituent single crRNAs did not affect in vivo abundance of the crRNA array (FIG. 11B). In total, 80.1% (3,767/4,704) of all crRNA array combinations were significantly correlated when comparing the 2 permutations associated with each combination (Benjamini-Hochberg adjusted p<0.05, by t-distribution). The two most significantly correlated crRNA array combinations were between the single crRNAs crH2-Q2.1 and crPten.240, and between crCbwd1.84 and crEpha2.5. CrH2-Q2.1_crPten.240 was strongly correlated with the abundance of crPten.240_crH2-Q2.1 across all 10 tumors (R=0.999, p=2.28 e-19), and a similar trend was observed between crCbwd1.84_crEpha2.5 and crEpha2.5_crCbwd1.84 (R=0.999, p=7.09 e-19) (FIGS. 11C-11D).

To quantitate the gross contributions of individual crRNAs to tumorigenesis, marginal distribution meta-analysis of all 98 constituent single crRNAs in the CCAS library was performed (FIG. 11E). As the CCAS library was designed with crRNA array orientation as a consideration (FIG. 1C), the average log₂ rpm abundance of all DKO crRNA arrays associated with each single crRNA when present in position 1 or in position 2 of the crRNA array was calculated. Across all 98 single crRNAs, the average abundance for each single crRNA when in position 1 was significantly correlated with its average abundance when in position 2 (Pearson correlation coefficient (R)=0.397, p=5.25 e-5 by t-distribution). This finding suggests that a crRNA confers a similar selective advantage regardless of position in the crRNA array considering all other crRNAs it is paired with.

### Example 4: High-Throughput Identification of Synergistic Drivers of Transformation and Tumorigenesis

To quantitatively investigate the genetic interactions in this model, a metric of synergy for DKO crRNA arrays was developed. Since the relative abundance of a crRNA array is effectively an estimate of its relative selective advantage in vivo, the synergy coefficient (SynCo) for each DKO crRNA array was defined as DKO_{y}−SKOₓ−SKO_{y}. The DKOₓ score is the log₂ rpm abundance of the DKO crRNA array (i.e., crX.crY) after subtracting average NTC-NTC abundance; SKOₓ and SKO_{y} scores are defined as the average log₂ rpm abundance of each SKO crRNA array (3 SKO crRNA arrays associated with each individual crRNA), each after subtracting average NTC-NTC abundance (FIG. 4A). By this definition, a SynCo score>>0 would indicate that a given DKO crRNA array is synergistic, as the DKO score would thus be greater than the sum of the individual SKO scores on a log-linear scale.

The SynCo of each DKO crRNA array within each tumor sample was calculated, and it was assessed whether the SynCo score of a given crRNA array across all 10 tumors was statistically significantly different from 0 by a two-sided one-sample t-test. Out of 9,408 DKO crRNA arrays, 294 were significantly synergistic (Benjamini-Hochberg adjusted p<0.05, average Synco>0), representing 270 gene combinations. To obtain a comprehensive picture of the synergistic driver pairs, the average SynCo of each DKO crRNA array was plotted against its associated p-value, while additionally color-coding each point by average abundance and scaling the size of each point by the percentage of tumors that had a high SynCo score (SynCo>7) for that crRNA array (FIG. 4B). Among the top synergistic driver pairs in this analysis were crSetd2.crAcvr2a and crCbwd1.crNsd1. Setd2 encodes a histone methyltransferase that has been implicated in a number of cancer types, while Acvr2a is a receptor serine-threonine kinase that plays a critical role in Tgf-P signaling and is frequently mutated in microsatellite-unstable colon cancers. Nsd1 encodes a lysine histone methyltransferase that has been linked to Sotos syndrome, a genetic disorder of cerebral gigantism, and has been implicated in various cancers. In contrast, Cbwd1 encodes an evolutionarily conserved protein whose biological function is unknown; on the basis of its amino acid sequence, Cbwd1 has been predicted to contain a cobalamin synthase W domain, but its function has never been characterized in a mammalian species. Interestingly, many of the high-score SynCo-significant gene pairs have not been functionally characterized in literature.

To pinpoint the most robust genetic interactions from SynCo analysis, the number of synergistic dual-crRNAs associated with each gene pair was quantified. Of the 268 significant gene pairs, 24 were represented by at least 2 synergistic dual-crRNAs (FIG. 4C). Considering that many gene pairs might have additive effects, the SynCo score is a stringent metric of genetic interaction; thus the finding that several gene pairs were further supported by multiple synergistic dual-crRNAs provides further evidence for the genetic interactions between these genes.

Two hundred and seventy significant pairwise genetic interactions in early tumorigenesis were identified, many of which corresponded to genomic features of human tumors. Next, each of these gene pairs was placed within the larger network of tumor suppression. A network of all synergistic driver interactions captured by CCAS screening was constructed, where each node represented a gene and each edge represented a significant synergistic interaction (FIG. 12). In this network, the color of each gene was scaled by its degree of connectivity, while edge widths were scaled to the SynCo score associated with that interaction. Surprisingly, H2-Q2, a gene encoding a major histocompatibility complex (MHC) component, the murine homolog of human HLA-A MHC class I A, was found to have the greatest network connectivity, with 19 different interacting partners (FIG. 4D). Of note, H2-Q2 shared its strongest interaction with Kmt2d (SynCo=8.877), pointing to a genetic interaction between an epigenetic modifier and an immune regulator in tumorigenesis. Many of these synergistic pairs were significantly co-mutated in one or more cancer types (top 50 SynCo interactions shown in FIG. 4E), suggesting relevance of these genomic features in human cancers.

### Example 5: Cpf1 crRNA Array Library Screen in a Mouse Model of Metastasis

Cpf1 crRNA array library screening was performed in a mouse model of metastasis to identify co-drivers of the metastatic process in vivo. Lentiviral pools were generated from the CCAS plasmid library, and Cpf1+ KPD cells were subsequently infected to perform massively parallel gene-pair level mutagenesis. The mixed double mutant cell populations (CCAS-treated cells, 4×10⁶ cells per mouse, ˜400× coverage) were then injected subcutaneously into Nu/Nu mice (n=7) and Rag1−/− mice (n=4). After 8 weeks, the primary tumors, four lung lobes, and other stereoscope-visible metastases (two large extra-pulmonary metastases were found) were collected and subjected to crRNA array sequencing (FIG. 5A). The 3 pre-injection cell pools, as well as primary tumors and metastases from all 11 mice were sequenced. As seen in the overall representation of the CCAS library across all metastasis screen samples (FIG. 5B, FIG. 14), cell samples exhibited lognormal representation of the CCAS library, whereas both primary tumors and metastases showed strong enrichment of specific SKO and DKO crRNA arrays. NTC-NTC crRNA arrays were consistently found at low abundance in all primary tumors and metastases samples, indicating strong selection and clonal expansion during the metastasis process. Notably, the crRNA library representation of metastases in all the collected lobes showed high degree of similarity to primary tumors (FIG. 5C), consistent with a common clonal origin from the same primary tumors within each individual mouse.

### Example 6: Enrichment Analysis of crRNA Arrays Identified Metastasis Drivers and Co-Drivers

In the CCAS metastasis screen dataset, strong overall permutation correlation was observed, where 97.4% of all crRNA array combinations were significantly correlated when comparing the two permutations associated with each combination (Benjamini-Hochberg adjusted p<0.05, by t-distribution) (median permutation correlation >0.85) (FIG. 6A), indicating that for most crRNA array combinations, the positioning of constituent single crRNAs did not affect in vivo abundance of the crRNA array. DKO and SKO crRNA arrays were then compared to NTC-NTC controls in the metastasis screen. Across all in vivo samples, 2933 crRNA arrays were found to be significantly enriched compared to NTC-NTC controls (Benjamini Hochberg-adjusted p<0.05), targeting 1006 combinations. Of these, 2813 were DKO crRNA arrays and 121 were SKO crRNA arrays (FIG. 6B). All 49 genes in the PANCAN17-mTSG CCAS library were represented within at least one significant DKO crRNA array. The top 15 genes associated with these 2933 crRNA arrays ranked by the number of significant crRNA arrays associated with each gene were found to be Arid1a, Cdh1, Kdm5c, Rb1, Epha2, Kmt2b, Cic, Kmt2c, Kdm6a, Atra, Nf2, Elf3, Apc, Rnf43 and Ctcf (FIG. 6C).

Independent evidence for selection of metastasis co-drivers was sought via investigation of independent crRNA arrays targeting the same gene pair. By calculating the number of significant DKO crRNA arrays associated with each gene pair in the CCAS library, it was discovered that the majority (729/1176=61.99%) of gene pairs were represented by at least 2 independent DKO crRNA arrays. Of note, 30 gene pairs were represented by seven independent crRNA arrays, among them including Apc+Cdh1, Cdh1+H2-Q2, Epha2+Kmt2b; and 8 gene pairs were represented by all eight designed crRNA arrays, including Arid1a+Pten, Cdh+Nf1, Cdh1+Kdm5c, Arid1a+Rasa1, Arid1a+Cdh1 Cdh7+Kmt2b, Arid1a+Kmt2b, and Arid1a+Epha2, suggesting these are the strong co-drivers of metastasis (FIG. 6D).

### Example 7: Modes and Patterns of Metastatic Spread with Co-Drivers

The in vivo patterns of metastatic evolution of these double mutants were investigated. Examination of the clonal architecture of the crRNA arrays in the metastases samples revealed a highly heterogenous pattern of clonal dominance (FIGS. 15A-15G). Comparison of the crRNA array representations between metastases to primary tumors revealed modes of monoclonal spread (FIG. 7A, FIGS. 15A-15G) where dominant metastases in multiple lobes were derived from identical crRNA arrays, as well as polyclonal spread (FIG. 7B) and where dominant metastases in all lobes were derived from multiple varying crRNAs. For example, mouse 1 represents a case of monoclonal spread where all 4 lobes were dominated by a clone, crNf2.crRnf43, which was also found at the primary tumor as a major clone (>=2% frequency). In contrast, mouse 10 represents a case of polyclonal spread where each lung lobe was comprised of a myriad of crRNA arrays. Namely, lobes 1 and 2 were dominated by crNsd1.crNTC, and crH2-Q2.crCdh1+crNsd1.crAtm+crCasp8.crArid1a, respectively, which were also major clones in primary tumor (FIG. 7B). However, lobe 3 was dominated by crElf3.crFbxw7+crRb1.crCasp8, which were not found as major clones in primary tumor; the case of lobe 4 echoes that of lobe 3 with a more complex metastatic clonal mixture, in which most of the dominant clones (crBcor.crKdm5c, crAcvr2a.crNTC, crRb1.crCasp8, crCdkn2a.crApc, crApc.crKmt2b, crRasa1.crNf2, crElf3.crFbxw7 and crPten.crKdm5c) were not found as major clones in the primary tumor (FIG. 7B).

To quantify the metastasis-specific signature of double mutants, the number of times a crRNA array was considered as metastasis-enriched (i.e. a dominant clone in a lung lobe or extra-pulmonary metastasis (>=2% total reads) but not a dominant clone in the corresponding primary tumor of the same mouse) was calculated. Top ranked metastasis-specific dominant crRNA arrays were found to be crCic.crKmt2b, crCdkn2a.crApc, crRasa1.crNf2, crApc.crKmt2b, crNf2.crPik3r1, crNf2.crRnf43, among 23 enriched crRNA arrays, with crCic.crKmt2b being metastasis-enriched 55% (6/11 mice) of the time. These data suggest strong genetic signatures of metastasis-specific co-drivers, which have notably been difficult to parse from single-gene studies. Collectively, the results presented herein demonstrate the power of in vivo Cpf1 crRNA array screens for mapping and identification of genetic interactions in an unbiased manner.

Due to the complex nature of biological systems, a single gene is often far from sufficient to explain the biological or pathological variation observed in health and disease. Genetic interactions are the building blocks of highly connected biological networks, and their modular nature enables biological pathways to take on a variety of forms—linear, branching divergent, convergent, feed-forward, feedback, or any combination of the above. In systems biology, numerous theories and algorithms have been developed to understand such complex networks and to predict genetic interactions. However, predictions have often been surprised by unexpected experimental findings, urging for experimental testing of combinatorial perturbations in a systems manner.

High-throughput genetic screens are a powerful approach for mapping genes to their associated phenotypes. Unbiased and quantitative analysis of double knockouts enables phenotypic assessment of all possible combinations of any given gene pairs. Advances in high-throughput technologies utilizing RNA-interference-based gene knockdown or CRISPR/Cas9-based gene knockout, activation and repression, have enabled genome-scale screening in multiple species across various biological applications. While high-throughput genetic perturbation approaches have been developed to map out the landscape of genetic interactions in yeast and in worms, large-scale double knockout studies in mammalian species are scarce, due to the exponentially scaling number of possible gene combinations and the technological challenges of generating and screening double knockouts. Recently, several high-throughput double perturbations have been performed in mammalian cells using RNA interference (RNAi) or clustered regularly interspaced short palindromic repeats (CRISPR)/Cas9 technologies.

However, RNAi-based methods act on the level of mRNA silencing. Though CRISPR/Cas9-based methods can induce complete knockouts, the dependence of Cas9 on a trans-activating crRNA (tracrRNA) requires multiple sgRNA cassettes, hindering the scalability of Cas9-mediated high-dimensional screens, and making in vivo genetics more difficult.

Cpf1 was recently identified and characterized as a single-effector RNA-guided endonuclease with two orthologs from Acidaminococcus (AsCpf1) and Lachnospiraceae (LbCpf1) capable of efficient genome-editing activity in human cells. Unlike Cas9, Cpf1 requires only a single 39-42-nt crRNA without the need of an additional trans-activating crRNA, enabling one RNA polymerase III promoter to drive an array of several crRNAs targeting multiple loci simultaneously. This unique feature of the Cpf1 nuclease greatly simplifies the design, synthesis and readout of multiplexed CRISPR screens, making it a suitable system to carry out combinatorial screens.

Considering that cancer is a polygenic disease of malignant somatic cells, a Cpf1 double knockout screen was designed herein and performed in a mouse model of malignant transformation and early tumorigenesis. In this setting, successful mapping of all permutations of crRNA arrays targeting combinations of two putative non-oncogenes was demonstrated, revealing a wide array of unexpected synergistic gene pairs. The most highly connected ‘hub’ genes were epigenetic factors such as Kmt2c, Atrx, Kdm5c, Setd2, Kdm6a, and Arid1a, suggesting that the multifarious interactions of these factors, whether direct or indirect, lead to drastically accelerated tumorigenesis upon loss-of-function. Without wishing to be limited by any theory, this finding might explain why, despite being frequently mutated in human cancers, single knockouts of such factors rarely lead to tumorigenesis in vivo (though only a limited number of these genes have thus far been studied in animal models). In that sense, epigenetic modifiers might function as genetic buffers, redundant backup pathways, modifiers or amplifiers of multiple other apparently unrelated pathways. Many of the synergistic interactions identified through the screen were subsequently found to be significantly co-mutated across multiple cancer types. In a more complex biological process such as metastasis, which includes a cascade of primary tumor growth, inducing angiogenesis and lyphangiogenesis, extravasation, circulation, extravasation, colonization and immunological interactions, the screen is capable of detecting robust signatures of selection and revealing modes and patterns of clonal expansion of complex pools of double mutants in vivo. Multiplexed Cpf1 screens thus represent a powerful tool for studying genetic interactions with unparalleled simplicity and specificity.

As shown herein, multiplexed Cpf1 screens can enable the high-throughput discovery of synergistic interactions by examining patterns of crRNA array enrichment. On the flip side, crRNA array depletion screens would enable the identification of synthetically lethal gene mutations in cancer, potentially opening new avenues for therapeutic discovery (FIG. 7D). While the focus was on TSGs in the present study, CCAS screens can be easily tailored for any particular gene set in any biological context. The present study serves as a proof-of-principle with an unbiased, medium size library targeting all pairwise combinations of a selected set of genes. More comprehensive combinatorial screens are feasible through this approach simply by increasing the number and complexity of crRNA arrays in the library, as well as expanding the target cell pool and/or number of experimental animals accordingly. Considering that Cpf1 can easily target more than two loci with a single crRNA array, multiplexing 3 or more crRNAs in each array enables direct screens of triple knockouts and even higher-dimension genetic interactions in vivo.

### Example 8: High-Density In Vivo Profiling of Metastatic Double Knockouts Using Cpf1

The materials and methods employed in Experimental Example 8 are now described.

Design of the MCAP-MET library: The top 23 ranked “tumor suppressors” from the human MET500 cohort (Robinson, D. R. et al. (2017) Nature 548, 297-303) were collected, and combined with 3 top hits from a previous mouse metastasis screen (Nf2, Trim72, and Ube2g2) (Chen, S. et al. (2015) Cell 160, 1246-1260) for a final set of 26 genes. The complete exon sequences of these 26 genes were analyzed to extract all possible Cpf1 spacers (i.e., all 20 mers beginning with the Cpf1 PAM, 5′-TTTV). Each of these 20 mers was then reverse complemented and mapped to the entire mm10 reference genome by Bowtie 1.1.2, with settings -n 2 -l 18 -p 8 -a -y --best -e 90 (Langmead, et al. (2009), Genome Biol. 10, R25). After filtering out all alignments that contained mismatches in the final 3 basepairs (corresponding to the Cpf1 PAM) and disregarding any mismatches in the fourth to last basepair, the number of genome-wide alignments were quantified for each crRNA using all 0, 1, and 2 mismatch (mm) alignments. A total mismatch score (MM score) was calculated for each crRNA using the following formula: MM score=0 mm*1000+1 mm*50+2 mm*1. The number of consecutive thymidines was counted in each crRNA, using the following formulas: T score=100/(max_consecutive_Thymidines). The crRNAs were sorted corresponding to each target gene by low MM score and high T score. Finally, the top 4 crRNAs for each gene were chosen. In the event of ties, crRNAs targeting constitutive exons and/or the first exon were prioritized. 52 NTC crRNAs were randomly generated. In combination with the 104 crRNAs targeting 26 genes, a total of 5,200 DKO, 5,408 SKO, and 1,326 NTC-NTC arrays were designed for a total of 11,934 arrays (MCAP-MET library). Each gene pair is represented by 16 DKO arrays, while each single gene condition is represented by 208 SKO arrays. For SKO crRNA arrays, each gene-targeting crRNA was placed in the first position of the crRNA array and the NTC crRNAs were toggled through the second position. For each oligo, a degenerate 10 mer was appended following the U6 termination sequence to serve as a barcode for downstream clonality analysis. After pooled oligo synthesis (CustomArray), Gibson cloning was used to insert the MCAP-MET library into the BsmbI-linearized crRNA expression vector.

Cell lines: A non-small cell lung cancer (NSCLC) cell line (KPD cell line) was transduced with LentiCpf1 to generate Cpf1-positive cells (LCC-Cpf1). All cell lines were grown under standard conditions using DMEM containing 10% FBS, 1% Pen/strep in a 5% CO₂ incubator.

Lentiviral library production: The LentiCpf1 and Lenti-MCAP-MET library plasmids were used for lentiviral production. Briefly, envelope plasmid pMD2.G, packaging plasmid psPAX2, and LentiCpf1 or Lenti-MCAP-library plasmid were added at ratios of 1:1:2.5, and then polyethyleneimine (PEI) was added and mixed well by vortexing. The solution was left at room temperature for 10-20 min, and then the mixture was added dropwise into 80-90% confluent HEK293FT cells and mixed well by gently agitating the plates. Six hours post-transfection, fresh DMEM supplemented with 10% FBS and 1% Pen/Strep was added to replace the transfection media. Virus-containing supernatant was collected at 48 h and 72 h post-transfection, and was centrifuged at 1500 g for 10 min to remove the cell debris; aliquoted and stored at −80° C. Virus was titrated by infecting LCC cells at a number of different concentrations, followed by the addition of 3 μg/mL puromycin at 24 h post-infection to select the transduced cells. The virus titers were determined by calculating the ratios of surviving cells 48 or 72 h post infection and the cell count at infection.

Nextera analysis of indels generated by Cpf1: CrRNA arrays (crPten.crNf1 and crNf1.crPten) were cloned into Lenti-U6-crRNA vector, and virus was generated for transduction of KPD cell line. Pten spacer=TGCATACGCTATAGCTGCTT (SEQ ID NO: 9,709); Nf1 spacer=TAAGCATAATGATGATGCCA (SEQ ID NO: 9,710). Seven days after transduction and puromycin selection, genomic DNA was harvested from the cells in culture. The surrounding genomic regions flanking the target sites of crPten and crNf1 were first amplified by PCR using the following primers (5′-3′): Pten_fwd=ACTCACCAGTGTTTAA CATGCAGGC (SEQ ID NO: 9,711), Pten_rev=GGCAAGGTAGGTACGCATTTGCT (SEQ ID NO: 9,712); Nf1_fwd=AGCAGCTGTCCTGGCTGTTC (SEQ ID NO: 9,713), Nf1_rev=CGTGCACCTCCCTTGTCAGG (SEQ ID NO: 9,714). Nextera XT library preparation was then performed according to manufacturer protocol. Reads were mapped to the mm10 mouse genome using BWA (Li, H. & Durbin, R. (2009) Bioinforma. Oxf Engl. 25, 1754-1760), with the settings bwa mem -t 8 -w 200. Indel variants were first processed with Samtools (Li, H. et al. (2009) Bioinformatics 25, 2078-2079). with the settings samtools mpileup -B -q 10 -d 10000000000000, then piped into VarScan v2.3.9 (Koboldt, D. C. et al. (2012) Genome Res. 22, 568-576) with the settings pileup2indel --min-coverage 1 --min-reads2 1 --min-var-freq 0.00001.

Evaluation of in vivo library diversity in the absence of mutagenesis: A library of degenerate 8 mers was synthesized and cloned into the crRNA expression vector. After lentiviral production, LCC cells were transduced with the 8 mer lentiviral library and selected by puromycin. 4×10⁶ LCC-8 mer cells were subcutaneously injected both in Rag1^{-/-} and nu/nu mice. Twelve days post-transplantation, mice were sacrificed and tumors were isolated for genomic preparation and readout.

MCAP in a mouse model of metastasis: Library transduction was performed with three infection replicates at high coverage and low MOI. Briefly, according to the viral titers, MCAP-MET lentiviruses were added to a total of 1×10⁸ LCCCpf1 cells at calculated MOI of ≤0.2 and incubated 24 h before replacing the virus-containing media with 3 g/mL puromycin containing fresh media to select the virus-transduced cells. Approximately 2.5×10⁷ cells confer a -2,000× library coverage. MCAP-MET library-transduced cells were cultured under the pressure of 3 μg/mL puromycin for 7 days before injection or cryopreservation. MCAP library-transduced LCC-Cpf1 cells were injected subcutaneously into the right and left flanks of nu/nu mice at 4×10⁶ cells per flank (˜350× coverage per transplant).

Mouse tumor dissection: Mice were sacrificed by carbon dioxide asphyxiation followed by cervical dislocation. Tumors and lungs were manually dissected, then fixed in 10% formalin for 24-96 hours, and transferred into 70% Ethanol. Tissues were flash frozen with liquid nitrogen, and ground in 5 mL Frosted polyethylene vial set (2240-PEF) in a 2010 GenoGrinder machine (SPEXSamplePrep). Homogenized tissues were then used for DNA extraction.

Genomic DNA extraction: 200-800 mg of frozen ground tissue were re-suspended in 6 mL of NK Lysis Buffer (50 mM Tris, 50 mM EDTA, 1% SDS, pH 8.0) supplemented with 30 μL of 20 mg/mL Proteinase K (Qiagen) in 15 mL conical tubes, and incubated at 55° C. bath overnight. After all the tissues were lysed, 30 μL of 10 mg/mL RNAse A (Qiagen) was added, mixed well and incubated at 37° C. for 30 min. Samples were chilled on ice and then 2 mL of pre-chilled 7.5 M ammonium acetate (Sigma) was added to precipitate proteins. The samples were inverted and vortexed for 15-30s and then centrifuged at ≥4,000 g for 10 min. The supernatant was carefully decanted into a new 15 mL conical tube, followed by the addition of 6 mL 100% isopropanol (at a ratio of 0.7), inverted 30-50 times and centrifuged at ≥4,000 g for 10 minutes. At this time, genomic DNA became visible as a small white pellet. After discarding the supernatant, 6 mL of freshly prepared 70% ethanol was added, mixed well, and then centrifuged at ≥4,000 g for 10 min. The supernatant was discarded by pouring; and remaining residues was removed using a pipette. After air-drying for 10-30 min, DNA was re-suspended by adding 200-500 μL of Nuclease-Free H₂O. The genomic DNA concentration was measured using a Nanodrop (Thermo Scientific), and normalized to 1000 ng/L for the following readout PCR.

MCAP library readout: MCAP library readout was performed using a 2-step PCR approach. Briefly, in the 1st round PCR, enough genomic DNA was used as template to guarantee coverage of the library abundance and representation. For example, assuming 6.6 pg of gDNA per cell, 20-48 μg of gDNA (≥75×) was used per sample. For the 1st PCR, the sgRNA-included region was amplified using primers specific to the MCAP vector using Phusion Flash High Fidelity Master Mix (ThermoFisher) with thermocycling parameters: 98° C. for 1 min, 15 cycles of (98° C. for is, 60° C. for 5 s, 72° C. for 15s), and 72° C. for 1 min. Fwd: AATGGACTA TCATATGCTTACCGTAACTTGAAAGTATTTCG (SEQ ID NO: 9,715); Rev: CTTTAGTTT GTATGTCTGTTGCTATTATGTCTACTATTCTTTCCC (SEQ ID NO: 9,716) In the 2nd PCR, 1st round PCR products for each biological repeats were pooled, then 1-2 μL well-mixed 1st PCR products were used as the template for amplification using sample-tracking barcode primers with thermocycling conditions as 98° C. for 1 min, 15 cycles of (98° C. for is, 60° C. for 5 s, 72° C. for 15s), and 72° C. for 1 min. The 2^{nd} PCR products were quantified in 2% E-gel EX (Life Technologies) using E-Gel® Low Range Quantitative DNA Ladder (ThermoFisher), then the same amount of each barcoded samples were combined. The pooled PCR products were purified using QIAquick PCR Purification Kit and further QIAquick Gel Extraction Kit from 2% E-gel EX. The purified pooled library was quantified in a gel-based method. Diluted libraries with 5-20% PhiX were sequenced with HiSeq 4000 systems (Illumina) with 150 bp paired-end read length.

MCAP-MET plasmid library readout and analysis: Raw paired-end fastq read files were first merged to single fastq files by PEAR (Zhang, J. et al. (2014). Bioinformatics 30, 614-620). with the settings -y 8G -j 8 -v 3. The merged fastq files were then filtered and demultiplexed using Cutadapt (Martin, M. (2011) EMBnet.journal 17, 10-12), using two different sets of adapters for extraction of crRNA array sequences or the 10 mer barcode. For the crRNA array, the following settings were used: cutadapt --discard-untrimmed -g tcttGTGGAAAGGACGAAACACCg (SEQ ID NO: 9,731), followed by cutadapt --discard-untrimmed -a TGTAGATTTTTTT (SEQ ID NO: 9,758). The trimmed sequences were then mapped to the MCAP-MET library using Bowtie (Langmead, et al. (2009), Genome Biol. 10, R25): bowtie -v 3 -k 1 -m 1. For the Omer barcodes, we used the following Cutadapt settings: cutadapt --discard-untrimmed -a aagcttggcgtGGATC (SEQ ID NO: 9,759), followed by cutadapt --discard-untrimmed -g TACTAAGTGTAGATTTTTTT (SEQ ID NO: 9,760). The resultant sequences were quantified to a reference of all possible 10 mer sequences. Reads that successfully mapped to both the MCAP-MET library and contained a valid barcode were tabulated.

Processing of MCAP-MET crRNA array abundance in cells and tumors: PEAR-merged fastq files were filtered and demultiplexed using Cutadapt. To remove extra sequences downstream (i.e. 3′ end) of the crRNA array sequences, including the DR and U6 terminator, the following settings were used: cutadapt --discard-untrimmed -e 0.1 -a aagcttggcgtGGATCCGATATCa (SEQ ID NO: 9,761) -m 80. As the forward PCR primers used to readout crRNA array representation were designed to have a variety of barcodes to facilitate multiplexed sequencing, these filtered reads were then demultiplexed with the following settings: cutadapt -g file:fbc.fasta --no-trim, where fbc.fasta contained the 12 possible barcode sequences within the forward primers. Finally, to remove extraneous sequences upstream (i.e. 5′ end) of the crRNA array spacers, the following settings were used: cutadapt --discard-untrimmed -e 0.1 -g tcttGTGGAAAGGACGAAACACCg (SEQ ID NO: 9,731) -m 80. The 5′ DR were removed as follows: cutadapt --discard-untrimmed -e 0.1 -g TAATTTCTACTAAGTGTAGAT (SEQ ID NO: 21,696) -m 80. The filtered fastq reads were then mapped to the MCAP-MET reference index. To do so, a Bowtie index of the MCAP-MET library was generated using the bowtie-build command in Bowtie 1.1.2 (Langmead, et al. (2009), Genome Biol. 10, R25). Using these bowtie indexes, the filtered fastq read files were mapped using the following settings: bowtie -n 2 -k 1 -m 1 --best. These settings ensured only single-match reads would be retained for downstream analysis. For data processing on the level of barcoded-crRNAs, the same trimmed fastq files as above were utilized, but instead the barcoded-crRNA plasmid library was used as the reference index.

Analysis of MCAP crRNA array library representation: Using the resultant mapping output, the number of reads that had mapped to each crRNA array within the library were quantified. The number of reads in each sample was normalized by converting raw crRNA array counts to reads per million (rpm). The rpm values were then subject to log₂ transformation for certain analyses. To generate Spearman correlation heat maps, the NMF R package was used. Where applicable, linear regression lines and 95% confidence intervals were calculated. For comparing cells, primary tumors, and lung metastases, crRNA array abundances were averaged within sample groups and linear regression was performed using the NTC-NTC arrays as a model for neutral selection. Significant outliers were identified using the outlierTest function from the car R package. For gene/gene pair analyses, the corresponding SKO and DKO arrays were first averaged together, then aggregated by sample type. Linear regression was performed using all SKO/DKO genotypes, and outliers were identified as above.

Clone-level analysis of MCAP-MET samples: The data were analyzed at the clone level using the barcoded-crRNA abundances. The counts in each sample were first converted to percentages of total reads. Two different frequency cutoffs were used for considering clones: ≥0.01% and ≥0.001%. Differences in the number of clones between sample types was assessed by Wilcoxon rank sum test, and visualized after log₂ transform. Empirical CDFs were calculated after combining all the clones in a given sample group; statistical differences in clone size distributions was assessed by Kolmogorov-Smirnov test. The Shannon diversity index was also calculated on each sample with the vegan R package; statistical differences were assessed by Wilcoxon rank sum test.

Enrichment analysis of MCAP-MET genotypes: To identify crRNA arrays that were enriched in individual samples, the 1,326 NTC-NTC arrays were utilized for modeling the empirical null distribution. Enriched crRNA arrays were subsequently called at FDR<0.5%. These results were aggregated to the single gene/gene pair level, then tabulated across samples. Finally, all of the significant crRNA arrays associated with each genotype were counted.

Identification of synergistic mutation combinations: The synergy coefficient (SynCo) for each gene pair was defined with the following formula: SynCo=DKO_{NM}−SKO_{N}−SKO_{M}. The DKO_{NM} value is the average log₂ rpm abundance of all corresponding DKO crRNA arrays (i.e., crN.crM), while SKO_{N} and SKO_{M} values are defined as the average log₂ rpm abundance of all corresponding SKO crRNA arrays. By this definition, a SynCo score>0 would indicate that a given DKO crRNA array is synergistic, as the DKO score would thus be greater than the sum of the individual SKO scores. The SynCo of each gene pair was calculated and it was assessed whether the DKO abundances were statistically significantly higher than both SKO abundances by Wilcoxon rank sum test.

To generate a library-wide map of the relative selective advantages for each gene pair vs. single gene knockout, the aggregated gene-level abundances were utilized in lung metastasis samples. The abundance of each DKO was compared to its reference SKO, and the data visualized in a heat map. Each column refers to the reference SKO, while each row denotes the modulatory effects of the second KO.

Statistics: All statistical tests are two-sided.

Blinding statement: Investigators were not blinded for sequencing data analysis, tumor engraftment, or organ dissection.

The results of the experiments from Example 8 are now described.

Metastasis is the major lethal factor of solid cancers. However, the complex genetic interactions underlying the metastatic phenotype of tumor cells have remained elusive. A streamlined approach for constructing global maps of metastasis gene networks is key to understanding metastasis at the systems level. Herein was developed MCAP (Massively-parallel crRNA array profiling), an approach for high-throughput interrogation of genetic combinations in vivo. A UMI-barcoded, high-density, high-redundancy MCAP library was designed with 11,934 crRNA arrays targeting 325 pairwise combinations of genes significantly mutated in human metastases, and the metastatic potential of all combinations were functionally interrogated in parallel in mice. Enrichment, synergy and clonality analyses unveiled a quantitative landscape of genetic interactions in metastasis.

Metastasis, the major lethal factor of solid tumors, is controlled by a complex network of genetic interactions. However, a systems-level understanding of the genetic interactions driving metastatic spread is lacking. Due to various technological challenges, high-throughput in vivo interrogation of double knockouts in mammalian species has not yet been reported in the literature. Thus, a streamlined approach is essential for rapidly mapping out a global, clinically relevant metastasis gene networks with high resolution.

The discovery and characterization of the type V CRISPR system Cpf1 (CRISPR from Prevotella and Francisella, also known as Cas12a) has empowered genome editing of multiple loci in individual cells. Cpf1 is a single component RNA-guided nuclease that can mediate target cleavage with a single crRNA. Unlike Cas9, Cpf1 does not require a tracrRNA, which greatly simplifies multiplexed genome editing of two or more loci simultaneously through the use of a single crRNA array targeting different genes. Thus, Cpf1 is an ideal system for investigating genetic interactions in vivo, with substantial advantages in library design and readout when compared to Cas9-based approaches. Leveraging the Cpf1 system, MCAP (Massively-parallel crRNA array profiling) was developed: an approach for in vivo high-throughput quantitative mapping of double or higher dimensional genetic perturbations. A UMI-barcoded high-density MCAP library was designed with 11,934 crRNA arrays (SEQ ID NOs: 9,762-21,695) targeting 325 gene pairs significantly mutated in human metastases, with high-redundancy crRNA array coverage for each gene and gene pair. Using this library, MCAP was demonstrated to be a powerful tool for functional interrogation of hundreds of double knockouts and their single knockout counterparts for their metastatic potential in mice.

To establish a CRISPR/Cpf1 lentiviral system for characterization of mutation combinations in cancer, a human-codon-optimized LbCpf1 expression vector (pLenti-EFS-Cpf1-blast, LentiCpf1 for short) and a crRNA expression vector (pLenti-U6-DR-crRNA-puro, Lenti-U6-crRNA for short) were generated (FIG. 1A). In order to facilitate direct and targeted double knockout studies using a single crRNA array, oligos were designed with a 5′ homology arm to the base vector, followed by a crRNA, the direct repeat (DR) sequence for Cpf1, a second crRNA, a U6 terminator, and finally a 3′ homology arm (cr1-DR-cr2). As the oligos each contain two crRNAs, these constructs were termed crRNA arrays. Linearization of the Lenti-U6-crRNA vector enables one-step cloning of the crRNA array into the vector by Gibson assembly, producing the double knockout crRNA array expression vector (pLenti-U6-DR-cr1-DR-cr2-puro) (FIG. 1B). These constructs were first tested for their ability to induce double knockouts in a murine cancer cell line (KPD) in vitro. After infection with LentiCpf1 to generate Cpf1⁺ KPD cells, they were transduced with lentiviruses carrying a crRNA array targeting Pten and Nf1 (FIG. 8A). To confirm whether Cpf1 can mediate mutagenesis regardless of the position of each crRNA within the array, two permutations of the Pten and Nf1 crRNA array (crPten.crNf1 and crNf1.crPten, all with 20 nt spacers), were generated. Both crPten.crNf1 and crNf1.crPten crRNA arrays generated indels at both loci in Cpf1+ KPD cells (FIG. 8B). These data confirmed the ability of single crRNA arrays with Cpf1 to generate double knockouts in mammalian cells.

In order to perform high-throughput genetic investigation of metastasis suppression in vivo, it is important to evaluate the library diversity that can be accommodated upon introduction of the cell pool. To that end, a mock library of degenerate 8 mers was constructed and cloned into the base Lenti-U6-crRNA vector (FIG. 26A). After production of lentivirus, KPD cells were transduced and 4×10⁶ 8 mer-barcoded cells were transplanted into nufnu (n=2) or Rag1^{-/-} mice (n=4). The resultant small nodules from the injection site were harvested 12 days later, and the barcodes were deep sequenced. Out of the 48=65,536 possible 8 mers, nearly 100% of them were recovered in vivo, with an average of 65,534.5/65,536 (99.99%) 8 mers identified in nunu mice, and 64,500.75/65,536 (98.42%) recovered in Rag1^{-/-} mice (FIG. 26B). Their respective abundances followed a log-normal distribution (FIG. 26C), indicating adequate coverage of the degenerate 8 mer library in vivo in the absence of mutagenesis. It was concluded that the in vivo transplant model is sufficiently powered for high-throughput interrogation of metastasis drivers, using libraries containing at least up to 65,536 unique oligos.

To investigate whether Cpf1 multiplexed gene targeting could be utilized for high-throughput investigation of mutation combinations, massively-parallel Cpf1-crRNA array profiling (MCAP) was developed. Considering the resolution of library complexity under in vivo cellular dynamics, genes significantly mutated in a human metastasis cohort (MET-500) (Robinson, D. R. et al. (2017) Nature 548, 297-303), and the top hits from a single-gene metastasis screen in mice (Chen, S. et al. (2015) Cell 160, 1246-1260) were focused on (FIG. 27A). For these 26 metastasis driver candidates (Trp53, Cdkn2a, Pten, Rb1, Brca2, Atm, Kmt2c, Apc, Kmt2d, Arid1a, Nf1, Zfhx3, Fanca, Wrn, Pole, Ercc5, Notch1, Chd1, Atrx, Jak1, Crebbp, Kdm6a, Arid1b, Nf2, Trim72, Ube2g2), all possible Cpf1 spacer sequences with a PAM sequence of TTTV were identified, subsequently choosing 4 crRNAs for each gene. The selection of crRNAs was based on two criteria: 1) high genome-wide mapping specificity, and 2) a low number of consecutive thymidines, since long stretches of thymidines will terminate U6 transcription. Compiling these 104 gene-targeting crRNAs and 52 additional non-targeting control (NTC) crRNAs, a metastasis-focused MCAP library (MCAP-MET) was designed composed of 1,326 NTC-NTC control arrays, 5,408 single-knockout (SKO) arrays, and 5,200 double-knockout (DKO) arrays, for a total of 11,934 arrays (FIG. 27A, SEQ ID NOs: 9,762-21,695). In the MCAP-MET library, each gene pair double knockout is represented by 16 independent DKO crRNA arrays, while each individual gene knockout is represented by 208 independent SKO crRNA arrays. In addition, a degenerate 10 mer barcode was appended after the U6 terminator sequence for downstream analysis of clonality. After pooled oligo synthesis, the MCAP-MET library was cloned into the base crRNA expression vector, and the plasmid crRNA array representation was subsequently readout by deep-sequencing the crRNA expression cassette. All 11,934/11,934 (100%) of the designed crRNA arrays were successfully cloned and were represented in a log-normal distribution (FIG. 27C). Analysis of the 10 mer barcodes revealed a normal distribution for the number of distinct barcodes associated with each crRNA array (unique barcoded-crRNAs recovered, n=774,295) (FIG. 27D). The abundances of the barcoded-crRNAs within the MCAP-MET library were also evenly distributed (FIG. 28). Thus, a barcoded MCAP library was designed and generated for targeted single and double mutagenesis of relevant metastatic candidate genes and gene pairs with high redundancy of independent targeting constructs.

Lentiviral pools were generated from the MCAP-MET plasmid library and Cpf1⁺ KPD cells were infected (FIG. 27B). One and two weeks after lentiviral transduction and antibiotic selection, the crRNA expression cassette was sequenced. High correlation to the initial plasmid library was found at both time points (FIG. 27E, FIG. 29A). Having established the successful introduction of the barcoded MCAP-MET library into Cpf1⁺ KPD cells, the metastatic potential of all these 11,934 crRNA arrays targeting 325 mutation combinations and 26 single mutations were quantitatively mapped in vivo. The MCAP-MET cell pool was injected (4×10⁶ cells per mouse, ˜350× coverage) subcutaneously into nu/nu mice (n=10). After 6 weeks, the primary tumors (n=10) and lung lobes (n=37) were collected, and crRNA array sequencing was performed as before (FIG. 27B). The data from the level of barcoded-crRNAs were first analyzed, in order to assess the dynamics of selection in the metastasis model. The number of “clones” (approximated by barcoded-crRNAs) that surpassed 0.001% of the total tumor burden by barcoded-crRNA abundance were quantified (FIG. 30A). Clear evidence of progressive selection was found as the in vitro cell pools formed primary tumors and lung metastases (FIG. 30B). The cell pools had significantly more unique clones represented at ≥0.001% frequency than primary tumors (Wilcoxon rank sum test, p=0.0002) and lung metastases (p=0.0001), as did primary tumors compared to lung metastases (p=0.0162). This result was consistent at an alternate cutoff of ≥0.01% frequency (FIGS. 31A-31B).

The relative abundances of these various barcoded-crRNA clones were examined (FIG. 30C). Primary tumors and lung metastases, but not cell pools, were dominated by a handful of clones. The empirical cumulative density function of all represented clones in cells, primary tumors, and lung metastases was calculated (FIG. 30D). This analysis demonstrated that lung metastases are more skewed towards higher % frequencies per clone than primary tumors (Kolmogorov-Smirnov test, p<2.2*10-6), though both populations are significantly more skewed than the cell pool. As an alternative measure, the Shannon diversity index was calculated for each sample. The clonal abundances of the cell pools were significantly more diverse than primary tumors or lung metastases (Wilcoxon rank sum test, p=0.0002 and p=3.28*10-7), while primary tumors were in turn more diverse than lung metastases (p=0.0212) (FIG. 31F). These results were consistent at a higher cutoff of ≥0.01% frequency (FIG. 31C-31E). Collectively, the clone-level analyses illustrated the progressive selection pressures on the cells as they formed primary tumors and metastasized to the lung.

To map the metastatic potential of all these single and double knockouts in an unbiased manner, the barcoded-crRNA counts were collapsed to the crRNA array level (Supplementary FIG. 29B). Utilizing the 1,326 NTC-NTC crRNA arrays as an empirical null distribution, crRNA arrays enriched at false discovery rate (FDR)<0.5% were identified in each sample. Within primary tumors, 24 single genes and 23 gene pairs were consistently enriched in ≥50% of samples. Top single genes included Fanca, Jak1, and Nf2, while top gene pairs included Nf_Arid1b, Nf2_Pten, Nf2_Apc, Nf2_Chd1, and Kmt2d_Chd1. Within lung metastases, 23 single genes and 25 gene pairs were enriched across ≥50% of samples. Top single genes in lung metastases included Nf2, Apc, and Jak, while the top gene pairs were Nf2_Chd1, Nf2_Arid1b, and Nf2_Trim72. Intersecting the DKO lists, 5 gene pairs were enriched in half of primary tumors but not in lung metastases, while 7 gene pairs were enriched in half of lung metastases but not in half of primary tumors (FIG. 30E). Note that each single gene is represented by 208 independent SKO arrays in the MCAP-MET library whereas each gene pair has 16 DKO arrays, to account for this difference, the percentage of arrays that were called as enriched in at least one lung metastasis sample were tabulated, for each single gene and gene pair (FIGS. 30F-30I). This analysis revealed that no single genes were found to have more than 40% of their SKO arrays enriched in lung metastases, with the most consistent performer being Nf2 at 32.21% (FIGS. 30H-30I). For example, 10 independent crRNA arrays out of 16 were enriched in at least one lung metastasis for Nf2_Rb1 double knockout (FIG. 2j), with 9/16 arrays for Nf2_Pten (FIG. 30K) and Nf2_Trim72 (FIG. 30L). In total, 9 gene pairs had ≥43.75% (7/16) of their DKO arrays enriched in a lung metastasis sample. These were Nf2_Rb1, Nf2_Pten, Nf2_Trim72, Nf2_Apc, Nf2_Arid1b, Nf2_Chd1, Nf2_Jak, Nf2_Nf1, and Notch1_Apc.

In addition to the binary FDR-based enrichment analysis above, the relative metastatic potential of the various genotypes represented in the MCAP-MET library were quantitatively compared using the information of relative abundance for all crRNA arrays in each sample. Aggregating by sample type, the average abundances of each crRNA array in cell pools (n=6), primary tumors (n=10), and lung metastases (n=37) were compared (FIG. 32A, FIG. 32C, FIG. 32E). To obtain a reference of neutrality, the 1,326 NTC-NTC arrays were used to calculate the linear regression between different sample types, as the Spearman correlations of NTC-NTC array average abundance between sample types are highly significant (e.g. FIG. 27E, FIG. 32A, FIG. 32C; p<2.2*10⁻¹⁶ for all comparisons). By comparison to the NTC-NTC linear regression, strong selection between cells and primary tumors was seen (FIG. 32A), as well as cells and lung metastases (FIG. 32C), as evidenced by the existence of outliers. Looking to identify the specific single or double knockouts exhibiting strong selection in vivo, the constituent crRNA arrays for each SKO or DKO genotype were averaged on a sample-by-sample basis, then the data were aggregated by sample type. In order to pinpoint the genotypes with the strongest selective advantage out of the entire MCAP-MET library, for the gene-level analyses all targeting genes/pairs were used for linear regression modeling. The top gene pairs that were significantly favored in primary tumors relative to cell pools (outlier test, p<0.05) included Nf2_Trim72, Nf2_Chd1, Nf2_Arid1b, Nf2_Kdm6a, Kmt2d_Chd1, and Nf2_Rb (FIG. 32B). Nf2 was the only single gene found to be significantly selected for in tumors relative to cells. A similar set of gene pairs were enriched in lung metastases compared to cell pools, with a notable exception of Jak1_Kmt2c, which was not significantly enriched in primary tumors vs. cell pools (FIG. 32D). Primary tumors were directly compared to lung metastases (FIG. 32E), and specific gene pairs were identified with evidence of significant negative or positive selection relative to the entire MCAP-MET library (p<0.05) in lung metastases relative to primary tumors. From the overall library-wide regression, while 18 double knockouts were found to be outliers in metastasis-primary tumor regression, no single knockouts were found to be significantly favored in metastasis over primary tumor. Positively selected mutation combinations in lung metastases included Nf2_Trim72, Nf2_Chd1, Kmt2d_Chd1, Jak1_Kmt2c, Ube2g2_Apc, Kmt2d_Rb1, Nf1_Pten, and Cdkn2a_Rb1. On the other hand, genotypes that were relatively depleted in lung metastases included Nf2_Cdkn2a, Ube2g2_Arid1b, Nf2_Crebbp, Ube2g2_Cdkn2a, Ube2g2_Nf2, and Cdkn2a_Wrn.

Analyses suggested that certain gene pairs may be especially synergistic in promoting tumorigenesis and/or metastasis. To quantitatively identify such mutation combinations, the gene-level data were utilized to compare the normalized abundances of each DKO gene pair with its two constituent SKO genes across all primary tumors and lung metastases (total n=47) (FIG. 33A). Gene pairs were first identified that were significantly more abundant than their respective single gene counterparts (Wilcoxon rank sum test, p<0.05). Since the effects of a mutational combination may simply be additive rather than truly synergistic, a synergistic coefficient (SynCo=DKO_{NM}−SKO_{N}−SKO_{M}) was also calculated for each gene pair (FIG. 33A). Collectively, 6 DKO genotypes were found that were significantly more abundant than the two corresponding SKO genotypes and with a SynCo>0 (FIGS. 33B-33C). The synergistic gene pairs identified were Nf2_Trim72, Chd1_Nf2, Chd1_Kmt2d, Jak1_Kmt2c, Kmt2d_Pten, and Nf1_Pten (FIGS. 33D-33I). Of note, 5/6 of these gene pairs were found to be among the positively selected genotypes in lung metastases vs. primary tumors (FIG. 33F). Finally, a library-wide map of the selective advantage of each gene pair DKO was constructed relative to the corresponding single gene SKO (FIG. 34). Collectively, these data point to specific mutation combinations with heightened metastatic potential in vivo, and highlight the power of MCAP for high-throughput interrogation of genetic interactions in complex biological systems.

Due to the complex nature of biological systems, a single gene is far from sufficient to explain the clinical and pathological variation observed across patients. Genetic interactions are the building blocks of highly connected biological networks, and their modular nature enables biological pathways to take on a variety of forms—linear, branching divergent, convergent, feed-forward, feedback, or any combination of the above. These complex interactions may account for a substantial part of variation for intricate phenotypes in complex biological or pathological processes such as cancer. Numerous theories and algorithms have been developed to understand such complex networks and to predict genetic interactions. However, predictions have often been surprised by unexpected experimental findings, urging for experimental testing of combinatorial perturbations in a systems manner.

High-throughput genetic studies are a powerful approach for mapping genes to their associated phenotypes. Unbiased and quantitative analysis of double knockouts enables phenotypic assessment of all possible combinations of any given gene pairs. Advances in high-throughput technologies utilizing RNA-interference-based gene knockdown or CRISPR/Cas9-based gene knockout, activation and repression, have enabled genome-scale studies in multiple species across various biological applications. While high-throughput genetic perturbation approaches have been developed to map out the landscape of genetic interactions in yeast and in worms, large-scale double knockout studies in mammalian species are relatively scarce, due to the exponentially scaling number of possible gene combinations and the technological challenges of generating and evaluating double knockouts. Recently, several high-throughput double perturbations have been performed in mammalian cells using RNA interference (RNAi) or clustered regularly interspaced short palindromic repeats (CRISPR)/Cas9 technologies. However, RNAi-based methods act on the level of mRNA silencing. Though CRISPR/Cas9-based methods can induce complete knockouts, the dependence of Cas9 on a trans-activating crRNA (tracrRNA) predicates the need for multiple sgRNA cassettes when performing combinatorial knockouts, hindering the scalability of Cas9-mediated high-dimensional studies to in vivo settings.

Cpf1 is a single-effector RNA-guided endonuclease with two orthologs from Acidaminococcus (AsCpf1) and Lachnospiraceae (LbCpf1) capable of efficient genome-editing activity in human cells. Unlike Cas9, Cpf1 requires only a single 39-42-nt crRNA without the need of an additional trans-activating crRNA, enabling one RNA polymerase III promoter to drive an array of several crRNAs targeting multiple loci simultaneously. This unique feature of the Cpf1 nuclease greatly simplifies the design, synthesis and readout of multiplexed CRISPR studies, making it a suitable system to investigate mutation combinations.

In summary, the present study demonstrates the utility of MCAP for simultaneous, massively parallel profiling of single and double knockouts, implementing a high-density library design with 16 independent constructs per double knockout and 208 per single knockout. Even in a complex biological process such as metastasis, MCAP is capable of detecting robust signatures of selection in vivo and quantitatively profiling single and double mutants of strong, moderate and weak phenotypes. MCAP thus represents a powerful new tool for mapping genetic interactions in mammalian species in vivo with unparalleled simplicity and throughput.

### Example 9: Cpf1-Flip: A Flexible Sequential Mutagenesis System by Inducible crRNA Array Inversion

The materials and methods employed in Experimental Example 9 are now described.

FlipArray design and construction: The empty EFS-Cpf1-Puro; U6-FipArray vector was constructed by modification of the pY109 lentiviral vector (Zetsche, B. et al. (2017) Nat. Biotechnol. 35, 31-34). After BsmbI digestion (FastDigest Esp3I, ThermoScientific) to linearize the U6 crRNA expression cassette, oligo cloning was performed to insert a lox66 sequence, a DR, two BsmbI sites, and an inverted lox71. The empty vector thus expresses LbCpf1 and puromycin resistance from an EFS promoter, while a U6 promoter drives expression of a lox66/lox71 flanked crRNA expression module containing two BsmbI sites. BsmbI digestion and oligo cloning was then used to insert FlipArrays into the empty vector. For a given pair of crRNAs, the following oligo overhangs were used for cloning: Oligo1 5′ overhang: TAGAT; Oligo1 3′ overhang: A; Oligo2 5′ overhang: GTTAT; Oligo2 3′ overhang: A

The main body of the FlipArray was structured as such: 5′-crRNA 1-6×T -6×A-Rev.Complement(crRNA 2)-Rev.Complement(DR)-3′ In certain embodiments, the vector comprising the FlipArray comprises SEQ ID NO: 21,697.

In this study, the following oligo sequences were used to target Nf1 and Pten:

crNf1 spacer:
(SEQ ID NO: 9,710)
TAAGCATAATGATGATGCCA
crPten spacer:
(SEQ ID NO: 9,709)
TGCATACGCTATAGCTGCTT
NPF oligo 1 (to clone into vector):
(SEQ ID NO: 9,719)
TAGATTAAGCATAATGATGATGCCATTTTTTA
AAAAAAAGCAGCTATAGCGTATGCAATCTACAC
TTAGTAGAAATTAA
NPF oligo 2 (to clone into vector):
(SEQ ID NO: 9,720)
GTTATTAATTTCTACTAAGTGTAGATTGCATA
CGCTATAGCTGCTTTTTTTTAAAAAATGGCATC
ATCATTATGCTTAA

The following crRNA spacer sequences were also used, with analogous oligo designs for cloning into the Cpf1-Flip vector:

crDNMT1:
(SEQ ID NO: 9,721)
CTGATGGTCCATGTCTGTTA
crVEGFA:
(SEQ ID NO: 9,722)
CTAGGAATATTGAAGGGGGC
crFasl:
(SEQ ID NO: 9,723)
GTCCGGCCCTCTAGGCCCAC
crIdo1:
(SEQ ID NO: 9,724)
CTACAGGGAATGCACAGATG
crJak2:
(SEQ ID NO: 9,725)
ACATACATCGAGAAGAGTAA
crLgals9:
(SEQ ID NO: 9,726)
TGCAGTACCAACACCGCGTA
crB2m:
(SEQ ID NO: 9,727)
TGCACGCAGAAAGAAATAGC
crCd274:
(SEQ ID NO: 9,728)
TAAAGCACGTACTCACCGAG

Lenti-Cre vector design and construction: The Lenti-Cre vector was designed to express the Cre recombinase under a constitutive EFS promoter. The plasmid was generated by PCR amplification of Cre and EFS fragments followed by Gibson assembly into a previous lentiviral vector backbone (lentiGuidePuro) Sanjana, et al. (2014) Nat. Methods 11, 783-784).

Cell culture and genomic DNA extraction: KPD cells, E0771 cells, and HEK293T cells were cultured in DMEM supplemented with 10% FBS and 1% penicillin/streptomycin. Experiments were conducted with at least 2 independent cellular replicates. For genomic DNA extraction, approximately 500,000 cells were isolated. Cells were spun down at 500 rpm for 5 minutes and washed once with 1×PBS. After removing the supernatant, cell pellets were resuspended in 500 ul QuickExtract DNA Extraction Solution (Epicentre). Cells were then incubated at 65° C. for 20 minutes, followed by incubation at 85° C. for 5 minutes to deactivate the enzymes.

Detection of FlipArray inversion at the genomic DNA level by PCR: The following primers were used to amplify the U6 cassette from genomic DNA:

RdF:
(SEQ ID NO: 9,729)
GAGGGCCTATTTCCCATGATTCCTTCATATTT
RdR:
(SEQ ID NO: 9,730)
ACAGTGCAGGGGAAAGAATAGTAGA

PCR conditions: 98° C. 2 minutes, 32 cycles of (98° C. 1 second, 62° C. 5 seconds, 72° C. 15 seconds), 72° C. 2 minutes, 4° C. hold.

Following Qiagen PCR purification, 2 ng of the first PCR were used for the second inversion-specific or non-inverted-specific PCR. The following primers were used for detection of non-inverted or inverted FlipArrays:

NPF_F:
(SEQ ID NO: 9,731)
TCTTGTGGAAAGGACGAAACACCG
NPF_R:
(SEQ ID NO: 9,732)
TGCATACGCTATAGCTGCTTTTTTTTAAAAAATGGCA
NPF_R_inv:
(SEQ ID NO: 9,733)
TAAGCATAATGATGATGCCATTTTTTAAAAAAAAGCAG
DVF_F:
(SEQ ID NO: 9,731)
TCTTGTGGAAAGGACGAAACACCG
DVF_R:
(SEQ ID NO: 9,734)
GGGCTTTTTTAAAAAATAACAGACATGGACCATCAG
DVF_R_inv:
(SEQ ID NO: 9,735)
CTGATGGTCCATGTCTGTTATTTTTTAAAAAAGCCC

PCR conditions: 98° C. 2 minutes, 14 cycles of (98° C. 1 second, 62° C. 5 seconds, 72° C. 2 seconds), 72° C. 2 minutes, 4° C. hold. PCR reactions specific to non-inverted and inverted FlipArrays were performed and analyzed simultaneously for each sample. Quantification was done on 2% E-gel using low-range quantitative ladder (ThermoFisher), and was normalized to the first PCR product.

Detection and quantification of FlipArray inversion at the RNA transcript level: KPD cells were cultured in DMEM supplemented with 10% FBS and 1% penicillin/streptomycin. For RNA extraction, approximately 200,000 cells were isolated and spun down at 500 rpm for 5 minutes. After a PBS wash, cells were resuspended in 450 ul TRIzol. 100 ul of chloroform was then added to each tube, followed by rigorous vortexing for 15 seconds and centrifuging at 14,000 rpm for 10 minutes. The supernatant containing RNA was then purified using a Qiagen RNeasy Kit following the RNA cleanup protocol. cDNA was generated by reverse transcription with random hexamers. PCR detection of inverted crRNA FlipArray transcripts was done using the following primers:

Inv_FlipArray_F:
(SEQ ID NO: 9,736)
TGTAGATAGCGCTATAACTTCGTATAGC
Inv_FlipArray_R:
(SEQ ID NO: 9,737)
AAGCAGCTATAGCGTATGCAATC

PCR conditions: 98° C. 2 minutes, 34 cycles of (98° C. 1 second, 56° C. 5 seconds, 72° C. 5 seconds), 72° C. 2 minutes, 4° C. hold.

As a normalization control, PCR detection of Cpf1 transcripts was done using the following primers:

Cpf1_F:
(SEQ ID NO: 9,738)
TTCTTTGGCGAGGGCAAGGAGACAA
Cpf1_R:
(SEQ ID NO: 9,739)
GCACGCGCACCTCTGTATTGATCTT

PCR conditions: 98° C. 2 minutes, 40 cycles of (98° C. 1 second, 56° C. 5 seconds, 72° C. 20 seconds), 72° C. 2 minutes, 4° C. hold. Quantification of inverted FlipArray RNA abundance was done on 2% E-gel using low-range quantitative ladder (ThermoFisher), and was normalized to Cpf1 mRNA transcript abundance.

Detection of Cpf1 mutagenesis: The genomic regions flanking the crRNA target sites were amplified from genomic DNA using the following primers:

Nf1_F:
(SEQ ID NO: 9,740)
GGGTCCGATTGCCAGTACCC
Nf1_R:
(SEQ ID NO: 9,741)
AACGTGCACCTCCCTTGTCA
Pten_F:
(SEQ ID NO: 9,711)
ACTCACCAGTGTTTAACATGCAGGC
Pten_R:
(SEQ ID NO: 9,712)
GGCAAGGTAGGTACGCATTTGCT
DNMT1_F:
(SEQ ID NO: 9,742)
CTGGGACTCAGGCGGGTCAC
DNMT1_R:
(SEQ ID NO: 9,743)
CCTCACACAACAGCTTCATGTCAGC
VEGFA_F:
(SEQ ID NO: 9,744)
CTCAGCTCCACAAACTTGGTGCC
VEGFA_R:
(SEQ ID NO: 9,745)
AGCCCGCCGCAATGAAGG
Cd274_F:
(SEQ ID NO: 9,746)
GAATGGTCCCCAAGACAAAGAAGAAGA
Cd274_R:
(SEQ ID NO: 9,747)
ATTCCCAAAGGAGAACCTGTAATGAGC
Ido1_F:
(SEQ ID NO: 9,748)
TTCATTGTTCTTCACCCCATGATTGGT
Ido1_R:
(SEQ ID NO: 9,749)
CCCATGACTTTCCTAAGGAGTGTGAAA
B2m_F:
(SEQ ID NO: 9,750)
TGTCAGGTGGAGTCTAGTGGTAGAAAA
B2m_R:
(SEQ ID NO: 9,751)
ATTGGGCACAGTGACAGACTTCAATTA
Fasl_F:
(SEQ ID NO: 9,752)
CGCCTGATTCTCCAACTCTAAAGAGAC
Fasl_R:
(SEQ ID NO: 9,753)
GCAAAGAGAAGAGAACAGGAGAAAGGT
Jak2_F:
(SEQ ID NO: 9,754)
AGATTCATAGCTGTCGTTCATCACTGG
Jak2_R:
(SEQ ID NO: 9,755)
GTTAGTTCTCTTTCTGCTTCTCTGCCA
Lgals9_F:
(SEQ ID NO: 9,756)
TTTGGCATCTTCACCAAGGTAGATTGT
Lgals9_R:
(SEQ ID NO: 9,757)
TAAGCCTGGACTAAGTAAGTGAATGCC

PCR conditions: 98° C. 2 minutes, 32 cycles of (98° C. 1 second, 63° C. 5 seconds, 72° C. 20 seconds), 72° C. 2 minutes, 4° C. hold.

The genomic DNA from approximately 1000 cells was used for PCR with the NPF and DVF FlipArrays. For the TSG-Immune FlipArray library experiments, genomic DNA from approximately 6000 cells were used to account for the pooled nature of the experiment. The resultant PCR products were used for Nextera library preparation following manufacturer protocols. Reads were mapped to the mm10 or hg38 genome using BWA-MEM (Li, H ArXiv13033997 Q-Bio (2013)), with settings -t 8 -w 200. After identification of indel variants using the pileup2indel function in VarScan v2.3.9, a 1% variant frequency threshold was to identify high confidence variants for NPF and DVF experiments. A less stringent 0.2% variant frequency threshold was used for the TSG-Immune experiments due to their pooled nature.

Sample size determination: No specific methods were used to predetermine sample size.

Blinding statement: Investigators were blinded for sequencing data analysis with generic sample IDs, but not blinded for PCR or RT-PCR.

The results of the experiments from Example 9 are now described.

Mutations and genetic alterations are often sequentially acquired in various biological and pathological processes, such as development, evolution, and cancer. Certain phenotypes only manifest with precise temporal sequences of genetic events. While multiple approaches have been developed to model the effects of mutations in tumorigenesis, few recapitulate the stepwise nature of cancer evolution. A flexible sequential mutagenesis system, Cpf1-Flip, with inducible inversion of a single crRNA array (FlipArray), was created, and its application in stepwise mutagenesis in murine and human cells was demonstrated. As a proof-of-concept, Cpf1-Flip was further utilized in a pooled-library approach to model the acquisition of diverse resistance mutations to cancer immunotherapy. Cpf1-Flip offers a simple, versatile and controlled approach for precise mutagenesis of multiple loci in a sequential manner.

When loxP sites are arranged such that they point towards each other, Cre recombination leads to inversion of the intervening sequence. However, this process leads to the complete regeneration of the loxP sites, thereby allowing Cre to continually catalyze DNA inversion. As continuous Cre-mediated inversion would be counterproductive in many applications, mutant loxP sites have been characterized that enable unidirectional Cre inversion. When the mutant loxP sites lox66 and lox71 are recombined, they generate a wildtype loxP site and a double-mutant lox72. Cre has a substantially lower affinity for lox72, thus leading to mostly irreversible inversion of the floxed DNA segment.

A U6 expression cassette was designed containing two inverted BsmbI restriction sites, flanked by a lox66 sequence and an inverted lox71 sequence (FIG. 21A). In the same lentiviral vector, an EFS promoter drives the expression of Lachnospiraceae bacterium Cpf1 (LbCpf1, or Cpf1 for short) and a puromycin resistance gene (EFS-Cpf1-Puro). After BsmbI restriction digest, the vector linearizes and allows for insertion of a crRNA array. To enable stepwise mutagenesis, crRNA arrays were designed in which the first crRNA is encoded on the sense strand, while the second crRNA is inverted. This construct is referred herein as a crRNA FlipArray. Six consecutive thymidines (6×T) are present in cis at the 3′ end of each crRNA, terminating U6 transcription. Each crRNA is preceded by the LbCpf1 direct repeat (DR) sequence, which guides Cpf1 to process the crRNA array.

Cre-mediated recombination of the lox66 and lox71 mutant loxP sites leads to inversion of the FlipArray, generating a wildtype loxP and a double-mutant loxP, lox72. As the affinity of Cre recombinase for lox72 is substantially lower than for wildtype loxP, inversion of the FlipArray is mostly irreversible. After inversion, the two crRNAs trade places and the second crRNA becomes expressed. Thus, in the absence of Cre, Cpf1 generates indels at the target site of the first crRNA; after Cre recombination, Cpf1 is directed to the target site of the second crRNA. This approach is herein termed Cpf1-Flip. In short, the Cpf1-Flip system leverages CRISPR-Cpf1 mutagenesis and melds it with the inversion capabilities of Cre/lox66/lox71 to enable programmable two-step mutagenesis.

To demonstrate sequential editing of cancer genes, Cpf1-Flip was first applied to generate Neurofibromatosis I (Nf1) and Phosphatase and tensin homolog (Pten) mutations in a mammalian lung cancer cell line (KPD). A FlipArray containing a spacer targeting Nf1 (crNf1) and an inverted spacer targeting Pten (crPten) (crNf1-crPten FlipArray, or NPF) was cloned in. The cells were infected with lentivirus containing EFS-Cpf1-Puro; U6-NPF (FIG. 21B). The pre-recombination construct was designed to only express crRNA targeting the first locus (Nf1) prior to the introduction of Cre. After 6 days of puromycin selection (one week after the initial lentiviral transduction), the cells were then infected with lentivirus containing an EFS promoter driving the expression of Cre (EFS-Cre). Cre-expressing cells undergo inversion of the crRNA FlipArray, leading to sequential mutagenesis at the second locus (Pten) (FIG. 21C).

To detect Cre-mediated inversion of the FlipArray, genomic DNA was isolated from the NPF-expressing lung cancer cells before infection with EFS-Cre and 10 days after infection. Primers were designed that would only generate a product if the FlipArray had successfully inverted (FIG. 22A). Primers specific for the non-inverted FlipArray were also designed. These data demonstrated robust FlipArray inversion (FIG. 22B). Specifically, by D10 following EFS-Cre, the FlipArray inversion frequency was 79.07%±8.23% (mean s.e.m.) (FIG. 22C). In order to monitor the induction of functional FlipArray inversion at the transcript level, total RNA was isolated from the double-infected KPD cells at various timepoints. After cDNA synthesis, inversion-specific primers were utilized to detect inverted crRNA FlipArray transcripts (FIG. 22D). The induction of inverted FlipArray transcripts steadily increased through the course of the experiment, illuminating the kinetics of Cre-mediated inversion of the FlipArray and its subsequent transcription. The low-levels of inverted FlipArray transcripts at baseline could be due to spontaneous inversion, or an artifact of the primer design.

The target sites of crNf1 and crPten were sequenced to determine whether the NPF construct had indeed created mutations in a controlled stepwise manner. Uninfected controls did not have any significant variants at crNf1 or crPten target sites (FIGS. 22E-22K). 7 days following the first lentiviral infection with EFS-Cpf1-Puro; U6-NPF, indels were found at the crNf1 target site, but not the crPten site (FIGS. 22G-22K). Since the second crRNA is not transcribed prior to Cre recombination, this result affirms that inversion of NPF has not yet occurred at this time point. After another 10 days following infection with EFS-Cre lentivirus (17 days following the initial infection with EFS-Cpf1-Puro; U6-NPF), indels were found at both crNf1 and crPten target sites at high frequencies (FIGS. 2I-2K).

To further demonstrate the utility of Cpf1-Flip in diverse biological systems, a FlipArray was designed targeting two human genes, DNA Methyltransferase 1 (DNMT1) and Vascular Endothelial Growth Factor A (VEGFA). The crRNA in the first position targets DNMT1 (crDNMT1) while the second, inverted crRNA targets VEGFA (crVEGFA) (crDNMT1-crVEGFA FlipArray, or DVF) (FIG. 23A). Cre activation induces recombination of the lox66/lox71 sites, such that crVEGFA becomes expressed. Human HEK293T cells were transduced with EFS-Cpf1; U6-DVF lentivirus, followed by puromycin selection. To assess the functionality of the FlipArray, the cells were then infected with EFS-Cre lentivirus. Using primers specific to the non-inverted or inverted DVF FlipArray, it was confirmed that Cre administration drives efficient inversion (FIG. 23B). In this system, inversion efficiency was 85.42%±2.90% by 2 weeks following EFS-Cre (FIG. 23C).

Next, to determine whether the Cpf1-Flip system had enabled sequential mutagenesis at the crDNMT1 and crVEGFA target sites, deep sequencing was performed. As anticipated, uninfected controls did not have significant mutations at either site (FIGS. 23D, 23E, 23J, 23K). Seven days after transduction with EFS-Cpf1; U6-DVF lentivirus, significant indels were found at the crDNMT1 target site but not at the crVEGFA target locus (FIGS. 23F, 23G, 23J, 23K). The cells were then infected with EFS-Cre to cause FlipArray inversion, leading to expression of crVEGFA. Twenty-one days after the initial transduction (14 days after EFS-Cre administration), significant indels were observed at both crDNMT1 and crVEGFA target sites (FIGS. 23H-23I). In these data, the DNMT1 cutting efficiency appeared to be consistently lower at D21 than at D7. This is likely a consequence of random sampling, as only a subset of the D7 cells were subsequently taken forward for Cre infection. In addition, it is possible that DNMTJ loss affects cell viability, given its crucial role in maintaining DNA methylation. The cutting efficiency at crVEGFA was notably lower compared to crDNMT1. This contrast may be due to lower efficiency of the crRNA itself, as well as inefficiencies in FlipArray expression or subsequent crRNA array processing. Taken together, these results demonstrate that Cpf1-Flip is a flexible tool for sequential mutagenesis based on the Cpf1.crRNA complex, temporally controlled by Cre recombinase.

Cpf1-Flip was applied to model acquired resistance to immunotherapy in breast cancer cells (E0771 cell line). A small pool of FlipArrays was designed in which the first crRNA targeted Nf1 while the inverted second crRNA targeted a panel of immunomodulatory factors (Cd274, Ido1, B2m, Fas1, Jak2, and Lgals9; referred to as TSG-Immune FlipArray library). These factors are thought to influence anti-tumor immunity and have been implicated in acquired resistance to checkpoint inhibitors. After pooled lentiviral transduction of E0771 cells with the TSG-Immune FlipArray library, the cells were infected with EFS-Cre lentivirus to induce FlipArray inversion (FIG. 24A). Upon Cre-mediated inversion, the second crRNA is expressed and triggers the knockout of various immunomodulatory factors, thus mimicking the sequential evolution of cancers in the face of immunotherapeutic pressures.

Targeted amplicon sequencing confirmed efficient mutagenesis of Nf1 (FIG. 24B), followed by mutagenesis of the immunomodulatory factors upon Cre-mediated FlipArray inversion (FIG. 24C). Given the pooled nature of these experiments, lower population-level cutting efficiencies are anticipated at the second loci, as only a sixth of the total cell population, on average, is infected with a given FlipArray. The lack of consistent mutagenesis at the crB2m and crCd274 target sites may be intrinsic to the crRNA sequences themselves, a result of inefficient Cre infection/recombination and FlipArray processing, or simply a consequence of biased representation within the cell pool. Of note, high cutting efficiencies at the Jak2 locus were observed despite the pooled nature of the experiment. Since these cells were processed completely in parallel as a minipool, the observation that crJak2 and crLgals9 showed consistent mutagenesis points to intrinsic differences in crRNA targeting efficiencies as the key factor underlying the lack of consistent cutting by crB2m and crCd274. Collectively, these data demonstrate the application of Cpf1-Flip to facilitate sequential genetic screens—for instance, to model the acquisition of resistance mutations to cancer immunotherapy.

The present disclosure provides Cpf1-Flip, an inducible sequential mutagenesis system using invertible crRNA FlipArrays. As a proof-of-concept, sequential mutagenesis were demonstrated in both mouse and human cells, while additionally performing pooled sequential mutagenesis in a cancer cell line. These data revealed that the cutting efficiency of the second target loci can be low with certain crRNAs despite successful FlipArray inversion. The most likely explanation for the discordance between FlipArray inversion and subsequent mutagenesis of the second target locus is the differing efficiencies of the crRNAs themselves. This is corroborated by the variance observed across independent crRNAs in the pooled TSG-Immune library (FIG. 24A-24C), where consistent cutting efficiencies were observed at the Jak2 and Lgals9 target sites, but not at B2m or Cd274. Moreover, cells with different crRNAs in a pool can undergo random drift or selection, further diverting their relative fractions and thereby indel frequencies. Nevertheless, the FlipArray library can be readout by barcoded PCR of the specific crRNA cassette followed by high-throughput sequencing. Thus, as with all CRISPR screens, pooled screen studies using Cpf1-Flip would require multiple independent FlipArrays targeting each gene/gene pair to ensure fair representation in the mutant pool. Optimized crRNA sequences, improved FlipArray designs, and engineered Cpf1 enzymes can improve the consistency and efficiency of Cpf1-Flip.

In certain non-limiting embodiments, by altering the composition and length of the crRNA arrays within the FlipArray, one can readily engineer more complex CRISPR perturbation programs. In other non-limiting embodiments, designs with two or more crRNAs within an invertible FlipArray at baseline can empower stepwise double knockouts (2+2, or quadruple knockouts as an end result) or higher dimensional sequential mutagenesis. In other non-limiting embodiments, the use of modified Cre systems such as CreER, photoactivatable Cre, and split-Cre can provide even greater control of FlipArray inversion. In yet other non-limiting embodiments, utilizing orthogonal recombinases and recognition sites in the crRNA array allows for even more complex multi-step gene editing programs. In yet other non-limiting embodiments, through the use of tethered Cpf1 variants, FlipArrays can also be used for sequential and reversible gene activation, repression, or epigenetic modification (FIG. 25A). Given the scalability and flexibility of FlipArrays, conditional genetic studies for phenotypes that only emerge upon sequential genetic events can be performed using Cpf1-Flip either in culture or in vivo (FIG. 25B). Since new mutations are stochastically acquired by rare individual cells within tumors, Cpf1-Flip can be used for studying the dynamics of rare tumor subclones under varying selection pressures, such as immunotherapy.

In certain non-limiting embodiments, such applications of Cpf1-Flip and its derivatives can be self-contained within a single viral vector, facilitating direct in vivo sequential genetic manipulations and functional studies.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A vector comprising a first long terminal repeat (LTR) sequence, an Embryonal Fyn-Associated Substrate (EFS) sequence, a Cpf1 sequence, a Nuclear Localization Signal (NLS) sequence, an antibiotic resistance sequence, and a second LTR sequence.

2. The vector of claim 1, further comprising a tag sequence.

3. The vector of claim 2, wherein the tag sequence is a Flag2A sequence.

4. The vector of claim 1, wherein the vector comprises the nucleic acid sequence of SEQ ID NO: 1.

5. A vector comprising a first LTR sequence, a promoter sequence, a direct repeat sequence of Cpf1, a first restriction site, a second restriction site, an EFS sequence, an antibiotic resistance sequence, a posttranscriptional regulatory element sequence, and a second LTR sequence.

6. The vector of claim 5, wherein the first and/or second restriction site is a BsmBI restriction site.

7. The vector of claim 5, wherein the posttranscriptional regulatory element sequence comprises a Woodchuck Hepatitis Virus (WHP) Posttranscriptional Regulatory Element (WPRE) sequence.

8. The vector of claim 5, wherein the promoter sequence comprises a U6 promoter sequence.

9. The vector of claim 5, wherein in the vector comprises the nucleic acid sequence of SEQ ID NO: 2.

10. A crRNA array comprising a 5′ nucleotide sequence that is homologous to a first nucleotide sequence on a vector, a first crRNA sequence, a direct repeat sequence of Cpf1, a second crRNA sequence, a terminator sequence, and a 3′ sequence that is homologous to a second sequence on the vector.

11. The crRNA array of claim 10, wherein the vector comprises the vector of claim 5.

12. A vector comprising a first LTR sequence, a promoter sequence, a first direct repeat sequence of Cpf1, a first crRNA sequence, a second direct repeat sequence of Cpf1, a second crRNA sequence, a terminator sequence, an EFS sequence, a posttranscriptional regulatory sequence, and a second LTR sequence.

13. The vector of claim 12, wherein the posttranscriptional regulatory sequence comprises a WPRE sequence.

14. The vector of claim 12, wherein the first crRNA sequence is complementary to a gene selected from the group consisting of Pten and Nf1, and wherein the second crRNA sequence is complementary to a gene selected from the group consisting of Pten and Nf1.

15. A crRNA library comprising a plurality of crRNA arrays cloned into a plurality of vectors, wherein the crRNA arrays individually comprise a 5′ nucleotide sequence that is homologous to a first nucleotide sequence on a vector, a first crRNA sequence, a direct repeat sequence of Cpf1, a second crRNA sequence, a terminator sequence, and a 3′ sequence that is homologous to a second sequence on the vector.

16. The crRNA library of claim 15, wherein the vector comprises the vector of claim 12.

17. The crRNA library of claim 15, wherein the crRNA arrays comprise a nucleotide sequence selected from the group consisting of SEQ ID NOs: 4-9,708.

18. The crRNA library of claim 15, wherein the crRNA arrays comprise a nucleotide sequence selected from the group consisting of SEQ ID NOs: 9,762-21,695.

19. The crRNA library of claim 15, wherein the crRNA library comprises a Cpf1 crRNA array screening (CCAS) library, wherein the crRNA arrays consist of SEQ ID NOs: 4-9,708.

20. The crRNA library of claim 15, wherein the crRNA library comprises a Massively-Parallel crRNA Array Profiling (MCAP) library comprising a plurality of crRNA arrays targeting pairwise combinations of genes significantly mutated in human metastases.

21. The MCAP library of claim 20, wherein the crRNA arrays consist of SEQ ID NOs: 9,762-21,695.

22. A method for simultaneously mutagenizing multiple target sequences in a cell, the method comprising administering to the cell a crRNA library comprising a plurality of vectors comprising a plurality of crRNA arrays, wherein each crRNA array independently comprises a 5′ nucleotide sequence that is homologous to a first nucleotide sequence on the vector, a first crRNA sequence, a direct repeat sequence of Cpf1, a second crRNA sequence, a terminator sequence, and a 3′ sequence that is homologous to a second sequence on the vector, and wherein the first crRNA is complementary to a first target sequence and the second crRNA is complementary to a second target sequence.

23. The method of claim 22, wherein the plurality of crRNA arrays comprise a nucleotide sequence selected from the group consisting of SEQ ID NOs. 4-9,708.

24. The method of claim 22, wherein the plurality of crRNA arrays comprise a nucleotide sequence selected from the group consisting of SEQ ID NOs. 9,762-21,695.

25. The method of claim 22, wherein the terminator sequence comprises a U6 terminator sequence.

26. The method of claim 22, wherein the cell is selected from the group consisting of a T cell, a CD8+ cell, a CD4+ cell, a dendritic cell, an endothelial cell, and a stem cell.

27. The method of claim 22, further comprising wherein the crRNA array comprises at least one additional crRNA sequence that is complementary to at least one additional target sequence.

28. A method of identifying synergistic drivers of transformation and/or tumorigenesis in vivo comprising:
administering a cell mutagenized by a crRNA library to an animal, wherein the crRNA library comprises a plurality of vectors comprising a plurality of crRNA arrays, wherein each crRNA array independently comprises a 5′ nucleotide sequence that is homologous to a first nucleotide sequence on the vector, a first crRNA sequence, a direct repeat sequence of Cpf1, a second crRNA sequence, a terminator sequence and a 3′ sequence that is homologous to a second sequence on the vector, and wherein the first crRNA is complementary to a first target sequence and the second crRNA is complementary to a second target sequence, and
sequencing a nucleotide from a tumor from the animal, and analyzing the data from the sequencing to identify the synergistic drivers of transformation and/or tumorigenesis.

29. An in vivo method for identifying and mapping genetic interactions between a plurality of genes comprising:
administering a cell mutagenized by a crRNA library to an animal, wherein the crRNA library comprises a plurality of vectors comprising a plurality of crRNA arrays, wherein the crRNA array comprises a 5′ nucleotide sequence that is homologous to a first nucleotide sequence on the vector, a first crRNA sequence, a direct repeat sequence of Cpf1, a second crRNA sequence, a terminator sequence, and a 3′ sequence that is homologous to a second sequence on the vector, and wherein the first crRNA is complementary to a first target sequence and the second crRNA is complementary to a second target sequence, and
sequencing a nucleotide from a tissue from the animal, and analyzing the data from the sequencing to identify and map the genetic interactions.

30. The method of claim 28, wherein the plurality of crRNA arrays comprises a nucleotide sequence selected from the group consisting of SEQ ID NOs. 4-9,708.

31. The method of claim 28, wherein the plurality of crRNA arrays comprises a nucleotide sequence selected from the group consisting of SEQ ID NOs. 9,762-21,695.

32. The method of claim 28, further wherein the crRNA array comprises at least one additional crRNA sequence that is complementary to at least one additional target sequence.

33. The method of claim 28, wherein the animal is a mouse.

34. The method of claim 28, wherein the animal is a human.

35. A kit comprising a CCAS library comprising a plurality of vectors comprising a plurality of crRNA arrays, wherein the crRNA arrays comprise a nucleotide sequence selected from the group consisting of SEQ ID NOs: 4-9,708, and instructional material for use thereof.

36. A kit comprising a MCAP library comprising a plurality of vectors comprising a plurality of crRNA arrays, wherein the crRNA arrays comprise a nucleotide sequence selected from the group consisting of SEQ ID NOs: 9,762-21,695, and instructional material for use thereof.

37. The kit of claim 35, wherein the crRNA array comprises at least one additional crRNA sequence that is complementary to at least one additional target sequence.

38. A vector comprising a first promoter, a Cpf1 sequence, a second promoter, a first Cpf1 direct repeat sequence, a lox66 sequence, a second Cpf1 direct repeat sequence, two inverted restriction sites, an inverted lox71 sequence, and a crRNA FlipArray, wherein the crRNA FlipArray comprises a first crRNA sequence, 4-10 consecutive thymidines, a second inverted crRNA sequence, 4-10 consecutive adenines, and a third inverted direct repeat sequence.

39. The vector of claim 38, wherein the vector comprises SEQ ID NO: 21,697.

40. The vector of claim 38, wherein the first promoter is an EFS promoter.

41. The vector of claim 40, wherein the EFS promoter drives expression of Cpf1.

42. The vector of claim 38, wherein the second promoter is a U6 promoter.

43. The vector of claim 42, wherein the U6 promoter drives expression of the crRNA FlipArray.

44. The vector of claim 38, wherein the first promoter and the second promoter are in opposite orientations.

45. The vector of claim 38, further comprising antibiotic resistance marker.

46. The vector of claim 45, wherein the antibiotic resistance marker is a puromycin resistance sequence.

47. The vector of claim 38, wherein the restriction sites are BsmbI restriction sites.

48. The vector of claim 38, wherein the Cpf1 sequence is a Lachnospiraceae bacterium Cpf1 (LbCpf1) sequence.

49. The vector of claim 38, wherein the any one of the first, second, or third, direct repeat sequences is from LbCpf1.

50. A gene editing system capable of inducible, sequential mutagenesis in a cell, the system comprising a vector and a Cre recombinase,
wherein the vector comprising a first promoter, a Cpf1 sequence, a second promoter, a first Cpf1 direct repeat sequence, a lox66 sequence, a second Cpf1 direct repeat sequence, two inverted restriction sites, an inverted lox71 sequence, and a crRNA FlipArray, wherein the crRNA FlipArray comprises a first crRNA sequence, 4-10 consecutive thymidines, a second inverted crRNA sequence, 4-10 consecutive adenines, and a third inverted direct repeat sequence.

51. A gene editing system capable of inducible, sequential mutagenesis in a cell, the system comprising a plurality of vectors and a Cre recombinase,
wherein the vectors comprising a first promoter, a Cpf1 sequence, a second promoter, a first Cpf1 direct repeat sequence, a lox66 sequence, a second Cpf1 direct repeat sequence, two inverted restriction sites, an inverted lox71 sequence, and a crRNA FlipArray, wherein the crRNA FlipArray comprises a first crRNA sequence, 4-10 consecutive thymidines, a second inverted crRNA sequence, 4-10 consecutive adenines, and a third inverted direct repeat sequence.

52. The gene editing system of claim 50, wherein the first crRNA and/or the second crRNA target more than one sequence.

53. A method of inducible, sequential mutagenesis in a cell, the method comprising
administering to the cell a vector comprising a first promoter, a Cpf1 sequence, a second promoter, a first Cpf1 direct repeat sequence, a lox66 sequence, a second Cpf1 direct repeat sequence, two inverted restriction sites, an inverted lox71 sequence, and a crRNA FlipArray, wherein the crRNA FlipArray comprises a first crRNA sequence, 4-10 consecutive thymidines, a second inverted crRNA sequence, 4-10 consecutive adenines, and a third inverted direct repeat sequence,
wherein the first crRNA is expressed,
administering to the cell a Cre recombinase, wherein when the Cre recombinase is administered, the second crRNA is expressed, thus sequentially mutagenizing the cell.

54. The method of claim 53, wherein the cell is a human cell.

55. The method of claim 53, wherein the mutagenesis is selected from the group consisting of nucleotide insertion, nucleotide deletion, frameshift mutation, gene activation, gene repression, and epigenetic modification.

56. The method of claim 53, wherein the first crRNA targets Nf1 and the second crRNA targets Pten.

57. The method of claim 53, wherein the first crRNA and/or the second crRNA targets more than one sequence.

58. A method of inducible, sequential mutagenesis in a cell, the method comprising
administering to the cell a plurality of vectors individually comprising a first promoter, a Cpf1 sequence, a second promoter, a first Cpf1 direct repeat sequence, a lox66 sequence, a second Cpf1 direct repeat sequence, two inverted restriction sites, an inverted lox71 sequence, and a crRNA FlipArray, wherein the crRNA FlipArray comprises a first crRNA sequence, 4-10 consecutive thymidines, a second inverted crRNA sequence, 4-10 consecutive adenines, and a third inverted direct repeat sequence,
wherein the first crRNA is expressed,
administering to the cell a Cre recombinase, wherein when the Cre recombinase is administered, the second crRNA is expressed, thus sequentially mutagenizing the cell.

59. The method of claim 58, wherein the first crRNA and/or the second crRNA targets more than one sequence.

60. The method of claim 58, wherein first crRNA and/or the second crRNA targets a panel of immunomodulatory factors comprising Cd274, Ido1, B2m, Fas1, Jak2, and Lgals9.

61. A method of inducible, sequential mutagenesis in a cell in an animal, the method comprising
administering to the animal a plurality of vectors comprising a first promoter, a Cpf1 sequence, a second promoter, a first Cpf1 direct repeat sequence, a lox66 sequence, a second Cpf1 direct repeat sequence, two inverted restriction sites, an inverted lox71 sequence, and a crRNA FlipArray, wherein the crRNA FlipArray comprises a first crRNA sequence, 4-10 consecutive thymidines, a second inverted crRNA sequence, 4-10 consecutive adenines, and a third inverted direct repeat sequence,
wherein the first crRNA is expressed,
administering to the animal a Cre recombinase, wherein when the Cre recombinase is administered, the second crRNA is expressed thus sequentially mutagenizing the cell in the animal.

62. The vector of claim 38, wherein the first crRNA sequence of the FlipArray comprises six consecutive thymidines.

63. The vector of claim 38, wherein the second crRNA sequence comprises six consecutive adenines.
